(19) United States
(12) Patent Application Publication
(10) Pub. No.: US 2010/0113465 A1
(43) Pub. Date: May 6, 2010

(54) 7-AZASPIRO[3.5]NONANE-7-CARBOXAMIDE COMPOUNDS

(75) Inventors: Scott A. Long, Ballwin, MO (US); Marvin J. Meyers, Wentzville, MO (US); Matthew J. Pelc, Ballwin, MO (US); Barbara A. Schweitzer, St. Louis, MO (US); Atli Thorarensen, Dardenne Prairie, MO (US); Jane L. Wang, Wildwood, MO (US)

Correspondence Address: PFIZER INC;Mary J Hosley, 150 EAST 42ND STREET, MS: 150/02/E112, NEW YORK, NY, 10017-5612, US

(73) Assignee: Pfizer Inc.

(21) Appl. No.: 12/573,897

(22) Filed: Oct. 6, 2009

Related U.S. Application Data
(60) Provisional application No. 61/121,349, filed on Dec. 10, 2008.
(60) Provisional application No. 61/109,712, filed on Oct. 30, 2008.

(51) Int. Cl. A61K 31/501; C07D 221/20; A61K 31/438; A61P 29/00; C07D 401/14

(52) U.S. Cl. 514/252.03; 546/16; 514/278; 544/238

(57) ABSTRACT

Provided herein are 7-azaspiro[3.5]nonane-7-carboxamide compounds and the pharmaceutically acceptable salts of such compounds useful in treating diseases or conditions associated with fatty acid amide hydrolase (FAAH) activity, conditions including including acute pain, chronic pain, neuropathic pain, nociceptive pain, inflammatory pain, cancer and cancer pain, fibromyalgia, rheumatoid arthritis, inflammatory bowel disease, lupus, diabetes, allergic asthma, vascular inflammation, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, or cardiovascular disease.

### FIELD OF THE INVENTION

The present invention relates to 7-azaspiro[3.5]nonane-7-carboxamide compounds and the pharmaceutically acceptable salts of such compounds. The invention also relates to the processes for the preparation of the compounds, intermediates used in their preparation, compositions containing the compounds, and the uses of the compounds in treating diseases or conditions associated with fatty acid amide hydrolase (FAAH) activity.

### BACKGROUND OF THE INVENTION

Fatty acid amides represent a family of bioactive lipids with diverse cellular and physiological effects. Fatty acid amides are hydrolyzed to their corresponding fatty acids by an enzyme known as fatty acid amide hydrolase (FAAH). FAAH is a mammalian integral membrane serine hydrolase responsible for the hydrolysis of a number of primary and secondary fatty acid amides, including the neuromodulatory compounds anandamide and oleamide. Anandamide (arachidonoyl ethanolamide) has been shown to possess cannabinoid-like analgesic properties and is released by stimulated neurons. The effects and endogenous levels of anandamide increase with pain stimulation, implying its role in suppressing pain neurotransmission and behavioral analgesia. Supporting this, FAAH inhibitors that elevate brain anandamide levels have demonstrated efficacy in animal models of pain, inflammation, anxiety, and depression. Lichtman, A. H. et al. (2004), J. Pharmacol. Exp. Ther. 311, 441-448; Jayamanne, A. et al. (2006), Br. J. Pharmacol. 147, 281-288; Kathuria, S. et al. (2003), Nature Med., 9, 76-81; Piomelli D. et al. (2005), Proc. Natl. Acad. Sci. 102, 18620-18625.

Further recent reviews on this subject are as follows:

Ahn, Kay; McKinney, Michele K.; Cravatt, Benjamin F, Chemical Reviews (Washington, D.C., United States) (2008), 108(5), 1687-1707;

Ahn, Kay; Johnson, Douglas S.; Cravatt, Benjamin F, Expert Opin. Drug Discov. (2009) 4(7), pp 763-784;

M Seierstad and J. G. Breitenbucher, Discovery and Development of Fatty Acid Amide Hydrolase (FAAH) Inhibitors, J. Med. Chem. XXXX, vol. xxx, no. xx, Published on Web Nov. 5, 2008.

WO 2006/085196 teaches a method for measuring activity of an ammonia-generating enzyme, such as FAAH. WO 2006/067613 teaches compositions and methods for expression and purification of FAAH. WO 2008/047229 teaches biaryl ether urea compounds useful for treating FAAH-mediated conditions. WO2006/074025 concerns piperazinyl and piperidinyl ureas as FAAH modulators.

There remains a need for new compounds that are inhibitors of FAAH and, therefore, are useful in the treatment of a wide range of disorders, including pain.

### SUMMARY OF THE INVENTION

Provided herein are compounds of the Formula I:

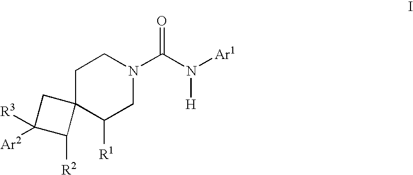

wherein:

Ar¹ is selected from:

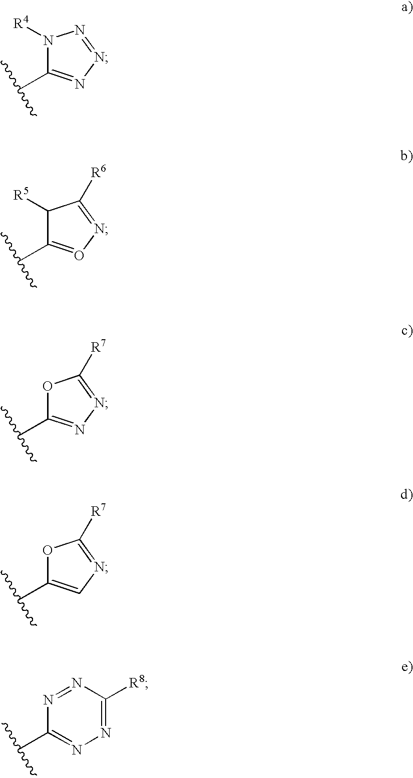

f) benzoisoxazole optionally substituted by 1 to 3 substituents selected from halo, C₁-C₃ alkyl, C₁-C₃ alkoxy, C₁-C₃ haloalkyl or C₁-C₃ haloalkoxy; or
g) pyridine, pyridazine, pyrimidine, or pyrazine; wherein the pyridine, pyridazine, pyrimidine, or pyrazine is optionally substituted by 1 to 3 halo, C₁-C₃ alkyl, —(CH₂)ₙ—(C₃-C₆ cycloalkyl), C₁-C₃ alkoxy, C₁-C₃ haloalkyl or C₁-C₃ haloalkoxy substituents;

Ar² is selected from:
a) phenyl optionally substituted by 1 to 5 substituents selected from halo, C₁-C₆ alkyl, —(CH₂)ₙ—(C₃-C₆ cycloalkyl), C₁-C₆ alkoxy, —(CH₂)ₙ—(C₃-C₆ cycloalkoxy), C₁-C₆ haloalkyl, C₁-C₆ haloalkoxy, —O—CH₂—CH₂—O—(C₁-C₆ alkyl), or —O—CH₂—CH₂—O—(C₁-C₆ haloalkyl); wherein the phenyl is optionally substituted by a substituent of the formulae —R⁹, —O—R⁹, —O—(CH₂)ₚ—R⁹, or —(CH₂)ₚ—O—R⁹;
b) oxazole, isoxazole, thiazole, isothiazole, oxadiazole, or thiadiazole substituted by a substituent of the formulae —(CH₂)ₙ—R⁹, —(CH₂)ₘ—O—R⁹, or —(CH₂)ₚ—O—(CH₂)ₚ—R⁹;
c) a heterocycle of the formula:

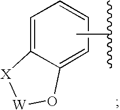

wherein X is CH₂ or O, and W is (CH₂)ₘ or CF₂; or
d) naphthyl, quinolinyl or isoquinolinyl optionally substituted by 1 to 3 halo, C₁-C₃ alkyl, C₁-C₃ alkoxy, C₁-C₃ haloalkyl or C₁-C₃ haloalkoxy substituents;

wherein if Ar¹ is pyridine, pyridazine, pyrimidine, or pyrazine, then Ar² must be phenyl substituted by —O—R⁹;

R¹ and R² are independently selected from hydrogen, F, or CH₃;

R³ is hydrogen, CH₃, —O—CH₃, OH, CN, or F;

R⁴ is hydrogen, C₁-C₆ alkyl, —(CH₂)ₙ—(C₃-C₆ cycloalkyl), or C₁-C₆ haloalkyl;

R⁵ is C₁-C₃ alkyl;

R⁶ is hydrogen, C₁-C₆ alkyl, or C₁-C₃ haloalkyl;

R⁷ is C₁-C₃ alkyl, —(CH₂)ₙ—(C₃-C₆ cycloalkyl), R⁹, or —CH₂—O—R⁹;

R⁸ is phenyl optionally substituted by from 1 to 3 substituents selected from halo, C₁-C₃ alkyl, C₁-C₃ alkoxy, C₁-C₃ haloalkyl or C₁-C₃ haloalkoxy groups;

R⁹ is selected from phenyl, naphthyl, or heteroaryl; wherein R⁹ is optionally substituted by from 1 to 3 substituents selected from halo, C₁-C₃ alkyl, —(CH₂)ₙ—(C₃-C₆ cycloalkyl), C₁-C₃ alkoxy, —(CH₂)ₙ—(C₃-C₆ cycloalkoxy), C₁-C₃ haloalkyl, or C₁-C₃ haloalkoxy;

m is 1, 2 or 3; n is 0, 1, 2, 3 or 4; and p is 1 or 2;

or a pharmaceutically acceptable salt thereof.

Also provided are pharmaceutical compositions comprising a therapeutically effective amount of a compound herein, or a pharmaceutically acceptable salt thereof, and a pharmaceutically acceptable carrier. Further provided herein are methods of treating FAAH-mediated diseases or conditions.

### DETAILED DESCRIPTION

Provided herein are compounds of Formula I:

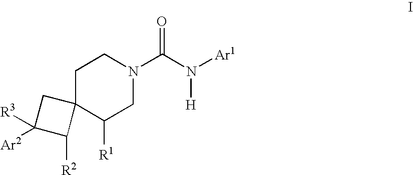

wherein:

Ar¹ is selected from:

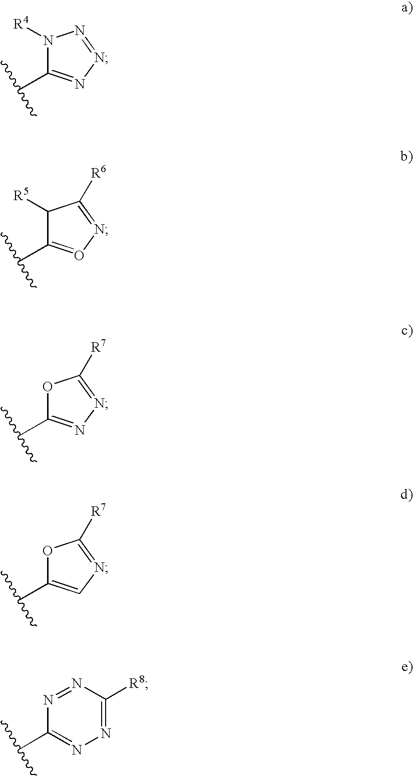

f) benzoisoxazole optionally substituted by 1 to 3 substituents selected from halo, C₁-C₃ alkyl, C₁-C₃ alkoxy, C₁-C₃ haloalkyl or C₁-C₃ haloalkoxy; or
g) pyridine, pyridazine, pyrimidine, or pyrazine; wherein the pyridine, pyridazine, pyrimidine, or pyrazine is optionally substituted by 1 to 3 halo, C₁-C₃ alkyl, —(CH₂)ₙ—(C₃-C₆ cycloalkyl), C₁-C₃ alkoxy, C₁-C₃ haloalkyl or C₁-C₃ haloalkoxy substituents;

Ar² is selected from:
a) phenyl optionally substituted by 1 to 5 substituents selected from halo, C₁-C₆ alkyl, —(CH₂)ₙ—(C₃-C₆ cycloalkyl), C₁-C₆ alkoxy, —(CH₂)ₙ—(C₃-C₆ cycloalkoxy), C₁-C₆ haloalkyl, C₁-C₆ haloalkoxy, —O—CH₂—CH₂—O—(C₁-C₆ alkyl), or —O—CH₂—CH₂—O—(C₁-C₆ haloalkyl); wherein the phenyl is optionally substituted by a substituent of the formulae —R⁹, —O—R⁹, —O—(CH₂)ₚ—R⁹, or —(CH₂)ₚ—O—R⁹;
b) oxazole, isoxazole, thiazole, isothiazole, oxadiazole, or thiadiazole substituted by a substituent of the formulae —(CH₂)ₙ—R⁹, —(CH₂)ₘ—O—R⁹, or —(CH₂)ₚ—O—(CH₂)ₚ—R⁹;
c) a heterocycle of the formula:

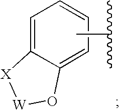

wherein X is CH₂ or O, and W is (CH₂)ₘ or CF₂; or
d) naphthyl, quinolinyl or isoquinolinyl optionally substituted by 1 to 3 halo, C₁-C₃ alkyl, C₁-C₃ alkoxy, C₁-C₃ haloalkyl or C₁-C₃ haloalkoxy substituents;

wherein if Ar¹ is pyridine, pyridazine, pyrimidine, or pyrazine, then Ar² must be phenyl substituted by —O—R⁹;

R¹ and R² are independently selected from hydrogen, F, or CH₃; R³ is hydrogen, CH₃, —O—CH₃, OH, CN, or F; R⁴ is hydrogen, C₁-C₆ alkyl, —(CH₂)ₙ—(C₃-C₆ cycloalkyl), or C₁-C₆ haloalkyl; R⁵ is C₁-C₃ alkyl; R⁶ is hydrogen, C₁-C₆ alkyl, or C₁-C₃ haloalkyl; R⁷ is C₁-C₃ alkyl, —(CH₂)ₙ—(C₃-C₆ cycloalkyl), R⁹, or —CH₂—O—R⁹; R⁸ is phenyl optionally substituted by from 1 to 3 substituents selected from halo, C₁-C₃ alkyl, C₁-C₃ alkoxy, C₁-C₃ haloalkyl or C₁-C₃ haloalkoxy;

R⁹ is selected from phenyl, naphthyl, or heteroaryl; wherein R⁹ is optionally substituted by from 1 to 3 substituents selected from halo, C₁-C₃ alkyl, —(CH₂)ₙ—(C₃-C₆ cycloalkyl), C₁-C₃ alkoxy, —(CH₂)ₙ—(C₃-C₆ cycloalkoxy), C₁-C₃ haloalkyl or C₁-C₃ haloalkoxy; m is 1, 2 or 3; n is 0, 1, 2, 3 or 4; and p is 1 or 2; or a pharmaceutically acceptable salt thereof.

Further provided are compounds within the groups of compounds described above wherein Ar² is selected from:
a) phenyl optionally substituted by from 1 to 3 substituents selected from F, Cl, methyl, ethyl, CF₃, OCH₃, or OCF₃; wherein the phenyl may also be substituted by a substituent of the formulae —O—R⁹ or —O—CH₂—CH₂—O—R⁹;
b) thiazole or oxadiazole substituted by a substituent of the formulae —R⁹; or
c) 2,2,-difluoro-1,3-benzodioxole;

R¹ and R² are hydrogen;

R⁴, R⁵, and R⁶ are methyl;

wherein if Ar² is phenyl, R⁹ is pyridine or pyrimidine, the pyridine or pyrimidine being optionally substituted by from 1 to 3 substituents selected from F, Cl, Br, CF₃, or OCF₃; and if Ar² is thiazole or oxadiazole, R⁹ is phenyl optionally substituted by from 1 to 3 substituents selected from F, Cl, Br, CF₃, or OCF₃; or a pharmaceutically acceptable salt thereof.

Within each of the groups of compounds, and salts thereof, described herein are subgroups in which the variables R¹, R² and R³ are each hydrogen. It is understood that the optional substituents on the Ar¹ and Ar² groups described herein are selected independently and each ring so described may contain the number of listed substituents that are the same or different from each other.

Also provided within each of the groups of compounds described herein is a subset of compounds, including pharmaceutically acceptable salts thereof, wherein R⁹, when present, is phenyl, pyridine or pyrimidine, each optionally by from 1 to 3 substituents selected from halo, C₁-C₃ alkyl, —(CH₂)ₙ—(C₃-C₆ cycloalkyl), C₁-C₃ alkoxy, —(CH₂)ₙ—(C₃-C₆ cycloalkoxy), C₁-C₃ haloalkyl or C₁-C₃ haloalkoxy; and n is 0, 1, 2, 3 or 4. Within each of these groups is a further subset wherein R⁹ is optionally substituted by 1 to 3 substituents selected from F, Cl, Br, CF₃, or OCF₃; or a pharmaceutically acceptable salt thereof.

Further provided within each of the groups of compounds described herein are compounds wherein:

Ar¹ is selected from:

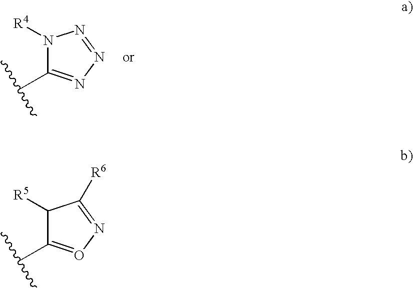

Ar² is selected from formulae, wherein R, R′, and Z are as defined under each formula:

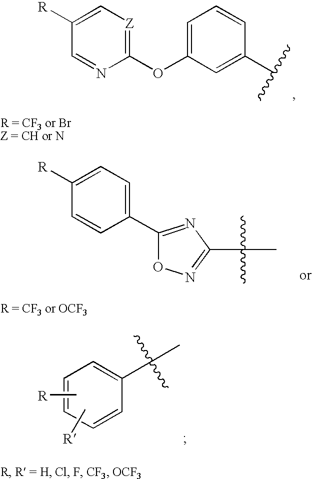

R¹ and R² are H; R³ is H or F; and R⁴, R⁵, and R⁶ are methyl; or a pharmaceutically acceptable salt thereof.

Provided are compounds within each of the groups described herein in which Ar² is:

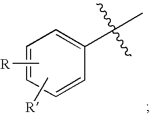

wherein R is F, Cl, CF₃ or OCF₃; and R′ is H or F; or a pharmaceutically acceptable salt thereof.

Also further provided within the groups of compounds described are compounds wherein Ar² is:

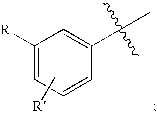

wherein R is F, Cl, CF₃ or OCF₃; and R′ is H or F; or a pharmaceutically acceptable salt thereof.

Also provided within each of the groups of compounds described herein are compounds wherein, when Ar² is oxadiazole, the oxadiazole is 1,2,4-oxadiazole; or a pharmaceutically acceptable salt thereof. Also provided within each of the groups of compounds described herein are compounds wherein, when Ar² is thiazole, the thiazole is 1,3-thiazole; or a pharmaceutically acceptable salt thereof.

In each of the groups described herein it is understood that, when a list of optional substituents is provided, each of the substituents is independently selected from the group of substituents.

Preferable groups of compounds of formula I and their pharmaceutically acceptable salts are those wherein independently:

R¹ has the value of R¹ of any of the specific compounds mentioned below;

R² has the value of R² of any of the specific compounds mentioned below;

R³ has the value of R³ of any of the specific compounds mentioned below;

Ar¹ has the value of Ar¹ of any of the specific compounds mentioned below; and

Ar² has the value of Ar² of any of the specific compounds mentioned below.

The most preferable compounds of formula I and their pharmaceutically acceptable salts are the compounds specifically mentioned below and their pharmaceutically acceptable salts.

Also provided are pharmaceutical compositions comprising a therapeutically effective amount of a compound herein, or a pharmaceutically acceptable salt thereof, and a pharmaceutically acceptable carrier. Further provided herein are methods of treating FAAH-mediated diseases or conditions including acute pain, chronic pain, neuropathic pain, nociceptive pain, inflammatory pain, cancer and cancer pain, fibromyalgia, rheumatoid arthritis, inflammatory bowel disease, lupus, diabetes, allergic asthma, vascular inflammation, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, or cardiovascular disease in a subject by administering to a subject in need thereof a therapeutically effective amount of one or more of the compounds herein, or a pharmaceutically acceptable salt thereof. Provided herein is also the use of a compound described herein, or a pharmaceutically acceptable salt thereof, in the manufacture of a medicament for the treatment of a FAAH-mediated disease or condition. Individual methods using a compound described herein, or a pharmaceutically acceptable salt thereof, in the manufacture of a medicament for the treatment of each of the individual diseases or conditions described herein are also provided.

This disclosure uses the definitions provided below. Some chemical formulae may include a dash (“—”) to indicate a bond between atoms or indicate a point of attachment. “Substituted” groups are those in which one or more hydrogen atoms have been replaced with one or more non-hydrogen atoms or groups, the “substituents”. “Alkyl” refers to straight chain or branched chain saturated hydrocarbon groups, generally having a specified number of carbon atoms (i.e., C₁-C₆alkyl). “Alkoxy” refers to alkyl-O— groups wherein the alkyl portions may be straight chain or branched, such as methoxy, ethoxy, n-propoxy, and i-propoxy groups. “Halo,” or “halogen” may be used interchangeably, and are fluoro, chloro, bromo, and iodo. The terms “haloalkyl”, “haloalkoxy” or “—O-haloalkyl” refer, respectively, to alkyl or alkoxy groups substituted by one or more halogens. Examples include —CF₃, —CH₂—CF₃, —CF₂—CF₃, —O—CF₃, and —OCH₂—CF₃. “Cycloalkyl” refers to saturated monocyclic and bicyclic hydrocarbon rings, generally having a specified number of carbon atoms that comprise the ring (i.e. C₃-C₆ cycloalkyl), optionally including one or more substituents. Examples of monocyclic cycloalkyl groups include cyclopropyl, cyclobutyl, cyclopentyl, cyclohexyl, and the like. “Cycloalkoxy” or “—O-cycloalkyl” refer to cycloalkyl groups attached through an oxygen atom, such as cyclopropoxy, cyclobutoxy, cyclopentoxy, and cyclohexoxy groups. The abbreviations R.T., RT, r.t. or rt refer to “room temperature”.

“Heteroaryl” and “heteroarylene” refer to monovalent or divalent aromatic groups, respectively, containing from 1 to 4 ring heteroatoms selected from O, S or N. Examples of monocyclic heteroaryl groups include pyrrolyl, furanyl, thiophenyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, and the like.

Heteroaryl and heteroarylene groups also include bicyclic groups, including fused ring systems wherein at least one ring is aromatic. Examples of bicyclic heteroaryl groups include benzofuranyl, benzothiopheneyl, indolyl, benzoxazolyl, benzodioxazolyl, benzimidazolyl, indazolyl, benzotriazolyl, benzothiofuranyl, benzothiazolyl, benzotriazolyl, benzotetrazolyl, benzoisoxazolyl, benzoisothiazolyl, benzoimidazolinyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, purinyl, indolizinyl, imidazo[1,2-a]pyridinyl, imidazo[1,5-a]pyridinyl, pyrazolo[1,5-a]pyridinyl, pyrrolo[1,2-b]pyridinyl, and imidazo[1,2-c]pyridinyl. Other examples include quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, pyrimido[4,5-d]pyrimidinyl, isobenzofuranyl, isochromanyl, pteridinyl, oxazolo[5,4-c]pyridinyl, oxazolo[4,5-c]pyridinyl, oxazolo[5,4-b]pyridinyl, oxazolo[4,5-b]pyridinyl, isoxazolopyridinyl, thiazolylpyridinyl, oxazolopyrimidinyl, and the like.

“Subject” refers to a mammal, including humans, as well as companion animals, such as dogs and cats, and commercial or farm mammals, such as hogs, cattle, horses, goats, sheep, rabbits, etc. “Treating” refers to reversing, alleviating, inhibiting the progress of a disorder or condition to which such term applies, or to reversing, alleviating, inhibiting the progress of, or preventing one or more symptoms of such disorder or condition. “Therapeutically effective amount” refers to the quantity of a compound that may be used for treating a subject, which amount may depend on the subject's weight and age and the route of administration, among other things. “Excipient” or “adjuvant” refers to any substance in a pharmaceutical formulation that is not an active pharmaceutical ingredient (API). “Pharmaceutical composition” refers to a combination of one or more drug substances and one or more excipients. “Drug product,” “pharmaceutical dosage form,” “dosage form,” “final dosage form” and the like, refer to a pharmaceutical composition that is administered to a subject in need of treatment and generally may be in the form of tablets, capsules, liquid solutions, suspensions, patches, films, and the like.

Pharmaceutically acceptable carriers are understood to be agents, other than the active pharmacological ingredients, used in the preparation, maintenance or delivery of pharmaceutical formulations. Non-limiting examples of classes of pharmaceutically acceptable carriers include fillers, binders, disintegrants, bulking agents, lubricants, colorants, solubilizing agents, adjuvants, excipients, coating agents, glidants, diluents, emulsifiers, solvents, surfactants, emollients, adhesives, anti-adherents, wetting agents, sweeteners, flavoring agents, antioxidants, alkalizing agents, acidifiers, buffers, adsorbents, stabilizing agents, suspending agents, preservatives, plasticizers, nutrients, bioadhesives, extended and controlled release agents, stiffening agents, humectants, penetration enhancers, chelating agents, and the like.

The compounds herein and the pharmaceutically acceptable salts thereof, which includes those of Formula I, may be used to treat acute pain, chronic pain, neuropathic pain, nociceptive pain, inflammatory pain, fibromyalgia, rheumatoid arthritis, inflammatory bowel disease, lupus, diabetes, allergic asthma, vascular inflammation, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, and cardiovascular disease.

Physiological pain is a protective mechanism designed to warn of danger from potentially injurious stimuli from the external environment and may be classified as acute or chronic. Acute pain begins suddenly, is short-lived (usually 12 weeks or less), is usually associated with a specific cause, such as a specific injury, and is often sharp and severe. Acute pain does not generally result in persistent psychological response. Chronic pain is long-term pain, typically lasting for more than 3 months and leading to psychological and emotional problems. Examples of chronic pain are neuropathic pain (e.g. painful diabetic neuropathy, postherpetic neuralgia), carpal tunnel syndrome and back, headache, cancer, arthritic and chronic post-surgical pain.

Clinical pain is present when discomfort and abnormal sensitivity feature among the patient's symptoms, including 1) spontaneous pain which may be dull, burning, or stabbing; 2) exaggerated pain responses to noxious stimuli (hyperalgesia); and 3) pain produced by normally innocuous stimuli (allodynia). Although patients suffering from various forms of acute and chronic pain may have similar symptoms, the underlying mechanisms may be different and require different treatment strategies. Pain can also be divided into different subtypes according to differing pathophysiology, including nociceptive, inflammatory and neuropathic pain. Nociceptive pain is induced by tissue injury or by intense stimuli with the potential to cause injury. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumor related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is defined as pain initiated or caused by a primary lesion or dysfunction in the nervous system. Nerve damage can be caused by trauma and disease and the term ‘neuropathic pain’ encompasses many disorders with diverse etiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life. The symptoms of neuropathic pain include spontaneous pain, which can be continuous, and paroxysmal or abnormal evoked pain, such as hyperalgesia (increased sensitivity to a noxious stimulus) and allodynia (sensitivity to a normally innocuous stimulus).

Another type of inflammatory pain is visceral pain which includes pain associated with inflammatory bowel disease (IBD). Visceral pain is pain associated with the viscera, which encompass the organs of the abdominal cavity, including the sex organs, spleen and part of the digestive system. Visceral pain can be divided into digestive visceral pain and non-digestive visceral pain. Commonly encountered gastrointestinal, (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Visceral pain includes that associated with dysmenorrhea, cystitis and pancreatitis and pelvic pain.

Some types of pain have multiple etiologies and thus can be classified in more than one area, e.g. back pain and cancer pain have both nociceptive and neuropathic components. Other types of pain include pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis; heart and vascular pain, including pain caused by angina, myocardical infarction, mitral stenosis, pericarditis, Raynaud's phenomenon, scleredoma and skeletal muscle ischemia; head pain, such as migraine (including migraine with aura and migraine without aura), cluster headache, tension-type headache mixed headache and headache associated with vascular disorders; and orofacial pain, including dental pain, otic pain, burning mouth syndrome and temporomandibular myofascial pain.

As described above, the compounds herein, and the pharmaceutically acceptable salts thereof, may be used to treat CNS disorders, including schizophrenia and other psychotic disorders, mood disorders, anxiety disorders, sleep disorders, and cognitive disorders, such as delirium, dementia, and amnestic disorders. The standards for diagnosis of these disorders may be found in the American Psychiatric Association's Diagnostic and Statistical Manual of Mental Disorders (4th ed., 2000), which is commonly referred to as the DSM Manual.

For the purposes of this disclosure, schizophrenia and other psychotic disorders include schizophreniform disorder, schizoaffective disorder, delusional disorder, brief psychotic disorder, shared psychotic disorder, psychotic disorder due to general medical condition, and substance-induced psychotic disorder, as well as medication-induced movement disorders, such as neuroleptic-induced Parkinsonism, neuroleptic malignant syndrome, neuroleptic-induced acute dystonia, neuroleptic-induced acute akathisia, neuroleptic-induced tardive dyskinesia, and medication-induced postural tremor. Mood disorders include depressive disorders, such as major depressive disorder, dysthymic disorder, premenstrual dysphoric disorder, minor depressive disorder, recurrent brief depressive disorder, postpsychotic depressive disorder of schizophrenia, and major depressive episode with schizophrenia; bipolar disorders, such as bipolar I disorder, bipolar II disorder, cyclothymia, and bipolar disorder with schizophrenia; mood disorders due to general medical condition; and substance-induced mood disorders. Anxiety disorders include panic attack, agoraphobia, panic disorder without agoraphobia, agoraphobia without history of panic disorder, specific phobia, social phobia (social anxiety disorder), obsessive-compulsive disorder, posttraumatic stress disorder, acute stress disorder, generalized anxiety disorder, anxiety disorder due to general medical condition, substance-induced anxiety disorder, and mixed anxiety-depressive disorder. Sleep disorders include primary sleep disorders, such as dyssomnias (primary insomnia, primary hypersomnia, narcolepsy, breathing-related sleep disorder, circadian rhythm sleep disorder, sleep deprivation, restless legs syndrome, and periodic limb movements) and parasomnias (nightmare disorder, sleep terror disorder, sleepwalking disorder, rapid eye movement sleep behavior disorder, and sleep paralysis); sleep disorders related to another mental disorder, including insomnia related to schizophrenia, depressive disorders, or anxiety disorders, or hypersomnia associated with bipolar disorders; sleep disorders due to a general medical condition; and substance-induced sleep disorders, Delirium, dementia, and amnestic and other cognitive disorders, includes delirium due to a general medical condition, substance-induced delirium, and delirium due to multiple etiologies; dementia of the Alzheimer's type, vascular dementia, dementia due to general medical conditions, dementia due to human immunodeficiency virus disease, dementia due to head trauma, dementia due to Parkinson's disease, dementia due to Huntington's disease, dementia due to Pick's disease, dementia due to Creutzfeldt-Jakob disease, dementia due to other general medical conditions, substance-induced persisting dementia, dementia due to multiple etiologies; amnestic disorders due to a general medical condition, and substance-induced persisting amnestic disorder.

Substance-induced disorders refer to those resulting from the using, abusing, dependence on, or withdrawal from, one or more drugs or toxins, including alcohol, amphetamines or similarly acting sympathomimetics, caffeine, cannabis, cocaine, hallucinogens, inhalants, nicotine, opioids, phencyclidine or similarly acting arylcyclohexylamines, and sedatives, hypnotics, or anxiolytics, among others.

Urinary incontinence includes the involuntary or accidental loss of urine due to the inability to restrain or control urinary voiding. Urinary incontinence includes mixed urinary incontinence, nocturnal enuresis, overflow incontinence, stress incontinence, transient urinary incontinence, and urge incontinence.

The compounds described and specifically named herein may form pharmaceutically acceptable complexes, salts, solvates and hydrates. The salts include acid addition salts (including di-acids) and base salts.

Pharmaceutically acceptable acid addition salts include salts derived from inorganic acids such as hydrochloric acid, nitric acid, phosphoric acid, sulfuric acid, hydrobromic acid, hydroiodic acid, hydrofluoric acid, and phosphorous acids, as well salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride, chloride, hydrobromide, bromide, hydroiodide, iodide, isethionate, lactate, malate, maleate, malonate, mesylate, methylsulfate, naphthylate, 2-napsylate, nicotinate, nitrate, orotate, oxalate, almitate, pamoate, phosphate, hydrogen phosphate, dihydrogen phosphate, pyroglutamate, saccharate, stearate, succinate, tannate, tartrate, tosylate, trifluoroacetate and xinofoate salts.

Pharmaceutically acceptable base salts include salts derived from bases, including metal cations, such as an alkali or alkaline earth metal cation, as well as amines. Examples of suitable metal cations include sodium (Na⁺), potassium (K⁺), magnesium (Mg²⁺), calcium (Ca²⁺), zinc (Zn²⁺), and aluminum (Al³⁺). Examples of suitable amines include arginine, N,N′-dibenzylethylenediamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine.

Pharmaceutically acceptable salts may be prepared using various methods. For example, one may react a compound with an appropriate acid or base to give the desired salt. One may also react a precursor of the compound with an acid or base to remove an add- or base-labile protecting group or to open a lactone or lactam group of the precursor. Additionally, one may convert a salt of the compound to another salt through treatment with an appropriate acid or base or through contact with an ion exchange resin. Following reaction, one may then isolate the salt by filtration if it precipitates from solution, or by evaporation to recover the salt. The degree of ionization of the salt may vary from completely ionized to almost non-ionized.

The compounds herein, and the pharmaceutically acceptable salts thereof, may exist in a continuum of solid states ranging from fully amorphous to fully crystalline. They may also exist in unsolvated and solvated forms. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, d₆-acetone, d₆-DMSO).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will be the norm.

The compounds herein, and the pharmaceutically acceptable salts thereof, may also exist as multi-component complexes (other than salts and solvates) in which the compound and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together.

“Prodrugs” refer to compounds that when metabolized in vivo, undergo conversion to compounds having the desired pharmacological activity. Prodrugs may be prepared by replacing appropriate functionalities present in pharmacologically active compounds with “pro-moieties” as described, for example, in H. Bundgaar, Design of Prodrugs (1985). Examples of prodrugs include ester, ether or amide derivatives of the compounds herein, and their pharmaceutically acceptable salts.

“Metabolites” refer to compounds formed in vivo upon administration of pharmacologically active compounds. Examples include hydroxymethyl, hydroxy, secondary amino, primary amino, phenol, and carboxylic acid derivatives of compounds herein, and the pharmaceutically acceptable salts thereof having methyl, alkoxy, tertiary amino, secondary amino, phenyl, and amide groups, respectively. Geometrical (cis/trans) isomers may be separated by conventional techniques such as chromatography and fractional crystallization. “Tautomers” refer to structural isomers that are interconvertible via a low energy barrier. Tautomeric isomerism (tautomerism) may take the form of proton tautomerism in which the compound contains, for example, an imino, keto, or oxime group, or valence tautomerism in which the compound contains an aromatic moiety.

The compounds herein, and pharmaceutically acceptable salts thereof, can be administered as crystalline or amorphous forms, prodrugs, metabolites, hydrates, solvates, complexes, and tautomers thereof, as well as all isotopically-labelled compounds thereof. They may be administered alone or in combination with one another or with one or more other pharmacologically active compounds. Generally, one or more these compounds are administered as a pharmaceutical composition (a formulation) in association with one or more pharmaceutically acceptable excipients.

Also provided herein are pharmaceutical compositions comprising a therapeutically effective amount of a compound described herein, or a pharmaceutically acceptable salt thereof, and on or more pharmaceutically acceptable carriers and/or excipients. The compounds herein, and the pharmaceutically acceptable salts thereof, may be administered orally. Oral administration may involve swallowing in which case the compound enters the bloodstream via the gastrointestinal tract. Alternatively or additionally, oral administration may involve mucosal administration (e.g., buccal, sublingual, supralingual administration) such that the compound enters the bloodstream through the oral mucosa. Formulations suitable for oral administration include solid, semi-solid and liquid systems such as tablets; soft or hard capsules containing multi- or nano-particulates, liquids, or powders; lozenges which may be liquid-filled; chews; gels; fast dispersing dosage forms; films; ovules; sprays; and buccal or mucoadhesive patches. Liquid formulations include suspensions, solutions, syrups and elixirs. Such formulations may be employed as fillers in soft or hard capsules (made, for example, from gelatin or hydroxypropyl methylcellulose) and typically comprise a carrier (e.g., water, ethanol, polyethylene glycol, propylene glycol, methylcellulose, or a suitable oil) and one or more emulsifying agents, suspending agents or both. Liquid formulations may also be prepared by the reconstitution of a solid (e.g., from a sachet).

The compounds herein, and the pharmaceutically acceptable salts thereof, may also be used in fast-dissolving, fast-disintegrating dosage forms such as those described in Liang and Chen, Expert Opinion in Therapeutic Patents, 11(6):981-986 (2001).

For tablet dosage forms, depending on dose, the active pharmaceutical ingredient (API) may comprise from about 1 wt % to about 80 wt % of the dosage form or more typically from about 5 wt % to about 60 wt % of the dosage form. In addition to the API, tablets may include one or more disintegrants, binders, diluents, surfactants, glidants, lubricants, anti-oxidants, colorants, flavoring agents, preservatives, and taste-masking agents. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, C₁₋₆ alkyl-substituted hydroxypropylcellulose, starch, pregelatinized starch, and sodium alginate. Generally, the disintegrant will comprise from about 1 wt % to about 25 wt % or from about 5 wt % to about 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropylcellulose and hydroxypropylmethylcellulose. Tablets may also contain diluents, such as lactose (monohydrate, spray-dried monohydrate, anhydrous), mannitol, xylitol, dextrose, sucrose, sorbitol, microcrystalline cellulose, starch and dibasic calcium phosphate dihydrate. Tablets may also include surface active agents, such as sodium lauryl sulfate and polysorbate 80, and glidants such as silicon dioxide and talc. When present, surface active agents may comprise from about 0.2 wt % to about 5 wt % of the tablet, and glidants may comprise from about 0.2 wt % to about 1 wt % of the tablet. Tablets may also contain lubricants such as magnesium stearate, calcium stearate, zinc stearate, sodium stearyl fumarate, and mixtures of magnesium stearate with sodium lauryl sulfate. Lubricants may comprise from about 0.25 wt % to about 10 wt % or from about 0.5 wt % to about 3 wt % of the tablet. Tablet blends may be compressed directly or by roller compaction to form tablets. Tablet blends or portions of blends may alternatively be wet-, dry-, or melt-granulated, melt congealed, or extruded before tableting. If desired, prior to blending one or more of the components may be sized by screening or milling or both. The final dosage form may comprise one or more layers and may be coated, uncoated, or encapsulated. Exemplary tablets may contain up to about 80 wt % of API, from about 10 wt % to about 90 wt % of binder, from about 0 wt % to about 85 wt % of diluent, from about 2 wt % to about 10 wt % of disintegrant, and from about 0.25 wt % to about 10 wt % of lubricant.

Consumable oral films for human or veterinary use are pliable water-soluble or water-swellable thin film dosage forms which may be rapidly dissolving or mucoadhesive. In addition to the active pharmaceutical agent, a typical film includes one or more film-forming polymers, binders, solvents, humectants, plasticizers, stabilizers or emulsifiers, viscosity-modifying agents, solvents and other ingredients. If water soluble, the API would typically comprise from about 1 wt % to about 80 wt % of the non-solvent components (solutes) in the film or from about 20 wt % to about 50 wt % of the solutes in the film. A less soluble API may comprise a greater proportion of the composition, typically up to about 88 wt % of the non-solvent components in the film.

The film-forming polymer may be selected from natural polysaccharides, proteins, or synthetic hydrocolloids and typically comprises from about 0.01 wt % to about 99 wt % or from about 30 wt % to about 80 wt % of the film. Film dosage forms are typically prepared by evaporative drying of thin aqueous films coated onto a peelable backing support or paper, which may carried out in a drying oven or tunnel (e.g., in a combined coating-drying apparatus), in lyophilization equipment, or in a vacuum oven.

Useful solid formulations for oral administration may include immediate release formulations and modified release formulations. Modified release formulations include delayed-, sustained-, pulsed-, controlled-, targeted-, and programmed-release. Compounds herein, and the pharmaceutically acceptable salts thereof, may also be administered directly into the blood stream, muscle, or an internal organ of the subject. Suitable parenteral administrations include intravenous, intraarterial, intraperitoneal, intrathecal, intraventricular, intraurethral, intrasternal, intracranial, intramuscular, intrasynovial, and subcutaneous administration via needle injectors, microneedle injectors, needle-free injectors, and infusion devices.

The compounds herein, and the pharmaceutically acceptable salts thereof, may also be administered topically, intradermally, or transdermally to the skin or mucosa. Typical formulations for this purpose include gels, hydrogels, lotions, solutions, creams, liposomes, ointments, dusting powders, dressings, foams, films, skin patches, wafers, implants, sponges, fibers, bandages and microemulsions using carriers and methods known in the art.

The compounds herein, and the pharmaceutically acceptable salts thereof, may also be administered intranasally or by inhalation, typically in the form of a dry powder, an aerosol spray, or nasal drops. The active compounds may also be administered rectally or vaginally, e.g., in the form of a suppository, pessary, or enema.

In the case of dry powder inhalers and aerosols, the dosage unit is determined by means of a valve that delivers a metered amount. Units are typically arranged to administer a metered dose or “puff' containing from about 10 μg to about 1000 μg of the API. The overall daily dose will typically range from about 100 μg to about 10 mg which may be administered in a single dose or, more usually, as divided doses throughout the day.

As noted above, the compounds herein, and the pharmaceutically acceptable salts thereof, and their pharmaceutically active complexes, solvates and hydrates, may be combined with one another or with one or more other active pharmaceutically active compounds to treat various diseases, conditions and disorders. In such cases, the active compounds may be combined in a single dosage form as described above or may be provided in the form of a kit which is suitable for coadministration of the compositions.

For administration to human patients, the total daily dose of the claimed and disclosed compounds is typically in the range of about 0.1 mg to about 3000 mg depending on the route of administration. For example, oral administration may require a total daily dose of from about 1 mg to about 3000 mg, while an intravenous dose may only require a total daily dose of from about 0.1 mg to about 300 mg. The total daily dose may be administered in single or divided doses and, at the physician's discretion, may fall outside of the typical ranges given above. Although these therapeutically effective dosages are based on an average human subject having a mass of about 60 kg to about 70 kg, the physician will be able to determine the appropriate dose for a patient (e.g., an infant) whose mass falls outside of this weight range.

The claimed and disclosed compounds may be combined with one or more other pharmacologically active compounds for the treatment of one or more related disorders, the pharmacologically active compounds can be selected from: 1) an opioid analgesic, e.g. morphine, fentanyl, codeine, etc.; 2) a nonsteroidal antiinflammatory drug (NSAID), e.g. acetaminophen, aspirin, diclofenac, etodolac, ibuprofen, naproxen, etc.; 3) a barbiturate sedative, e.g. pentobarbital; 4) a benzodiazepine having a sedative action, e.g. diazepam, lorazepam, etc.; 5) an H₁ antagonist having a sedative action, e.g. diphenhydramine; 6) a sedative such as glutethimide, meprobamate, methaqualone or dichloralphenazone; 7) a skeletal muscle relaxant, e.g. baclofen, carisoprodol, chlorzoxazone, cyclobenzaprine, methocarbamol or orphrenadine; 8) an NMDA receptor antagonist; 9) an alpha-adrenergic; 10) a tricyclic antidepressant, e.g. desipramine, imipramine, amitriptyline or nortriptyline; 11) an anticonvulsant, e.g. carbamazepine, lamotrigine, topiratmate or valproate; 12) a tachykinin (NK) antagonist, particularly an NK-3, NK-2 or NK-1 antagonist; 13) a muscarinic antagonist, e.g oxybutynin, tolterodine, etc.; 14) a COX-2 selective inhibitor, e.g. celecoxib, valdecoxib, etc.; 15) a coal-tar analgesic, in particular paracetamol; 16) a neuroleptic such as haloperidol, clozapine, olanzapine, risperidone, ziprasidone, or Miraxion®; 17) a vanilloid receptor (VR1; also known as transient receptor potential channel, TRPV1) agonist (e.g. resinferatoxin) or antagonist (e.g. capsazepine); 18) a beta-adrenergic such as propranolol; 19) a local anaesthetic such as mexiletine; 20) a corticosteroid such as dexamethasone; 21) a 5-HT receptor agonist or antagonist, particularly a 5-HT_{1B/1D} agonist such as eletriptan, sumatriptan, naratriptan, zolmitriptan or rizatriptan; 22) a 5-HT_{2A} receptor antagonist such as R(+)-alpha-(2,3-dimethoxy-phenyl)-1-[2-(4-fluorophenylethyl)]-4-piperidinemethanol (MDL-100907); 23) a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine, or a nicotine partial agonist such as varenicline; 24) Tramadol®; 25) a PDEV inhibitor; 26) an alpha-2-delta ligand such as gabapentin, pregabalin, 3-methylgabapentin, etc.; 27) a cannabinoid receptor (CB1, CB2) ligand, either agonist or antagonist such as rimonabant; 28) metabotropic glutamate subtype 1 receptor (mGluR1) antagonist; 29) a serotonin reuptake inhibitor such as sertraline, sertraline metabolite demethylsertraline, fluoxetine, etc.; 30) a noradrenaline (norepinephrine) reuptake inhibitor, such as buproprion, buproprion metabolite hydroxybuproprion, especially a selective noradrenaline reuptake inhibitor such as reboxetine, in particular (S,S)-reboxetine; 31) a dual serotonin-noradrenaline reuptake inhibitor, such as venlafaxine, O-desmethylvenlafaxine, clomipramine, desmethylclomipramine, duloxetine, milnacipran and imipramine; 32) an inducible nitric oxide synthase (iNOS) inhibitor; 33) an acetylcholinesterase inhibitor such as donepezil; 34) a prostaglandin E₂ subtype 4 (EP4) antagonist; 35) a leukotriene B4 antagonist; 36) a 5-lipoxygenase inhibitor, such as zileuton; 37) a sodium channel blocker, such as lidocaine; 38) a 5-HT3 antagonist, such as ondansetron; or 39) anti-nerve growth factor (NGF) antibodies. It is understood that the pharmaceutical agents just mentioned may be administered in the manner and at the dosages known in the art.

The compounds described herein (including the precursor intermediates) can have one or more chiral centers and one or more alkenyl moieties. Where the synthesis yields a compound as a mixture of isomers (e.g., enantiomers, diastereomers, and/or geometric isomers), the desired isomer (or the desired enantiomerically-, diastereomerically-, or geometrically-enriched mixture) can be obtained using conventional chiral resolution methods including chromatography (such as HPLC) or supercritical fluid chromatography (SFC) on an asymmetric resin, such as Chiralcel OJ-H, Chiralpak AD-H, Chiralpak IA and Chiralpak AS-H brand chiral stationary phases available from Deicel Chemical Industries, Ltd, Japan, with a mobile phase typically comprising an alcohol (e.g., from about 10% to about 50% by volume) and carbon dioxide. Concentration of the eluate affords the isomerically enriched mixture, which may also be further derivatized.

The compounds herein, and the pharmaceutically acceptable salts thereof, may be generally prepared using the techniques described below. Starting materials and reagents may be obtained from commercial sources or may be prepared using literature methods unless otherwise specified. In some of the reaction schemes and examples below, certain compounds can be prepared using protecting groups, which prevent undesirable chemical reaction at otherwise reactive sites. Protecting groups may also be used to enhance solubility or otherwise modify physical properties of a compound. A discussion of protecting group strategies can be seen in T. W. Greene and P. G. Wuts, Greene's Protective Groups in Organic Chemistry (4^{th} Ed., 2007) and P. Kocienski, Protective Groups (2000).

Generally, the chemical reactions described throughout the specification may be carried out using substantially stoichiometric amounts of reactants, though certain reactions may benefit from using an excess of one or more of the reactants. Additionally, many of the reactions disclosed throughout the specification may be carried out at about room temperature and ambient pressure, but depending on reaction kinetics, yields, and the like, some reactions may be run at elevated pressures or employ higher (e.g., reflux conditions) or lower (e.g., −70° C. to 0° C.) temperatures. Any reference in the disclosure to a stoichiometric range, a temperature range, a pH range, etc., whether or not expressly using the word “range,” also includes the indicated endpoints.

Many of the chemical reactions may also employ one or more compatible solvents, which may influence the reaction rate and yield. Depending on the reactants, the one or more solvents may be polar protic solvents (including water), polar aprotic solvents, non-polar solvents, or some combination. Representative solvents include saturated aliphatic hydrocarbons (e.g., n-pentane, n-hexane, n-heptane, n-octane); aromatic hydrocarbons (e.g., benzene, toluene, xylenes); halogenated hydrocarbons (e.g., methylene chloride (DCM), chloroform, carbon tetrachloride); aliphatic alcohols (e.g., methanol (MeOH), ethanol (EtOH), propan-1-ol, propan-2-ol (IPA), butan-1-ol, 2-methyl-propan-1-ol, butan-2-ol, 2-methyl-propan-2-ol, pentan-1-ol, 3-methyl-butan-1-ol, hexan-1-ol, 2-methoxy-ethanol, 2-ethoxy-ethanol, 2-butoxy-ethanol, 2-(2-methoxy-ethoxy)-ethanol, 2-(2-ethoxy-ethoxy)-ethanol, 2-(2-butoxy-ethoxy)-ethanol); ethers (e.g., diethyl ether, di-isopropyl ether, dibutyl ether, 1,2-dimethoxy-ethane (DME), 1,2-diethoxy-ethane, 1-methoxy-2-(2-methoxy-ethoxy)-ethane, 1-ethoxy-2-(2-ethoxy-ethoxy)-ethane, tetrahydrofuran (THF), 1,4-dioxane); ketones (e.g., acetone, methyl ethyl ketone (MEK)); esters (methyl acetate, ethyl acetate (EA or EtOAc); nitrogen-containing solvents (e.g., formamide, N,N-dimethyl formamide (DMF), acetonitrile, N-methyl-pyrrolidone (NMP), pyridine, quinoline, nitrobenzene); sulfur-containing solvents (e.g., carbon disulfide, dimethyl sulfoxide (DMSO), tetrahydro-thiophene-1,1,-dioxide); and phosphorus-containing solvents (e.g., hexamethylphosphoric triamide).

The compounds herein may be prepared as described below. In the reaction schemes and discussion that follow, Ar¹, Ar², R¹, R², and R³ are defined as above. Furthermore, Ar¹ and Ar² may be substituted as defined above.

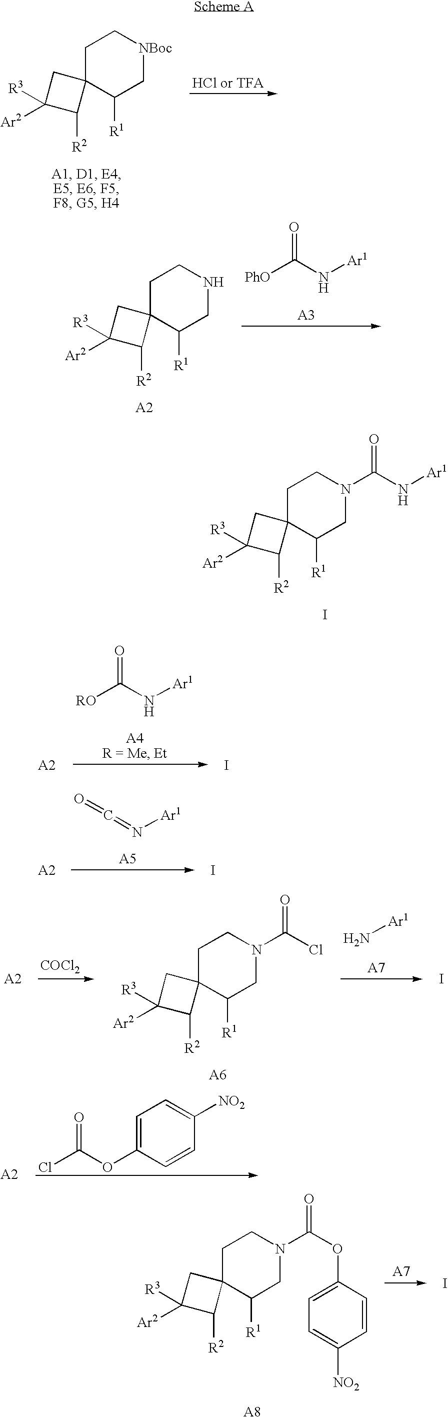

Compounds of Formula I can be prepared according to Scheme A. Compounds of formula A1, D1, E4, E5, E6, F5, F8, G5 and H4 can be deprotected using conventional methods (for example, using HCl/dioxane in dichloromethane, acetyl chloride in ethanol, or trifluoroacetic acid (TFA) in dichloromethane) to provide the corresponding compounds of formula A2 which can be isolated as the free base or as the corresponding salt (hydrochloride or trifluoroacetate). The reaction of a compound of formula A2 with a phenyl carbamate of formula A3 provides compounds of the Formula I. The reaction can be conducted in a polar aproptic solvent such as DMSO or acetonitrile. The temperature of the reaction may vary from about ambient temperature to about 60° C. The reaction can also be conducted using a trifluoroacetate or hydrochloride salt of the compound of formula A2 in the presence of a base such as triethylamine (TEA) or diisopropylethyl amine (DIEA). Alternatively, the reaction of a compound of formula A2 with a carbamate of formula A4 (R=Me or Et) under microwave irradiation may provide compounds of the Formula I. The reaction may be conducted in a solvent such as acetonitrile. The reaction may also be conducted using a trifluoroacete or hydrochloride salt of the compound of formula A2 in the presence of a base such as TEA or DIEA. Furthermore, compounds of the Formula I may be prepared by reacting compounds of formula A2 with an isocyanate of formula A5. The reaction may be conducted in a solvent such as dichloromethane at ambient temperature. The reaction may also be conducted using a trifluoroacetate or hydrochloride salt of the compound of formula A2 in the presence of a base such as TEA or DIEA. Alternatively, compounds of formula A2 may be reacted with phosgene in the presence of a base such as TEA or DIEA and a solvent such as dichloromethane at about 0° C. to generate compounds of formula A6 which may be isolated as a crude material and reacted with aryl amines of formula A7 in the presence of a base such as TEA or DIEA and a catalyst such as 4-(dimethylamino)-pyridine (DMAP) in a suitable solvent such as acetonitrile, dichloromethane, and dichloroethane. The reaction temperature may vary from about ambient temperature to about 70° C. Alternatively, compounds of formula A2 may be reacted with 4-nitrophenyl chloroformate in the presence of a base such as aqueous sodium bicarbonate and a solvent such as dioxane at room temperature generate compounds of formula A8 which may be isolated as a crude material, optionally purified, and reacted with aryl amines of formula A7 in the presence of a base such as sodium hydride in a suitable solvent such as DMF or DMA. The reaction temperature may vary from about ambient temperature to about 70° C.

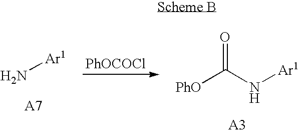

Scheme B illustrates a method for making phenyl carbamates of formula A3. Treatment of an aryl amine of formula A7 with phenyl chloroformate in a solvent such as THF, DCM, 1,4-dioxane, acetonitrile, DMF, or DMSO gives phenyl carbamates of formula A3 in a manner similar to that described in Synthesis, 1997, 1189-1194. The reaction may be performed in the presence of a base such as TEA, DIEA, 1,8-bis(dimethylamino)naphthalene (Proton Sponge®), and the like. The temperature of the reaction may vary from about 0° C. to reflux temperature of the solvent being used.

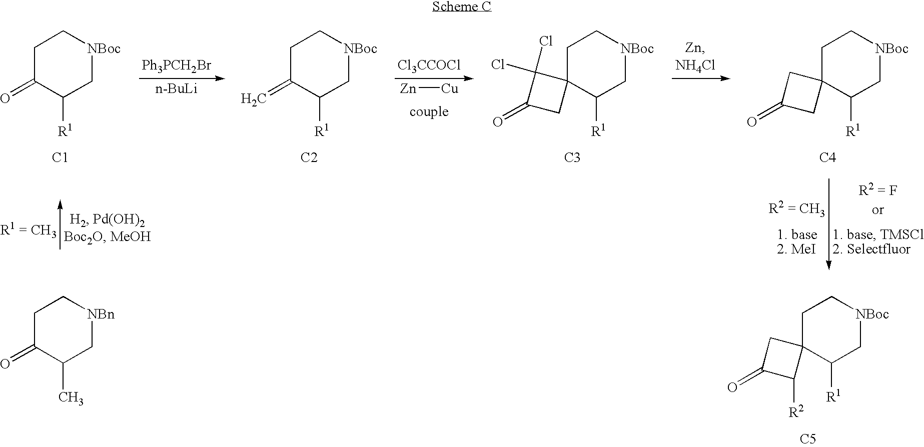

Ketone intermediates of formulae C4 and C5 can be prepared according to Scheme C. A compound of formula C1 (e.g., tert-butyl 4-oxopiperidine-1-carboxylate (CAS #79099-07-3), tert-butyl 3-fluoro-4-oxopiperidine-1-carboxylate (CAS #211108-50-8; van Niel et al. J. Med. Chem., 1999, 42, 2087-2104), or tert-butyl 3-methyl-4-oxopiperidine-1-carboxylate (CAS #181269-69-2) which can be prepared from 1-benzyl-3-methyl-piperidin-4-one (CAS #34737-89-8) as described by Luly et al. US 2005/0070549, Mar. 31, 2005) may be converted to an olefin of formula C2 in a manner similar to that described by Ting et al. US 2005/0182095, Aug. 18, 2005. Olefins of formula C2 may be reacted with dichloroketene (generated in situ from excess trichloroacetyl chloride in the presence of excess zinc-copper couple obtained from Alfa-Aesar) to give compounds of formula C3 in a manner similar to that described by Kaneko et al. Chem. Pharm. Bull. 2004, 52, 675-687. The reaction is preferably performed in an ethereal solvent such as DME at a temperature ranging from about 30° C. to 45° C. Compounds of formula C3 can be preferably reduced in the presence of fresh zinc dust and ammonium chloride in a solvent such as methanol to furnish compounds of formula C4 in a manner similar to that described by Kaneko et al. Chem. Pharm. Bull. 2004, 52, 675-687. Alternatively, compounds of formula C3 can be reduced in the presence of hydrogen at about atmospheric pressure to 10 psi in the presence of a catalyst such as 5% palladium on carbon in the presence of a base such as pyridine and solvents such as ethyl acetate and water to furnish compounds of formula C4 in a manner similar to that described by Takuma et al. JP2002-249454. Compounds of formula C4 can be further elaborated by lithiation with a strong base such as lithium diisopropylamide (LDA) or lithium hexamethyldisilazide (LHMDS) and reaction with an alkylating agent such as iodomethane in a solvent such as THF at a temperature ranging from −78° C. to room temperature to provide compounds of formula C5 (R²═CH₃). Alternatively, compounds of formula C4 may be further elaborated by lithiation with a strong base such as LDA or LHMDS, trapped as the silyl enolate with trimethylsilylchloride (TMSCl), and reaction with a fluorinating agent such as Selectfluor® (CAS #140681-54-5) in a solvent such as THF to provide compounds of formula C5 (R²═F).

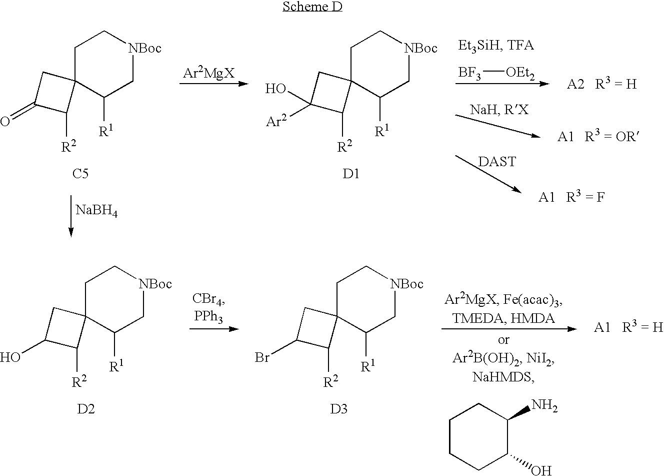

Compounds of formula A1 and A2 can be prepared according to Scheme D. Aryl Grignard reagents (Ar²MgX; X═Cl, Br, or I) can be purchased commercially or prepared from an aryl halide with reagents such as magnesium (for a review see Lai, Y. H. Synthesis 1981, 585-604) or isopropylmagnesium chloride (for a review see P. Knochel et al. Angew. Chem. Int. Ed. 2003, 42, 4302-4320; for the use of lithium chloride as an additive, see Krasovskiy and Knochel, Angew. Chem. Int. Ed. 2004, 43, 3333-3336). Addition of an aryl Grignard (Ar²MgX) to ketone compounds of formula C5 in a solvent such as THF at 0° C. to about room temperature gives alcohol compounds of formula D1. Alcohols of formula D1 can be treated with triethylsilane, trifluoroacetic acid, and boron trifluoride-diethyl etherate in a solvent such as dichloromethane at about −15° C. to about room temperature to give the reduced compounds of formula A2 (R³═H). Furthermore, compounds of formula D1 can also be alkylated with a base such as sodium hydride and an alkyl halide R′X (X═Br or I) in a solvent such as DMF or DMA to provide the corresponding compounds of formula A1 (R³═OR′). Additionally, compounds of formula D1 can also be treated with diethylaminosulfur trifluoride (DAST) in a solvent such as dichloromethane at −78° C. to about 0° C. to provide the corresponding compounds of formula A1 (R³═F). Compounds of formula C5 can also be reacted with a reducing agent such as sodium borohydride in methanol to give alcohols of formula D2, which can be converted to bromides of formula D3 with triphenylphosphine and carbon tetrabromide in a solvent such as THF. Compounds of formula D3 can be coupled with aryl Grignard reagents (Ar²MgX; X═Cl, Br, I) in the presence of catalytic amounts of Fe(acac)₃, tetramethylethylenediamine (TMEDA) and hexamethylenetetramine (HMTA) in THF in a manner similar to that described by Cahiez et al., Angew. Chem. Int. Ed. 2007, 46 4364-4366, to give compounds of formula A1 (R³═H). Alternatively, compounds of formula D3 can be coupled with aryl boronic acids (Ar²B(OH)₂) in the presence of sodium hexamethyldisilazide (NaHMDS) and catalytic amounts of nickel iodide and trans-2-aminocyclohexanol in anhydrous isopropanol in a manner similar to that described by Gonzalez-Bobes and Fu, J. Am. Chem. Soc. 2006, 128, 5360-5361, to give compounds of formula A1 (R³═H).

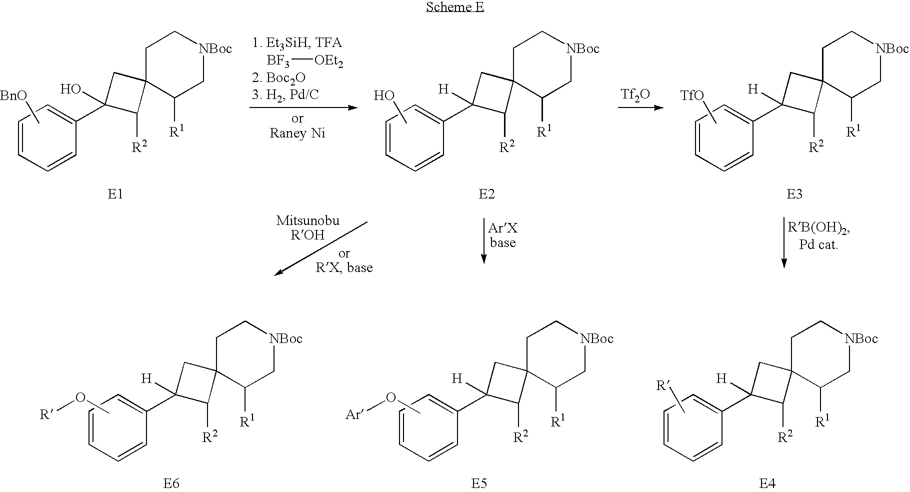

Compounds of formulae E4-E6 can be prepared according to Scheme E. Compounds of formula E1 can be prepared as described in Scheme D for compounds of formula D1 (Ar²=2-, 3-, or 4-benzyloxyphenyl). Compounds of formula E1 can be reduced by treatment with triethylsilane, TFA, and boron trifluoride-diethyl etherate as described for Scheme D, followed by reprotection of the amine with di-tert-butyl dicarbonate in dichloromethane in the presence of a base such as triethylamine. Finally, treatment with catalytic palladium on carbon under an atmosphere of hydrogen at about 10 to about 50 psi can give compounds of formula E2. Alternatively, compounds of formula E1 can be converted directly to compounds of formula E2 using excess Raney nickel in a solvent such as ethanol at reflux. Compounds of formula E2 can be treated with triflic anhydride in a solvent such as dichloromethane and the presence of a base such as pyridine to give compounds of formula E3. Triflates of formula E3 can be reacted with an aryl or alkyl boronic acid of formula (R′B(OH)₂) under palladium-catalyzed Suzuki cross-coupling conditions (for a review, see Chem. Rev. 1995, 95, 2457), to give the corresponding compounds of formula E4. For example, the coupling can be conducted using a catalytic amount of tetrakis(triphenylphosphine)-palladium(0) in the presence of a base such as aqueous sodium carbonate, cesium carbonate, sodium hydroxide, or sodium ethoxide, in a solvent such as THF, dioxane, ethylene glycol dimethylether, DMF, ethanol or toluene. The temperature of the reaction may vary from about ambient temperature to about the reflux temperature of the solvent used. Further, compounds of formula E5 can be prepared by a nucleophilic aromatic substitution of a phenol of formula E2 with an electron deficient aryl halide (Ar′X; X═Cl or F) to form the biaryl ether of formula E5. This reaction is preferably run in the presence of a base such as potassium carbonate, sodium carbonate, cesium carbonate, NaHMDS, triethylamine or diisopropylethylamine. The solvent used may be DMF, DMA, NMP, DMSO, acetonitrile, tetrahydrofuran, dioxane or a combination of two or more of these solvents. Further, phenol compounds of formula E2 can be alkylated with an an alkyl halide (R′X; X═Cl, Br or I) using a base such as cesium carbonate, potassium carbonate, or sodium hydride in a solvent such as DMF, DMA, NMP, DMSO, dioxane, or acetonitrile, to yield compounds of formula E6. The temperature of the reaction may vary from about ambient temperature to about the reflux temperature of the solvent used and may be heated under conventional or microwave conditions. Sodium iodide or potassium iodide may be added to facilitate the alkylation. Alternatively, the phenol of compounds E2 can be reacted with alkyl alcohols (R′OH) under Mitsunobu reaction conditions (Organic Reactions 1992, 279, 22-27; Org. Prep. Proc. Int. 1996, 28, 127-164; Eur. J. Org. Chem. 2004, 2763-2772) such as polystyrene-triphenylphosphine (PS-PPh₃) and di-tert-butyl azodicarboxylate (DBAD) to give compounds of formula E6.

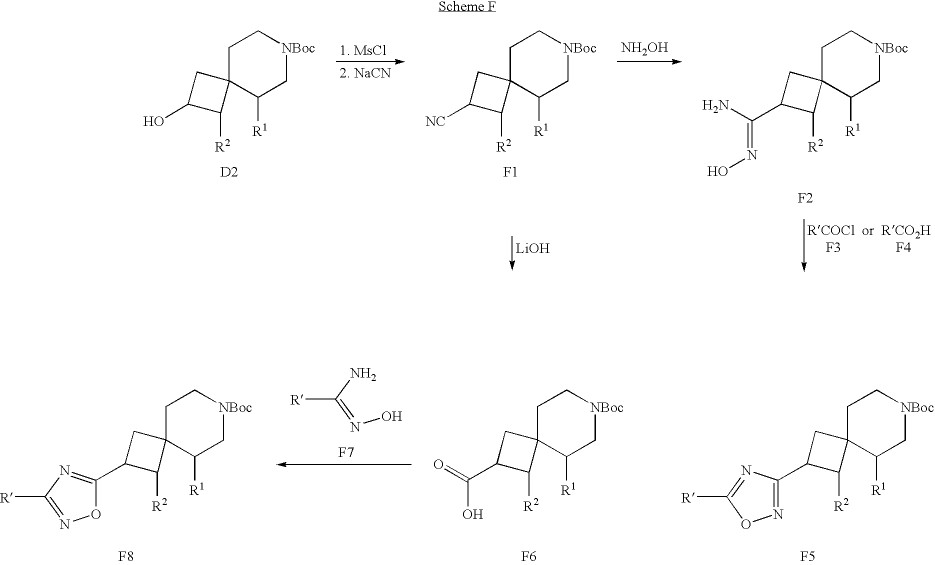

Compounds of formulae F5 and F8 can be prepared according to Scheme F. Alcohols of formula D2 can be treated with methanesulfonyl chloride in a solvent such as dichloromethane in the presence of a base such as triethylamine or DIEA. The meslyate intermediate can then be reacted with sodium cyanide in a suitable solvent such as DMF or DMSO at a temperature ranging from room temperature to about 90° C. to give nitrile compounds of formula F1. Nitriles of formula F1 can be treated with excess hydroxylamine hydrochloride and TEA in a solvent such as ethanol. The reaction is run at about 80° C. to reflux temperature of the solvent used to give hydroxyamidines of formula F2. Hydroxyamidines of formula F2 can be treated with acid chlorides of formula F3 in a solvent such as THF and the presence of a base such as DIEA or TEA. The reaction can be run at reflux of the solvent used and may be heated by conventional or microwave conditions to give oxadiazoles of formula F5. Alternatively, hydroxyamidines of formula F2 may be reacted with carboxylic acids of formula F4 in the presence of a coupling agent such as carbonyldiimidazole (CDI), O-(Benzotriazol-1-yl)-N,N,N′,N′-tetramethyluronium hexafluorophosphate (HBTU), and the like, in a solvent such as DMF in the presence of a base such as TEA or DIEA. The reaction may be run at room temperature followed by heating to about 110° C. to give oxadiazole compounds of formula F5. Nitriles of formula F1 can also be hydrolyzed by treatment with lithium hydroxide in a solvent such as ethanol/water at about reflux temperature to give carboxylic acids of formula F6. Carboxylic acids of formula F6 may then be converted to their acid chloride with thionyl chloride or oxalyl chloride and reacted with hydroxyamidines of formula F7 as described above to give oxadiazoles of formula F8. Alternatively, reactions of carboxylic acids of formula F6 with coupling agents such as CDI or HBTU and hydroxyamidines of formula F7 as described above to give oxadiazoles of formula F8.

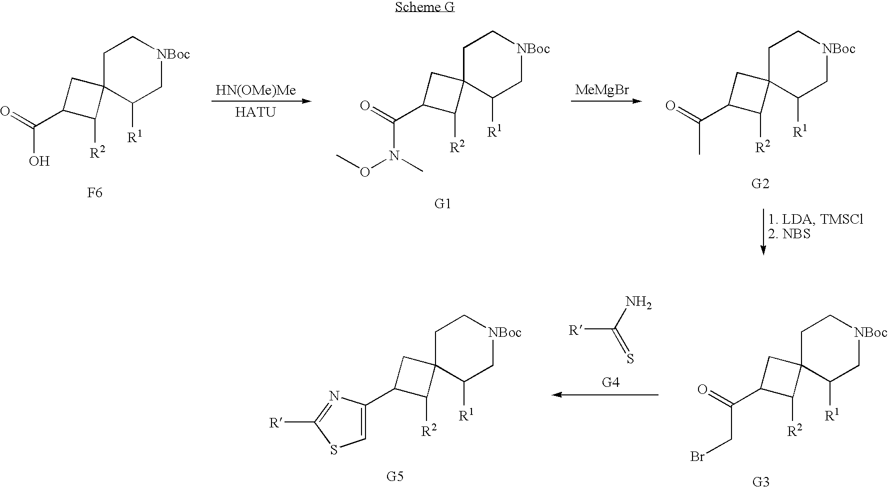

Thiazole compounds of formula G5 can be prepared according to Scheme G. Compounds of formula F6 can be treated with N,O-dimethylhydroxylamine hydrochloride in the presence a coupling agent such as O-(7-azabenzotriazol-1-yl)-N,N,N′,N′-tetramethyluronium hexafluorophosphate (HATU), and a base such as DIEA or TEA in a solvent such as dichloromethane to give the Weinreb amide of formula G1. The compound of formula G1 can be treated with methyl magnesium bromide in a solvent such as THF at about 0° C. to room temperature to give methyl ketone compounds of formula G2. Compounds of formula G2 can be treated with LDA in a solvent such as THF at about −78° C. followed by treatment with trimethylsilyl chloride (TMSCl). After isolation, the silyl enolate intermediate can be treated with sodium bicarbonate in THF followed by N-bromosuccinimide (NBS) at 0° C. to give α-bromoketone compounds of formula G3. Compounds of formula G3 can be reacted with thioamides of formula G4 in a solvent such as ethanol at a temperature ranging from about 80° C. to reflux temperature of the solvent used to give thiazole compounds of formula G5.

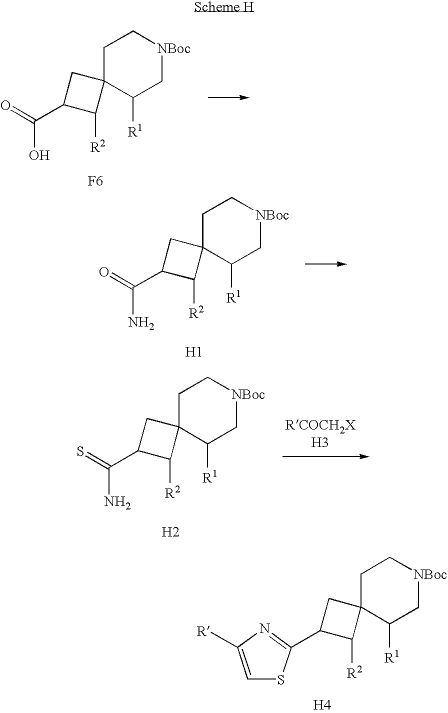

Thiazole compounds of formula H4 can be prepared according to Scheme H. Carboxylic acid compounds of formula F6 can be treated with ammonia in methanol in the presence a coupling agent such as HATU, and a base such as DIEA or TEA in a solvent such as dichloromethane to give the carboxamide of formula H1. Compounds of formula H1 can be treated with Lawesson's reagent in a solvent such as toluene. The reaction may be heated to about 65° C. to reflux temperature of the solvent used to provide thioamides of formula H2. Thioamides of formula H2 may be treated with α-haloketones of formula H3 (X═Cl or Br) in a solvent such as ethanol as described for Scheme G to give thiazole compounds of formula H4.

### Examples

The following examples are intended to illustrate particular aspects of the compounds and methods described herein and are not intended to limit the scope of the claims.

¹H Nuclear magnetic resonance (NMR) spectra were obtained for the compounds in the following examples. Characteristic chemical shifts (δ) are given in parts-per-million (ppm) downfield from tetramethylsilane using conventional abbreviations for designation of major peaks, including s (singlet), d (doublet), t (triplet), q (quartet); m (multiplet), and br (broad). The following abbreviations are used for common solvents: CDCl₃ (deuterochloroform), DMSO-d₆ (deuterodimethylsulfoxide), and methanol-d₆ (deuteromethanol). Liquid chromatography-mass spectrometry (LCMS) were recorded using electrospray (ES) or atmospheric pressure chemical ionization (APCI) techniques.

### Synthesis of tert-butyl 4-methylenepiperidine-1-carboxylate

A reactor was charged with THF (12.2 L) and methyl phosphonium bromide (1997 g, 5.59 mol) and cooled to −40° C. A solution of n-butyllithium (2.6 M in THF; 2.03 L, 5.28 mol) was added slowly to the mixture, maintaining a temperature below −45° C. The mixture was warmed to −20° C. for 1 h, then cooled to −70° C. and treated dropwise with a solution of tert-butyl 4-oxopiperidine-1-carboxylate (747 g, 3.75 mol; CAS #79099-07-3) in THF (2.69 L) over 30 min, maintaining a temperature below −55° C. The reaction mixture was warmed to ambient temperature with stirring. The mixture was transferred to a 50 L reactor and treated with cyclohexane (10 L) and water (10 L). After mixing, the layers were separated, and the organic layer was washed with brine (10 L). The organic layer was concentrated to give an oil which was dissolved in diethyl ether (3 L), cooled to 0° C., and filtered to remove triphenylphosphine waste. The filtrate was purified by filtration through a 4 kg plug of silica gel in 80:20 hexane:ethyl acetate to give 667 g of the crude title compound (˜90% pure by TLC). The crude was purified by short path distillation using a wiped film evaporator at 90° C. to yield the title compound (599 g, 81%). ¹H NMR (400 MHz, CDCl₃) δ ppm 4.72 (2H, s), 3.41-3.38 (4H, t, J=5.64 Hz), 2.17-2.14 (4H, t, J=5.2 Hz), 1.45 (9H, s); GCMS m/z 197.

### Synthesis of tert-butyl 1,1-dichloro-2-oxo-7-azaspiro[3.5]nonane-7-carboxylate

Dry DME (8.0 L) and tert-butyl 4-methylenepiperidine-1-carboxylate (800 g, 4.06 mol) were charged to a reactor. Zinc-copper couple (800 g; CAS #53801-63-1, Alfa-Aesar) was charged to the reactor, and the mixture was warmed to 34° C. Trichloroacetyl chloride (1448 g, 8.0 mol, 888 mL) was added dropwise under a nitrogen atmosphere to the stirred suspension in the following manner: 80 mL of trichloroacetyl chloride was added. After 10 min, an exotherm elevated the reaction temperature to 39° C. Dropwise addition of the remaining trichloroacetyl chloride was resumed immediately at a rate to maintain a temperature between 40-44° C. using a 25° C. jacket. After the addition was complete, the reaction was stirred at 40° C. for 15 min. Cyclohexane (10 L) was added to the mixture. The mixture was filtered through a pad of celite, washing with cyclohexane (2 L). The filtrate was concentrated to approximately 3 L and then was diluted with MTBE (3 L) and cyclohexane (2 L) and filtered through a pad of magnesol (1 kg), washing with 1:1 cyclohexane/MTBE (3 L). The filtrate was washed with saturated potassium bicarbonate (3 L) and brine (2 L). The organic layer was filtered through a pad of silica gel (300 g) with a pad of magnesol (200 g) on top. The filtrated was concentrated to yield the title compound as an orange solid (1123 g, 91%). ¹H NMR (400 MHz, CDCl₃) δ ppm 4.05-4.13 (m, 2H), 3.08 (s, 2H), 2.80-2.88 (m, 2H), 1.88-1.97 (m, 2H), 1.71-1.78 (m, 2H), 1.46 (s, 9H). m/z 252, 254 (MH⁺ minus t-Bu).

### Synthesis of tert-butyl 2-oxo-7-azaspiro[3.5]nonane-7-carboxylate

Method A. A mixture of ammonium chloride (832 g, 15 mol) and methanol (11 L) in a 20 L reactor was stirred and cooled to 0° C. A solution of tert-butyl 1,1-dichloro-2-oxo-7-azaspiro[3.5]nonane-7-carboxylate (1393 g, 4.5 mol) in methanol (2.5 L) was added to the mixture, followed by a 500 mL methanol wash. The mixture was cooled to 0° C. and treated with zinc dust (1400 g) in 50 g portions, keeping the reaction temperature below 8° C. with 0° C. cooling. After the first 250 g of zinc was added, the jacket temperature was raised to 12° C., and the next 500 g of zinc was added in 100 g portions over two hours. The reaction temperature was raised to 15° C. and the remaining 650 g of zinc was added in 100 g portions over 1 h. The temperature was raised to 25° C. and treated with an additional 472 g of zinc. The reaction was stirred at 30° C. for 1 h. The mixture was filtered through a pad of celite, washing with methanol (2 L). The filtrate was concentrated to ˜1.2 L and diluted with MTBE (3 L). The organic was extracted with saturated ammonium chloride solution (2×1 L) and brine (1 L). The organic layer was filtered through magnesol (1 kg), washing with MTBE (2 L). The filtrate was concentrated to give a yellow oil (870 g) which was dissolved in hexane (2 L), cooled to 0° C., and filtered, washing with cold hexane (1 L) to give the title compound (740 g). The filtrate was concentrated to give an oil (130 g) which was combined with additional product (83 g) from re-extractions from the aqueous phases with MTBE (2 L) which were passed through the same magnesol cake with MTBE (2 L). The combined 213 g of oil was purified by short path distillation using a wiped film evaporator at 130° C. and 500 mtorr to yield 145 g which was crystallized from hexane to give 120 g of the title compound. Combination of the 740 g and 120 g batches gave the title compound as a white solid (860 g, 80%). ¹H NMR (400 MHz, CDCl₃) δ ppm 3.40-3.37 (4H, t, J=5.44 Hz), 2.8 (4H, s), 1.69-1.67 (4H, t, J=5.36 Hz), 1.45 (9H, s); GCMS m/z 239.

Method B. A mixture of tert-butyl 1,1-dichloro-2-oxo-7-azaspiro[3.5]nonane-7-carboxylate (18.4 g, 59.4 mmol), 5% Pd/C (9 g), pyridine (18 mL), EtOAc (360 mL), and water (180 mL) was stirred under an atmosphere of hydrogen (balloon) for 3 days. The reaction was monitored by ¹H NMR. The reaction mixture was degassed and back flushed with nitrogen. The mixture was filtered over Celite and the aqueous layer was removed from the filtrate. The organic layer was washed with brine and water, dried over sodium sulfate, filtered, and concentrated. The residue was dissolved in methylene chloride and purified by flash chromatography (silica gel, 20% ethyl acetate/hexanes, fractions identified by staining TLC with iodine) to give the title compound as a white solid (8.0 g, 56%).

### Synthesis of tert-butyl 2-[3-(benzyloxy)phenyl]-7-azaspiro[3.5]nonane-7-carboxylate

To a solution of tert-butyl 2-oxo-7-azaspiro[3.5]nonane-7-carboxylate (20.0 g, 83.6 mmol) in 2-MeTHF (300 mL) at 0° C. was added 3-benzyloxyphenylmagnesium bromide (1.0 M in THF, 100 mL, 100 mmol, 1.2 equiv; Aldrich) dropwise via addition funnel at a rate such that the reaction temperature did not exceed 5 C (approx. 25 min). The reaction was stirred at 0° C. for 1 h and treated another 10 mL of 3-benzyloxyphenylmagnesium bromide (1.0 M in THF). After 30 min at 0° C., the reaction was quenched with satd ammonium chloride. The organic layer was washed with saturated ammonium chloride. The aq layer was extracted with ethyl acetate. The organic layers were combined, washed with brine, dried over sodium sulfate, filtered, and concentrated to give the crude alcohol (41.3 g). A solution of the crude alcohol and triethylsilane (66.7 mL, 418 mmol) in methylene chloride (350 mL) was treated with boron trifluoride diethyl etherate (20.6 mL, 167 mmol) and trifluoroacetic acid (31.0 mL, 418 mmol) at 0° C. After 1 h, the reaction was quenched with 3 N HCl. The organic layer was washed with water and satd sodium bicarbonate. The organic layers were dried over sodium sulfate, filtered, and concentrated. The residue was resuspended in ethyl acetate and washed with water to remove some insoluble gum. The organic layer was washed with brine, dried, and concentrated to give the crude amine (33.5 g). Di-tert-butyl dicarbonate (20.0 g, 91.6 mmol; CAS #24424-99-5) was added to a solution of the crude amine in dichloromethane (400 mL) at room temp, followed by triethylamine (15.0 mL, 108 mmol). After 1 h, reaction was washed with water and the organic phase was dried over magnesium sulfate and filtered. The filtrate was treated with 85 g silica gel and concentrated to dryness. The compound/silica gel mixture was purified by flash chromatography (0 to 15% ethyl acetate/heptane) to give the title compound as a waxy white solid (13.1 g, 38.5%). m/z 430 (MNa⁺), 352 (MH⁺ minus t-Bu).

### Synthesis of tert-butyl 2-(3-hydroxyphenyl)-7-azaspiro[3.5]nonane-7-carboxylate

A mixture of tert-butyl 2-[3-(benzyloxy)phenyl]-7-azaspiro[3.5]nonane-7-carboxylate (12.9 g, 31.7 mmol) and 10% Pd/C (2.00 g) in methanol (100 mL) and ethyl acetate (100 mL) was slurried under hydrogen at 45 psi overnight. The mixture was filtered through a pad of celite. The filtrate was concentrated and purified by flash chromatography (30% EtOAc/heptane) to give the title compound as a white solid (9.52 g). m/z 340 (MNa⁺), 262 (MH⁺ minus t-Bu).

### Synthesis of tert-butyl 2-(3-{[5-(trifluoromethyl)pyridin-2-yl]oxy}phenyl)-7-azaspiro[3.5]nonane-7-carboxylate

A mixture of 2-chloro-5-(trifluoromethyl)pyridine (579 mg, 3.19 mmol, 1.4 equiv; CAS #52334-81-3), tert-butyl 2-(3-hydroxyphenyl)-7-azaspiro[3.5]nonane-7-carboxylate (723 mg, 2.28 mmol, 1.0 equiv), and cesium carbonate (1.48 g, 4.56 mmol, 2.0 equiv) in DMF (7.0 mL) was stirred at 90° C. for 1 h. The reaction mixture was cooled to room temp and partitioned between ethyl acetate and water. The organic layer was washed with brine, dried over sodium sulfate, filtered, and concentrated to give the crude product as an oil which was purified by flash chromatography (0 to 20% ethyl acetate/heptanes) to yield the title compound as a clear viscous oil (900 mg, 85%). ¹H NMR (400 MHz, DMSO-d₆) δ ppm 8.55-8.58 (m, 1H), 8.22 (dd, J=9.0, 2.3 Hz, 1H), 7.37 (t, J=7.8 Hz, 1H), 7.21 (d, J=8.6 Hz, 1H), 7.15 (d, J=7.8 Hz, 1H), 7.04-7.07 (m, 1H), 7.00 (dd, J=7.4, 2.3 Hz, 1H), 3.48-3.60 (m, 1H), 3.30-3.35 (m, 2H), 3.17-3.22 (m, 2H), 2.20-2.28 (m, 2H), 1.79-1.87 (m, 2H), 1.60-1.65 (m, 2H), 1.42-1.47 (m, 2H), 1.39 (s, 9H). m/z 485 (MNa⁺).

### Synthesis of 2-(3-{[5-(trifluoromethyl)pyridin-2-yl]oxy}phenyl)-7-azaspiro[3.5]nonane hydrochloride

4 N HCl in dioxane (5 mL, 20 mmol) was added to a solution of tert-butyl 2-(3-{[5-(trifluoromethyl)pyridin-2-yl]oxy}phenyl)-7-azaspiro[3.5]nonane-7-carboxylate (888 mg, 1.92 mmol) in methylene chloride (15 mL) at room temp. After 1 h, the reaction mixture was concentrated in vacuo and dried under vacuum to give the title compound as a white solid (703 mg, 92%). ¹H NMR (400 MHz, DMSO-d₆) δ ppm 8.57 (d, J=2.7 Hz, 1H), 8.57 (br, s., 2H), 8.23 (dd, J=8.8, 2.5 Hz, 1H), 7.38 (t, J=7.8 Hz, 1H), 7.22 (d, J=8.6 Hz, 1H), 7.15 (d, J=7.8 Hz, 1H), 7.06-7.09 (m, 1H), 7.02 (dd, J=7.4, 2.3 Hz, 1H), 3.48-3.59 (m, 1H), 3.02-3.08 (m, 2H), 2.89-2.95 (m, 2H), 2.24-2.32 (m, 2H), 1.84-1.93 (m, 4H), 1.67-1.72 (m, 2H). m/z 363 (MH⁺).

### Synthesis of Phenyl pyridazin-3-ylcarbamate

To a solution of 3-amino-6-chloropyridazine (19.2 g, 148 mmol; CAS #5469-69-2) in EtOH (500 mL) was added 10% Pd catalyst on 1940 carbon (unreduced, 55% water). Triethylamine (50 mL) was added and the mixture was hydrogenated under 500 psi/mole for 1.9 h. The reaction was filtered and the ethanol was washed with aqueous NH₄Cl. The organic layer was concentrated to give pyridazin-3-amine as a white solid (11 g, 78% yield). MS (APCI 10V) AP+1 96.2. To a suspension of pyridazin-3-amine (5 g, 50 mmol) in THF (50 mL) and CH₃CN (70 mL) was added pyridine (5.10 mL, 63.1 mmol) followed by phenyl chloroformate (6.95 mL, 55.2 mmol) slowly. The reaction was stirred overnight. The reaction was filtered to remove the precipitate. The filtrate was concentrated and then taken up in CH₂Cl₂ which was washed with water. The organic layer was dried using SPE phase separators and concentrated. The residue was purified by silica gel column chromatography (0-5% MeOH/CH₂Cl₂). An undesired side product eluted first followed by the title compound which was concentrated to give a white solid (7.5 g, 70% yield). MS (APCI 10V) AP+1 216.12; ¹H NMR (400 MHz, DMSO-d₆) δ ppm 7.20-7.24 (m, 2H) 7.25-7.28 (m, 1H) 7.39-7.44 (m, 2H) 7.64-7.69 (m, 1H) 8.05 (dd, 1H) 8.94 (dd, 1H) 11.34 (s, 1H).

### Example 1

### Synthesis of N-pyridazin-3-yl-2-(3-{[5-(trifluoromethyl)pyridin-2-yl]oxy}phenyl)-7-azaspiro[3.5]nonane-7-carboxamide

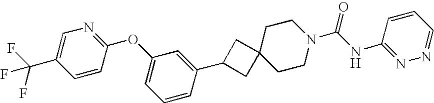

2-(3-{[5-(Trifluoromethyl)pyridin-2-yl]oxy}phenyl)-7-azaspiro[3.5]nonane hydrochloride (200 mg, 0.501 mmol, 1.0 equiv) was suspended in acetonitrile (4 mL) and treated with phenyl pyridazin-3-ylcarbamate (129 mg, 0.601 mmol, 1.2 equiv) and DIEA (0.349 mL, 2.00 mmol, 4.0 equiv). The reaction mixture was stirred at room temp for 1.5 h. The reaction mixture was concentrated and purified by reverse phase HPLC (10-95% acetonitrile/water/0.05% TFA). The fractions were isolated, concentrated, redissolved in acetonitrile, and filtered through a StratoSpheres™ PL-HCO₃ MP SPE tube (Polymer Laboratories, Amherst, Mass.) to neutralize any TFA. The filtrate was concentrated to give the title compound as a white solid (221 mg, 91%). ¹H NMR (400 MHz, DMSO-d₆) δ ppm 9.79 (s, 1H), 8.82 (dd, J=4.5, 1.4 Hz, 1H), 8.58 (d, J=2.3 Hz, 1H), 8.23 (dd, J=8.8, 2.5 Hz, 1H), 7.98 (dd, J=9.0, 1.6 Hz, 1H), 7.55 (dd, J=9.0, 4.7 Hz, 1H), 7.38 (t, J=7.8 Hz, 1H), 7.22 (d, J=8.6 Hz, 1H), 7.17 (d, J=7.8 Hz, 1H), 7.08 (t, J=2.0 Hz, 1H), 7.01 (dd, J=7.4, 2.0 Hz, 1H), 3.53-3.62 (m, 1H), 3.49-3.54 (m, 2H), 3.36-3.42 (m, 2H), 2.23-2.32 (m, 2H), 1.83-1.91 (m, 2H), 1.68-1.74 (m, 2H), 1.50-1.56 (m, 2H). m/z 484 (MH⁺).

### Synthesis of phenyl(3,4-dimethylisoxazol-5-yl)carbamate

Method A. 5-amino-3,4-dimethylisoxazole (Aldrich, 5.0 g, 40 mmol; CAS #19947-75-2) was dissolved in acetonitrile (75 mL) and cooled to 0° C. Phenyl chloroformate (5.91 mL, 46.8 mmol) dissolved in acetonitrile (50 mL) was then added slowly followed immediately by 1,8-bis(dimethylamino)naphthalene (Proton Sponge®, Aldrich; 9.56 g, 44.6 mmol) in acetonitrile (25 mL). The reaction was warmed to room temperature and stirred for 48 hours. The reaction was quenched with water (100 mL) and extracted with ethyl acetate (2×250 mL). The organics were dried with magnesium sulfate and concentrated to give a crude yellow oil. The crude product was purified by flash chromatography (ethyl acetate/heptane) to give the title compound as a white solid (9.02 g, 38.84 mmol, 90%). ¹H NMR (400 MHz, DMSO-d₆) δ ppm 10.70 (br. s., 1H), 7.40-7.47 (m, 2H), 7.26-7.30 (m, 1H), 7.21-7.25 (m, 2H), 2.16 (s, 3H), 1.86 (s, 3H). m/z 233 (MH⁺).

Method B. A three necked 5 L RB flask equipped with nitrogen bubbler and thermo pocket, was purged well with nitrogen for 20 min at room temp. Phenyl chloroformate (120.1 mL, 0.93 mol) in acetonitrile (1 L) was added to the stirred solution of 5-amino-3,4-dimethylisoxazole (AKSCIENTIFIC; 100 g, 0.89 mol) in acetonitrile (1.5 L) at <10° C. over 38 min under nitrogen followed by addition of 1,8-bis(dimethylamino)naphthalene (Proton Sponge®, Aldrich; 189.9 g, 0.886 mol) portionwise over 27 min. After stirring at <10° C. for 10 min, resulting reaction mixture was stirred at room temperature for 112 h under nitrogen atmosphere. After completion of the reaction (monitored by TLC, 30% EtOAc/hexane), the solid was filtered off and washed with EtOAc (2×375 mL). The filtrate was diluted with water (1.25 L) and EtOAc (2.5 L) and shook well. The layers were separated and the aqueous layer was back extracted with EtOAc (1.25 L). The organic layers were dried over sodium sulfate and concentrated under reduced pressure at 28° C. to afford residue as a greenish oil. The residue was dissolved in EtOAc (2.5 L), washed with water (3×600 mL), dried over sodium sulfate, and concentrated under reduced pressure at 28° C. to afford compound the title compound (207 g) as greenish yellow solid, which was dissolved in EtOAc (1 L), stirred with charcoal (20.7 g) for 30 min at room temperature and filtered through celite, washing the celite with EtOAc. Upon concentration of filtrate under reduced pressure at 28° C. the title compound was obtained as white solid which was dissolved in EtOAc (1080 mL) and heptane (1080 mL) and stirred for 10 min at room temperature. Crystallization initiated upon stirring. To this was added heptane (2220 mL) over a period of 30 min. The suspension was stirred for 30 min at room temperature. The solid was filtered and washed with heptane (2×150 mL) to afford the first crop of the title compound as a white crystalline solid (105 g). The mother liquor was concentrated under reduced pressure at 28° C. to afford 100 g crude product, which was recrystallized from EtOAc/heptane using the above crystallization method to obtain another 48 g of the title compound as second crop. The total yield was 153 g (74%). ¹H NMR (acetone-d₆, 400 MHz) δ ppm 9.5 (1H, bs), 7.44-7.40 (2H, m), 7.26 (1H, d, J=7.04 Hz), 7.22 (1H, d, J=8.64 Hz), 2.18 (3H, s), 1.92 (3H, s).

### Example 2

### Synthesis of N-(3,4-dimethylisoxazol-5-yl)-2-(3-{[5-(trifluoromethyl)pyridin-2-yl]oxy}phenyl)-7-azaspiro[3.5]nonane-7-carboxamide

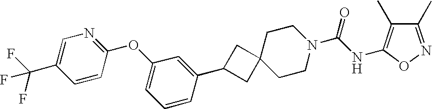

The title compound was prepared from 2-(3-{[5-(Trifluoromethyl)pyridin-2-yl]oxy}phenyl)-7-azaspiro[3.5]nonane hydrochloride (118.5 mg) and phenyl(3,4-dimethylisoxazol-5-yl)carbamate (82.7 mg) as described for Example 1. The reaction mixture was concentrated and purified by reverse phase HPLC (10-95% acetonitrile/water/0.05% TFA) to give the title compound as a white solid (107 mg, 72%). ¹H NMR (400 MHz, DMSO-d₆) δ ppm 9.09 (s, 1H), 8.58 (s, 1H), 8.23 (dd, J=8.8, 2.2 Hz, 1H), 7.39 (t, J=8.1 Hz, 1H), 7.22 (d, J=8.8 Hz, 1H), 7.17 (d, J=8.1 Hz, 1H), 7.08 (s, 1H), 7.02 (dd, J=8.1, 2.2 Hz, 1H), 3.51-3.63 (m, 1H), 3.41-3.47 (m, 2H), 3.29-3.34 (m, 2H), 2.24-2.31 (m, 2H), 2.12 (s, 3H), 1.83-1.92 (m, 2H), 1.73 (s, 3H), 1.66-1.72 (m, 2H), 1.49-1.54 (m, 2H). m/z 501 (MH⁺).

### Synthesis of phenyl 1,2-benzisoxazol-3-ylcarbamate

A solution of 1,2-benzisoxazol-3-amine (1.00 g; CAS #36216-80-5) and triethylamine (1.09 mL) in acetonitrile (5 mL) was added dropwise to at 0° C. solution of phenyl chloroformate (0.989 mL) in THF (20 mL). The reaction was stirred at 0° C. for 1 h and then allowed to warm to room temp overnight. The reaction was diluted with ethyl acetate and washed with 1N HCl and brine. The organic layer was dried over sodium sulfate, filtered, and concentrated to give the crude product as a reddish brown solid. The solid was triturated with refluxing diisopropyl ether, cooled to room temp, and filtered to give the final product as a tan solid (1.22 g, 64%). m/z 255 (MH⁺).

### Example 3

### Synthesis of N-1,2-benzisoxazol-3-yl-2-(3-{[5-(trifluoromethyl)pyridin-2-yl]oxy}phenyl)-7-azaspiro[3.5]nonane-7-carboxamide

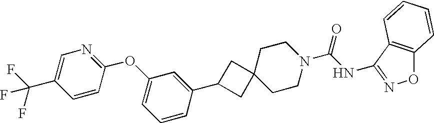

The title compound was prepared from 2-(3-{[5-(Trifluoromethyl)pyridin-2-yl]oxy}phenyl)-7-azaspiro[3.5]nonane hydrochloride (118.5 mg) and phenyl 1,2-benzisoxazol-3-ylcarbamate (90.5 mg) as described for Example 1. The reaction mixture was concentrated and purified by reverse phase HPLC (10-95% acetonitrile/water/0.05% TFA) to give the title compound as a white solid (143 mg, 92%). ¹H NMR (400 MHz, DMSO-d₆) δ ppm 9.85 (s, 1H), 8.59 (s, 1H), 8.24 (dd, J=8.8, 2.9 Hz, 1H), 7.80 (d, J=8.1 Hz, 1H), 7.56-7.67 (m, 2H), 7.39 (t, J=7.7 Hz, 1H), 7.31 (t, J=6.6 Hz, 1H), 7.23 (d, J=8.8 Hz, 1H), 7.18 (d, J=8.1 Hz, 1H), 7.10 (s, 1H), 7.02 (d, J=8.1 Hz, 1H), 3.56-3.66 (m, 1H), 3.51-3.57 (m, 2H), 3.39-3.45 (m, 2H), 2.26-2.35 (m, 2H), 1.86-1.93 (m, 2H), 1.74 (d, J=5.1 Hz, 2H), 1.57 (d, J=5.9 Hz, 2H). m/z 523 (MH⁺).

### Synthesis of tert-butyl 2-hydroxy-2-[3-(trifluoromethoxy)phenyl]-7-azaspiro[3.5]nonane-7-carboxylate

3-(Trifluoromethoxy)bromobenzene (32.2 g, 134 mmol; CAS #2252-44-0) was added to a solution of isopropylmagnesium chloride lithium chloride complex in THF (1.3 M solution, 101 mL, 132 mmol; Aldrich) at −5° C. under nitrogen. The solution was allowed to slowly warm to rt overnight, and the Grignard solution was added to a solution of tert-butyl 2-oxo-7-azaspiro[3.5]nonane-7-carboxylate (16.0 g, 66.9 mmol) in THF (300 mL) at 0° C. via cannula. After 1.5 h, the reaction was quenched with saturated ammonium chloride and extracted with ethyl acetate. The organic layers were dried over sodium sulfate, filtered, and concentrated to give the crude alcohol as an off-white solid (27.75 g, quant.). m/z 346 (MH⁺ minus t-Bu), 424 (MNa⁺).

### Synthesis of 2-[3-(trifluoromethoxy)phenyl]-7-azaspiro[3.5]nonane hydrochloride

A solution of crude tert-butyl 2-hydroxy-2-[3-(trifluoromethoxy)phenyl]-7-azaspiro[3.5]nonane-7-carboxylate (27.75 g, 66.9 mmol) and triethylsilane (45 mL, 280 mmol) in methylene chloride (350 mL) was treated with borontrifluoride diethyl etherate (16.5 mL, 134 mmol) and trifluoroacetic acid (25 mL, 340 mmol) at −15° C. After 1.5 h, the reaction was quenched with saturated sodium bicarbonate and extracted with ethyl acetate. The organic layers were washed with brine, dried over magnesium sulfate, filtered, and concentrated. The crude amine was dissolved in diethyl ether/dioxane and treated with 4N HCl/dioxane (20 mL). The precipitate was filtered and washed with diethyl ether to give the title compound as a white solid (11.3 g, 52.5%). m/z 286 (MH⁺).

### Example 4

### Synthesis of N-(3,4-dimethylisoxazol-5-yl)-2-[3-(trifluoromethoxy)phenyl]-7-azaspiro[3.5]nonane-7-carboxamide

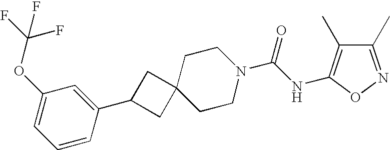

Method A. The title compound was prepared from 2-[3-(trifluoromethoxy)phenyl]-7-azaspiro[3.5]nonane hydrochloride (4.00 g) and phenyl(3,4-dimethylisoxazol-5-yl)carbamate (3.46 g) as described for Example 12, below. The crude compound was purified by flash chromatography (40 to 60% ethyl acetate/heptane) and then recrystallized from ethyl acetate/heptane to give the title compound as a white solid (3.27 g, 62%). ¹H NMR (400 MHz, DMSO-d₆) δ ppm 9.10 (s, 1H), 7.45 (t, J=8.1 Hz, 1H), 7.31 (d, J=8.1 Hz, 1H), 7.15-7.22 (m, 2H), 3.54-3.67 (m, 1H), 3.42-3.48 (m, 2H), 3.29-3.34 (m, 2H), 2.25-2.34 (m, 2H), 2.12 (s, 3H), 1.83-1.91 (m, 2H), 1.74 (s, 3H), 1.68-1.72 (m, 2H), 1.50-1.55 (m, 2H). m/z 424 (MH⁺).

Method B. A suspension of nickel iodide (30.9 mg, 0.099 mmol, 0.06 equiv.; Strem), trans-2-aminocyclohexanol (15.0 mg, 0.099 mmol, 0.06 equiv.; Alfa-Aesar), 3-trifluoromethoxyphenyl boronic acid (677 mg, 3.29 mmol, 2.0 equiv), and NaHMDS (634 mg, 3.29 mmol, 2 equiv.) in anhydrous 2-propanol (3.3 mL) was sparged with argon for 5 min. 2-Bromo-7-aza-spiro[3.5]nonane-7-carboxylic acid tert-butyl ester (500 mg, 1.64 mmol, 1.0 equiv) was added, and the reaction mixture was warmed to 70° C. for 12 h. Another 300 mg of trifluoromethoxyphenyl boronic acid and LHMDS were added and the reaction was stirred at 70° C. for another 4 h. The mixture was cooled to room temp and filtered through a plug of silica gel with 50% ethyl acetate/hexane (120 mL) and then evaporated to give an oil. The oil was purified by flash chromatography (0-15% ethyl acetate/heptane) to give a white solid, 540 mg. The white solid was dissolved in methylene chloride (10 mL) and treated with 4 N HCl in dioxane (3 mL). After stirring for 1 h at room temp, the reaction mixture was concentrated to dryness. A mixture of the amine hydrochloride salt and the carbamate in acetonitrile (5 mL) was treated with DIEA (1.14 mL, 6.57 mmol) and stirred 1.75 h at it and then was concentrated under nitrogen overnight. The residue was dissolved in DMF/MeOH and purified by reverse phase HPLC (5 to 95% acetonitrile/water/0.05% TFA; 25 min gradient) to give the title compound as a white solid (89.5 mg, 13%).

### Synthesis of 2-Hydroxy-7-aza-spiro[3.5]nonane-7-carboxylic acid tert-butyl ester

Sodium borohydride (15.16 g, 0.4 mol) was added in portionwise to a stirred solution of tert-butyl 2-oxo-7-azaspiro[3.5]nonane-7-carboxylate (80 g, 0.33 mol) in methanol (800 mL) at 0° C., and the resulting reaction mixture was stirred for 1 hour at 0° C. After completion of the reaction (monitored by TLC in 50% ethyl acetate in hexane, rf 0.4, iodine active), the methanol was evaporated under reduced pressure and residue was diluted with brine and extracted with ethyl acetate. The organic layer was dried over sodium sulfate and evaporated. The crude material obtained was triturated with hexane the title compound as a white solid (70 g, 86%). ¹H NMR (400 MHz, DMSO-d₆) δ ppm 4.90 (d, J=6.3 Hz, 1H), 4.05-4.13 (m, 1H), 3.15-3.27 (m, 4H), 2.08-2.15 (m, 2H), 1.50-1.58 (m, 2H), 1.38-1.41 (m, 4H), 1.38 (s, 9H). m/z 186 (MH⁺ minus t-Bu).

### Synthesis of 2-Bromo-7-aza-spiro[3.5]nonane-7-carboxylic acid tert-butyl ester

A solution of 2-hydroxy-7-aza-spiro[3.5]nonane-7-carboxylic acid tert-butyl ester (4.75 g, 19.7 mmol) in THF at 0° C. was treated with triphenylphosphine (10.3 g, 39.4 mmol) followed by carbon tetrabromide (13.1 g, 39.4 mmol). After 1 h, the reaction mixture was warmed to room temp. After 2 h, the reaction mixture was diluted with diethyl ether, filtered, and concentrated. The crude product was purified by flash chromatography (10% ethyl acetate/heptanes) to give the title compound as a white solid (3.05 g, 51%). ¹H NMR (400 MHz, DMSO-d₆) δ ppm 4.59-4.70 (m, 1H), 3.11-3.22 (m, 4H), 2.50-2.58 (m, 2H), 2.11-2.19 (m, 2H), 1.47-1.53 (m, 2H), 1.41-1.47 (m, 2H), 1.33 (s, 9H). m/z 248, 250 (MH⁺ minus t-Bu).

### Synthesis of tert-butyl 2-(3-methoxyphenyl)-7-azaspiro[3.5]nonane-7-carboxylate

A solution of the 3-methoxyphenylmagnesium bromide (1.0 M in THF, 4.93 mL, 4.93 mmol, 3.00 equiv; Aldrich) was added dropwise over 1.2 h with a syringe pump to a stirred mixture of 2-bromo-7-aza-spiro[3.5]nonane-7-carboxylic acid tert-butyl ester (500 mg, 1.64 mmol, 1 equiv)), Fe(acac)₃ (29.3 mg, 0.082 mmol, 0.05 equiv; CAS #14024-18-1), TMEDA (0.025 mL, 0.164 mmol, 0.10 equiv, CAS #110-18-9), and HMTA (11.6 mg, 0.082 mmol, 0.05 equiv; CAS #100-97-0) in THF (10 mL) at 0 C. After the completion of the addition, the reaction mixture was stirred for 45 min at 0 C, and then quenched with satd ammonium chloride. The aqueous phase was extracted with ethyl acetate. The organic phase was dried over sodium sulfate, filtered, and concentrated. The crude oil was purified by flash chromatography (10 to 25% ethyl acetate/heptane) to give the title compound as a clear oil which solidified (445 mg, 81.7%). m/z 276 (MH⁺ minus t-Bu).

### Example 5

### Synthesis of N-(3,4-dimethylisoxazol-5-yl)-2-(3-methoxyphenyl)-7-azaspiro[3.5]nonane-7-carboxamide

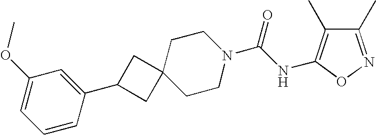

A solution of tert-butyl 2-(3-methoxyphenyl)-7-azaspiro[3.5]nonane-7-carboxylate (547 mg, 1.65 mmol, 1 equiv) in dichloromethane (10 mL) was treated with 4 N HCl in dioxane (3 mL, 12 mmol) at room temperature. After 1.25 h, the mixture was concentrated to dryness to give the crude amine hydrochloride. A mixture of the crude amine hydrochloride and phenyl(3,4-dimethylisoxazol-5-yl)carbamate (460 mg, 1.98 mmol, 1.2 equiv) in acetonitrile (5 mL) was treated with DIEA (1.15 mL, 6.60 mmol, 4 equiv) and stirred at room temp for 2 h and then concentrated. The residue was dissolved in DMF/methanol and purified by reverse phase HPLC (5 to 95% acetonitrile/water/0.05% TFA). The pure fractions were concentrated. A white precipitate formed and was filtered, washed with water and dried under vaccum overnight to give the title compound as a white solid (358 mg, 58.7%). ¹H NMR (400 MHz, DMSO-d₆) δ ppm 9.08 (s, 1H), 7.21 (t, J=8.1 Hz, 1H), 6.82 (d, J=8.1 Hz, 1H), 6.71-6.78 (m, 2H), 3.74 (s, 3H), 3.45-3.56 (m, 1H), 3.41-3.46 (m, 2H), 3.28-3.34 (m, 2H), 2.21-2.29 (m, 2H), 2.12 (s, 3H), 1.81-1.88 (m, 2H), 1.73 (s, 3H), 1.65-1.71 (m, 2H), 1.47-1.53 (m, 2H). m/z 370 (MH⁺).

### Synthesis of 2-[3-(trifluoromethyl)phenyl]-7-azaspiro[3.5]nonane hydrochloride

1-Bromo-3-(trifluoromethyl)benzene (11.2 g, 49.3 mmol, 3.00 equiv; CAS #401-78-5) was added to a solution of isopropylmagnesium chloride lithium chloride complex in THF (1.3 M solution, 39.2 mL, 50.9 mmol, 3.10 equiv; Aldrich) at 0° C. under nitrogen. The solution was allowed to slowly warm to room temperature overnight and then added dropwise over 4.6 h with a syringe pump to a stirred mixture of 2-bromo-7-aza-spiro[3.5]nonane-7-carboxylic acid tert-butyl ester (5.00 g, 16.4 mmol, 1 equiv)), Fe(acac)₃ (290 mg, 0.822 mmol, 0.05 equiv), TMEDA (0.247 mL, 1.64 mmol, 0.10 equiv), and HMTA (115 mg, 0.822 mmol, 0.05 equiv) in THF (100 mL) at 0 C and allowed to warm to room temperature overnight. LCMS showed reaction to be ˜80% complete, therefore a fresh solution of the Grignard reagent was prepared as outlined above from 20 mL of 1.3 M isopropylmagnesium chloride lithium chloride complex in THF and 1-bromo-3-(trifluoromethyl)benzene (3.5 mL, 1.5 equiv) at 0° C. The solution was allowed to warm to room temp for 4 h and then was added to the 0° C. reaction mixture over 2 h via syringe pump. After 45 min at 0 C, the reaction was quenched with satd ammonium chloride and was extracted with ethyl acetate. The organic phase was dried over sodium sulfate, filtered, and concentrated to give the Boc protected amine as an oil. The residue was dissolved in methylene chloride (50 mL) and treated with 4 N HCl/dioxane (20 mL). After 1.5 h, the reaction mixture was diluted with diethyl ether and filtered. The white ppt was washed with diethyl ether to give the title compound as a white solid (4.03 g, 80.2%). m/z 270 (MH⁺).

### Example 6

### Synthesis of N-(3,4-dimethylisoxazol-5-yl)-2-[3-(trifluoromethyl)phenyl]-7-azaspiro[3.5]nonane-7-carboxamide

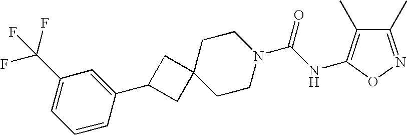

The title compound was prepared from 2-[3-(trifluoromethyl)phenyl]-7-azaspiro[3.5]nonane hydrochloride (336.8 mg) and phenyl(3,4-dimethylisoxazol-5-yl)carbamate (307 mg) as described for Example 12. The crude reaction mixture was concentrated, dissolved in DMF/methanol and purified by reverse phase HPLC (5 to 95% acetonitrile/water/0.05% TFA) to give the title compound as a white solid (247 mg, 55%). ¹H NMR (400 MHz, DMSO-d₆) δ ppm 9.10 (s, 1H), 7.51-7.63 (m, 4H), 3.59-3.71 (m, 1H), 3.43-3.48 (m, 2H), 3.29-3.34 (m, 2H), 2.28-2.35 (m, 2H), 2.12 (s, 3H), 1.86-1.94 (m, 2H), 1.74 (s, 3H), 1.68-1.73 (m, 2H), 1.50-1.56 (m, 2H). m/z 408 (MH⁺).

### Synthesis of 2-(3-methylphenyl)-7-azaspiro[3.5]nonane hydrochloride

A solution of m-tolylmagnesium chloride (1.0 M solution in THF, 73 mL, 4.5 equiv; Aldrich) added dropwise over 6 h with a syringe pump to a stirred mixture of 2-bromo-7-aza-spiro[3.5]nonane-7-carboxylic acid tert-butyl ester (5.00 g, 16.4 mmol, 1 equiv)), Fe(acac)₃ (290 mg, 0.822 mmol, 0.05 equiv), TMEDA (0.247 mL, 1.64 mmol, 0.10 equiv), and HMTA (115 mg, 0.822 mmol, 0.05 equiv) in THF (100 mL) at 0 C, and the reaction was allowed to warm to room temperature overnight. The reaction was quenched with satd ammonium chloride and extracted with ethyl acetate. The organic phase was dried over sodium sulfate, filtered, and concentrated to give the Boc protected amine as an oil. The residue was dissolved in methylene chloride (45 mL) and treated with 4 N HCl/dioxane (20 mL). After 1.5 h, the reaction mixture was diluted with diethyl ether and filtered. The white ppt was washed with diethyl ether to give the title compound as a white solid (2.91 g, 70%). m/z 216 (MH⁺).

### Example 7

### Synthesis of N-(3,4-dimethylisoxazol-5-yl)-2-(3-methylphenyl)-7-azaspiro[3.5]nonane-7-carboxamide

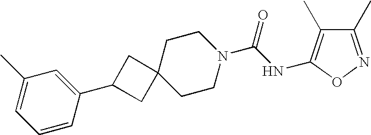

The title compound was prepared from 2-(3-methylphenyl)-7-azaspiro[3.5]nonane hydrochloride (300 mg) and phenyl(3,4-dimethylisoxazol-5-yl)carbamate (332 mg) as described for Example 12. The crude reaction mixture was concentrated, dissolved in DMF/methanol and purified by reverse phase HPLC (5 to 95% acetonitrile/water/0.05% TFA) to give the title compound as a white solid (279 mg, 66%). ¹H NMR (400 MHz, DMSO-d₆) δ ppm 9.09 (s, 1H), 7.18 (t, J=7.3 Hz, 1H), 6.96-7.07 (m, 3H), 3.45-3.54 (m, 1H), 3.41-3.47 (m, 2H), 3.29-3.34 (m, 2H), 2.29 (s, 3H), 2.22-2.29 (m, 2H), 2.12 (s, 3H), 1.80-1.88 (m, 2H), 1.74 (s, 3H), 1.66-1.73 (m, 2H), 1.48-1.54 (m, 2H). m/z 354 (MH⁺).

### Synthesis of tert-butyl 2-(3,4-dimethylphenyl)-7-azaspiro[3.5]nonane-7-carboxylate

The title compound was prepared from 3,4-dimethylphenylmagnesium chloride (0.5M solution in THF, 6.6 mL, 3.3 mmol, 2.0 equiv; Aldrich) and 2-bromo-7-aza-spiro[3.5]nonane-7-carboxylic acid tert-butyl ester (500 mg, 1.64 mmol, 1 equiv) as described for tert-butyl 2-(3-methoxyphenyl)-7-azaspiro[3.5]nonane-7-carboxylate. The crude oil was purified by flash chromatography (10 to 30% ethyl acetate/heptane) to give the title compound as a clear oil which solidified on standing (220 mg, 41%). m/z 274 (MH⁺ minus t-Bu).

### Example 8

### Synthesis of N-(3,4-dimethylisoxazol-5-yl)-2-(3,4-dimethylphenyl)-7-azaspiro[3.5]nonane-7-carboxamide

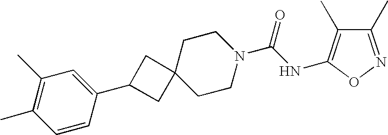

The title compound was prepared from tert-butyl 2-(3,4-dimethylphenyl)-7-azaspiro[3.5]nonane-7-carboxylate (222 mg) and phenyl(3,4-dimethylisoxazol-5-yl)carbamate (188 mg) as described for Example 5. The crude reaction mixture was concentrated, dissolved in DMF/methanol and purified by reverse phase HPLC (5 to 95% acetonitrile/water/0.05% TFA) to give the title compound as a white solid (143 mg, 58%). ¹H NMR (400 MHz, DMSO-d₆) δ ppm 9.09 (s, 1H), 7.05 (d, J=7.3 Hz, 1H), 7.01 (s, 1H), 6.95 (d, J=7.3 Hz, 1H), 3.41-3.51 (m, 3H), 3.29-3.34 (m, 2H), 2.21-2.28 (m, 2H), 2.20 (s, 3H), 2.17 (s, 3H), 2.12 (s, 3H), 1.78-1.86 (m, 2H), 1.74 (s, 3H), 1.65-1.71 (m, 2H), 1.48-1.53 (m, 2H). m/z 368 (MH⁺).

### Example 9

### Synthesis of 2-{3-[(5-bromopyrimidin-2-yl)oxy]phenyl}-N-pyridazin-3-yl-7-azaspiro[3.5]nonane-7-carboxamide

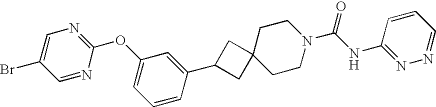

Step 1. A mixture of 5-bromo-2-chloropyrimidine (311 mg, 1.61 mmol, 1.4 equiv), tert-butyl 2-(3-hydroxyphenyl)-7-azaspiro[3.5]nonane-7-carboxylate (365 mg, 1.15 mmol, 1.0 equiv), and cesium carbonate (749 mg, 2.3 mmol, 2.0 equiv) in DMF (3.5 mL) was stirred at 90° C. for 1 h. The reaction mixture was cooled to room temp and partitioned between ethyl acetate and water. The organic layer was washed with satd sodium bicarbonate and brine, dried over sodium sulfate, filtered, and concentrated to give the crude biaryl ether. Step 2. The residue was dissolved in methylene chloride (10 mL) and treated with 4 N HCl in dioxane (3 mL). After stirring for 3 h at room temp, the reaction mixture was concentrated to dryness to give the crude amine hydrochloride salt. Step 3. A mixture of the amine hydrochloride salt and phenyl pyridazin-3-ylcarbamate (297 mg, 1.38 mmol, 1.2 equiv) in acetonitrile (5 mL) was treated with DIEA (0.801 mL, 4.60 mmol, 4 equiv) and stirred 1 h at room temp and then was concentrated. The residue was dissolved in DMF/MeOH and purified by reverse phase HPLC (5 to 95% acetonitrile/water/0.05% TFA). The pure fractions were concentrated and partitioned between ethyl acetate and satd bicarbonate solution. The organic phase was dried over sodium sulfate, filtered, and concentrated. The oil was crystallized from ethyl acetate to give the title compound as a white solid (426 mg, 74%). ¹H NMR (400 MHz, DMSO-d₆) δ ppm 9.81 (s, 1H), 8.79-8.85 (m, 3H), 7.98 (d, J=8.1 Hz, 1H), 7.56 (dd, J=8.8, 4.4 Hz, 1H), 7.37 (t, J=7.7 Hz, 1H), 7.16 (d, J=7.3 Hz, 1H), 7.11 (s, 1H), 7.03 (d, J=7.3 Hz, 1H), 3.53-3.61 (m, 1H), 3.49-3.55 (m, 2H), 3.36-3.42 (m, 2H), 2.23-2.31 (m, 2H), 1.83-1.91 (m, 2H), 1.68-1.73 (m, 2H), 1.50-1.56 (m, 2H). m/z 495, 497 (MH⁺).

### Example 10

### Synthesis of 2-{3-[(5-bromopyridin-2-yl)oxy]phenyl}-N-pyridazin-3-yl-7-azaspiro[3.5]nonane-7-carboxamide

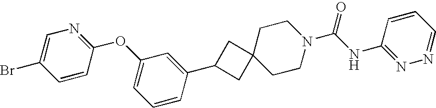

The title compound was prepared from tert-butyl 2-(3-hydroxyphenyl)-7-azaspiro[3.5]nonane-7-carboxylate (365 mg), 5-bromo-2-chloropyridine (310 mg; CAS #53939-30-3) and phenyl pyridazin-3-ylcarbamate (297 mg) as described for Example 9 (Step 1 was stirred at 90° C. overnight rather than 1 h) to give the title compound as an off-white solid (354 mg, 62%). ¹H NMR (400 MHz, DMSO-d₆) δ ppm 9.81 (s, 1H), 8.83 (d, J=5.9 Hz, 1H), 8.28 (s, 1H), 8.06 (dd, J=8.8, 2.9 Hz, 1H), 7.98 (d, J=8.1 Hz, 1H), 7.56 (dd, J=8.8, 4.4 Hz, 1H), 7.35 (t, J=7.7 Hz, 1H), 7.13 (d, J=8.1 Hz, 1H), 7.05 (s, 1H), 7.00-7.04 (m, 1H), 6.95 (dd, J=8.1, 2.2 Hz, 1H), 3.49-3.61 (m, 3H), 3.37-3.42 (m, 2H), 2.23-2.31 (m, 2H), 1.82-1.90 (m, 2H), 1.68-1.73 (m, 2H), 1.50-1.55 (m, 2H). m/z 494, 496 (MH⁺).

### Example 11

### Synthesis of 2-{3-[(5-bromopyridin-2-yl)oxy]phenyl}-N-(3,4-dimethylisoxazol-5-yl)-7-azaspiro[3.5]nonane-7-carboxamide

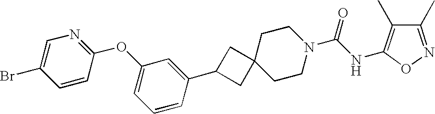

A solution of 5-bromo-2-chloropyridine (0.5 mmol) in dioxane (2 mL) was treated with tert-butyl 2-(3-hydroxyphenyl)-7-azaspiro[3.5]nonane-7-carboxylate (80 mg, 0.25 mmol), DMA (0.25 mL), and NaHMDS (0.6 N in toluene; 0.5 mL, 0.300 mmol). The mixture was heated under microwave irradiation at 185° C. for 1 h in a Biotage Initiator 60. Upon completion of the reaction, the solvent was evaporated in vacuo. The residue was reconstituted in dichloroethane (2 mL) and washed with water (2×1 mL). The organic layer was passed through Celite. The filtrate was concentrated. The resulting residue was dissolved in 20% trifluoroacetic acid/dichloromethane and shaken at room temp for 2 h. The volatiles were removed in vacuo to provide the crude amine as a TFA salt. The residue was dissolved in DMSO (1 mL). 0.5 mL of this solution (˜0.125 mmol) was combined with a 0.5 M solution of phenyl(3,4-dimethylisoxazol-5-yl)carbamate in DMSO (0.25 mL, 0.125 mmol) and N-methyl morpholine (0.100 mL). The reaction was shaken at 60° C. for 2 h. The reaction mixture was purified by reverse phase HPLC (acetonitrile/water/0.05% TFA) to give the title compound (13.55 mg). LCMS (Phenomenex Gemini C18 4.6×50 mm 5 μm (0.04% Formic Acid, 0.01% TFA/MeCN)) t_{R} 2.32 min; m/z 511.45 (MH⁺).

### Synthesis of 2-(4-Fluoro-3-methylphenyl)-7-azaspiro[3.5]nonane hydrochloride

To a solution of 5-bromo-2-fluorotoluene (2.50 mL, 20.1 mmol, 2.40 equiv) in THF (30 mL) at −78° C. was added a solution of sec-butyllithium (1.4 M in cyclohexane, 14.6 mL, 20.5 mmol, 2.45 equiv; Aldrich) dropwise. The mixture was warmed to −40° C. for 45 min, and then was transferred by cannula to a 0° C. solution of tert-butyl 2-oxo-7-azaspiro[3.5]nonane-7-carboxylate (2.00 g, 8.36 mmol) in THF (40 mL). The solution was allowed to warm to room temp slowly overnight. The reaction was quenched with saturated ammonium chloride and extracted with ethyl acetate. The organic layers were dried over sodium sulfate, filtered, and concentrated to give the crude alcohol as a yellow oily solid. A solution of the crude alcohol and triethylsilane (6.68 mL, 41.8 mmol, 5 equiv) in methylene chloride (40 mL) was treated with borontrifluoride diethyl etherate (2.06 mL, 16.7 mmol, 2 equiv) and trifluoroacetic acid (3.10 mL, 41.8 mmol, 5 equiv) at 0° C. After 1 h at 0° C., the reaction was quenched with saturated sodium bicarbonate and extracted with diethyl ether. The organic layers were extracted with 3 N HCl. The aqueous layer was washed with diethyl ether and then neutralized with 2.5 N NaOH. The milky suspension was extracted with diethyl ether. The ether layers were dried over magnesium sulfate, filtered, and treated with 4N HCl/dioxane (3 mL). The mixture was filtered to give the title compound as a white solid (740 mg, 33%). m/z 234 (MH⁺).

### Example 12

### Synthesis of N-(3,4-dimethylisoxazol-5-yl)-2-(4-fluoro-3-methylphenyl)-7-azaspiro[3.5]nonane-7-carboxamide

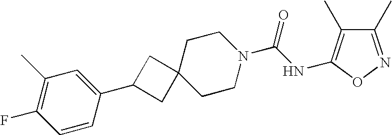

A mixture of 2-(4-fluoro-3-methylphenyl)-7-azaspiro[3.5]nonane hydrochloride (297 mg, 1.10 mmol, 1 equiv) and phenyl(3,4-dimethylisoxazol-5-yl)carbamate (307 mg, 1.32 mmol, 1.2 equiv) in acetonitrile (4 mL) was treated with diisopropylethylamine (0.766 mL, 4.40 mmol, 4.0 equiv) and stirred for 1.5 h at room temp. The reaction was partitioned between ethyl acetate and saturated ammonium chloride. The organic layer was dried over sodium sulfate, filtered, and concentrated. The crude compound was purified by flash chromatography (20 to 80% ethyl acetate/heptane) and then recrystallized from ethyl acetate to give the title compound as a white solid (203 mg, 50%). ¹H NMR (400 MHz, DMSO-d₆) δ ppm 9.04 (s, 1H), 7.09-7.14 (m, 1H), 6.97-7.06 (m, 2H), 3.41-3.50 (m, 1H), 3.37-3.42 (m, 2H), 3.25-3.29 (m, 2H), 2.19-2.25 (m, 2H), 2.18 (d, J=2.0 Hz, 3H), 2.08 (s, 3H), 1.75-1.83 (m, 2H), 1.70 (s, 3H), 1.62-1.67 (m, 2H), 1.45-1.50 (m, 2H). m/z 372 (MH⁺).

### Synthesis of tert-butyl 2-(3-chlorophenyl)-2-hydroxy-7-azaspiro[3.5]nonane-7-carboxylate

To a solution of tert-butyl 2-oxo-7-azaspiro[3.5]nonane-7-carboxylate (2.00 g, 8.36 mmol, 1 equiv) in THF (40 mL) at 0° C. was added 3-chlorophenylmagnesium bromide (0.5 M in THF, 33.4 mL, 16.7 mmol, 2.0 equiv; Aldrich). After 3 h, the reaction was quenched with saturated ammonium chloride and extracted with ethyl acetate. The organic layers were dried over sodium sulfate, filtered, and concentrated to give the title compound as a viscous oil (3.22 g).

### Synthesis of 2-(3-chlorophenyl)-7-azaspiro[3.5]nonane

A solution of the crude tert-butyl 2-(3-chlorophenyl)-2-hydroxy-7-azaspiro[3.5]nonane-7-carboxylate (2.94 g, 8.36 mmol) and triethylsilane (6.7 mL, 41.8 mmol) in methylene chloride (40 mL) was treated with borontrifluoride diethyl etherate (2.06 mL, 16.7 mmol) and trifluoroacetic acid (3.10 mL, 41.8 mmol) at 0° C. After 1 h at 0° C., the reaction was concentrated and purified by reverse phase HPLC. The pure fractions were concentrated and partitioned between ethyl acetate and saturated sodium bicarbonate. The organic layer was filtered and concentrated to give the title compound as a white solid (1.23 g, 62%).

### Example 13

### Synthesis of 2-(3-chlorophenyl)-N-(3,4-dimethylisoxazol-5-yl)-7-azaspiro[3.5]nonane-7-carboxamide

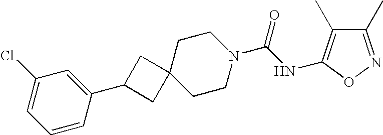

The title compound was prepared from 2-(3-chlorophenyl)-7-azaspiro[3.5]nonane (300 mg) and phenyl(3,4-dimethylisoxazol-5-yl)carbamate (354 mg) as described for Example 12. The crude reaction mixture was concentrated, dissolved in DMF/methanol and purified by reverse phase HPLC (5 to 95% acetonitrile/water/0.05% TFA). The pure fractions were concentrated to near dryness and then partitioned between ethyl acetate and satd sodium bicarbonate. The organic layer was dried over sodium sulfate, filtered, and concentrated to give a waxy white solid which was recrystallized from ethyl acetate/heptane to give the title compound as a white solid (139 mg, 29%). ¹H NMR (400 MHz, DMSO-d₆) δ ppm 9.04 (s, 1H), 7.34 (t, J=7.7 Hz, 1H), 7.29 (s, 1H), 7.20-7.27 (m, 2H), 3.50-3.62 (m, 1H), 3.42-3.48 (m, 2H), 3.30-3.36 (m, 2H), 2.25-2.32 (m, 2H), 2.13 (s, 3H), 1.83-1.91 (m, 2H), 1.75 (s, 3H), 1.68-1.72 (m, 2H), 1.50-1.56 (m, 2H). m/z 374 (MH⁺).

### Synthesis of tert-butyl 2-(3-chloro-4-fluorophenyl)-2-hydroxy-7-azaspiro[3.5]nonane-7-carboxylate

Isopropylmagnesium chloride solution in THF (2.0 M, 165 mL, 329 mmol; Aldrich) was added to solution of 1-bromo-3-chloro-4-fluorobenzene (70.0 g, 334 mmol) in THF (100 mL) in an ice/salt bath at a rate such that the temp ranged from −10 to −5° C. The solution was stirred in the ice/salt bath which was allowed to slowly warm to room temp overnight. To a solution of tert-butyl 2-oxo-7-azaspiro[3.5]nonane-7-carboxylate (40.0 g, 167 mmol) in THF (400 mL) at 0° C. was added the recooled (0° C.) Grignard solution via cannula portionwise such that the reaction temperature did not exceed 10° C. After 1.0 h, the reaction was carefully quenched at 0° C. with satd ammonium chloride (500 mL), diluted with water (100 mL) to dissolve solids, and extracted with ethyl acetate (500 mL). The aqueous layer was extracted a second time with ethyl acetate (200 mL). The organic layers were dried over sodium sulfate, filtered, and concentrated to give the crude alcohol as an off-white solid (61.1 g). The crude alcohol can be used as is or further purified by flash chromatography (30% ethyl acetate/heptane). m/z 314 (MH⁺ minus t-Bu).

### Synthesis of 2-(3-Chloro-4-fluorophenyl)-7-azaspiro[3.5]nonane hydrochloride

A −15° C. (ice/salt bath) solution of the crude tert-butyl 2-(3-chloro-4-fluorophenyl)-2-hydroxy-7-azaspiro[3.5]nonane-7-carboxylate (61.1 g, 165 mmol) and triethylsilane (110 mL, 700 mmol) in methylene chloride (350 mL) was treated via syringe with borontrifluoride diethyl etherate (42 mL, 340 mmol) followed by trifluoroacetic acid (reaction warmed to −2° C. during addition). The reaction was stirred at −10° C. for 45 min. The reaction was quenched with satd sodium bicarbonate (600 mL) dropwise until basic (removed bath during quench). The layers were separated and the aqueous layer extracted with dichloromethane (2×150 mL). The organic layers were washed with satd sodium bicarbonate, dried over magnesium sulfate, filtered, and concentrated to give the crude amine as an off-white solid (44.6 g). The crude amine was suspended in THF (300 mL) and treated with 100 mL of 2N HCl/diethyl ether. The solution was concentrated and diluted with diethyl ether. The resultant precipitate was filtered and washed with diethyl ether to give the title compound as a white solid (20.2 g, 42%). ¹H NMR (400 MHz, DMSO-d₆) δ ppm 8.77 (br. s., 2H), 7.41 (dd, J=7.2, 2.1 Hz, 1H), 7.27-7.33 (m, 1H), 7.19-7.24 (m, 1H), 3.41-3.53 (m, 1H), 2.96-3.04 (m, 2H), 2.83-2.91 (m, 2H), 2.19-2.27 (m, 2H), 1.80-1.88 (m, 4H), 1.65-1.71 (m, 2H). m/z 254 (MH⁺).

### Example 14

### Synthesis of 2-(3-chloro-4-fluorophenyl)-N-(3,4-dimethylisoxazol-5-yl)-7-azaspiro[3.5]nonane-7-carboxamide

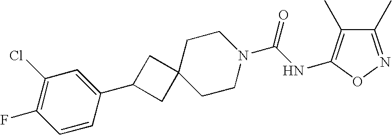

A mixture of 2-(3-chloro-4-fluorophenyl)-7-azaspiro[3.5]nonane hydrochloride (20.0 g, 68.9 mmol) and phenyl (3,4-dimethylisoxazol-5-yl)carbamate (19.4 g, 83.5 mmol, 1.2 equiv) in acetonitrile (300 mL) was treated with DIEA (48.0 mL, 276 mmol, 4 equiv) and stirred for 1.5 h at room temp. The brown solution was concentrated under reduced pressure. The resultant brown oil was partitioned between ethyl acetate and satd ammonium chloride. Not all of the solid dissolved, so the organic layer was filtered to give a portion of the title compound as a white solid (3.44 g). The filtrate was washed with satd ammonium chloride, water, satd sodium bicarbonate, and brine. The organic layer was dried over sodium sulfate, filtered, and concentrated to give a brown solid. The brown solid was suspended in ethyl acetate (50 mL), filtered, and washed with ethyl acetate (˜50 mL) to give another portion of the title compound as an off-white solid (12.62 g). The filtrate was concentrated to give an oil which was purified by flash chromatrography (30 to 70% ethyl acetate/heptane) to give a third portion of the title compound as a white solid (7.44 g) affording a total yield of 23.5 g, 87% yield. The three portions were combined and suspended in ˜100 mL boiling ethyl acetate. Approximately 50 mL heptane was added and the mixture was cooled to room temp overnight. The precipitate was filtered, washed with 1:1 ethyl acetate/heptane, and dried under vacuum to give the title compound as a white solid (21.57 g, 80%). ¹H NMR (400 MHz, DMSO-d₆) δ ppm 9.03 (br. s., 1H), 7.40 (dd, J=7.2, 2.1 Hz, 1H), 7.27-7.32 (m, 1H), 7.20-7.25 (m, 1H), 3.45-3.56 (m, 1H), 3.37-3.43 (m, 2H), 3.24-3.29 (m, 2H), 2.22 (td, J=9.2, 2.3 Hz, 2H), 2.08 (s, 3H), 1.77-1.85 (m, 2H), 1.70 (s, 3H), 1.62-1.67 (m, 2H), 1.46-1.51 (m, 2H). m/z 392 (MH⁺).

### Synthesis of tert-butyl 2-[(methylsulfonyl)oxy]-7-azaspiro[3.5]nonane-7-carboxylate

To the stirred solution of tert-butyl 2-hydroxy-7-azaspiro[3.5]nonane-7-carboxylate (70 g, 0.29 mol) in DCM (800 ml), was added methanesulfonyl chloride (24.7 ml, 0.32 mol) followed by triethylamine (60.6 ml, 0.45 mol) at 0° C., and the resulting reaction mixture was stirred for 45 minutes at the same temperature. After completion (monitored by TLC in 50% ethyl acetate in hexane, Rf=0.6, iodine active), reaction mixture was washed with saturated sodium bicarbonate solution and total organic layer was dried over sodium sulfate and evaporated under reduced pressure. Residue was triturated with hexane the title compound as a white solid (87 g, 94%). ¹H NMR (400 MHz, CDCl₃) δ ppm 5.03-4.99 (1H, t), 3.34-3.28 (4H, m), 2.97 (3H, s), 2.43-2.37 (2H, m), 2.09-2.04 (2H, m), 1.56-1.50 (4H, m), 1.43 (9H, s). m/z 320.2.

### Synthesis of tert-butyl 2-cyano-7-azaspiro[3.5]nonane-7-carboxylate

To the stirred solution of tert-butyl 2-[(methylsulfonyl)oxy]-7-azaspiro[3.5]nonane-7-carboxylate (62 g, 0.2 mol) in DMF (400 ml), potassium iodide (1.45 g, 0.02 mol) was added followed by addition of sodium cyanide (8.56 g, 0.35 mol) and the resulting reaction mixture was warmed to 120° C. and stirred for 72 hours. After completion (monitored by TLC in 50% ethyl acetate in hexane, Rf=0.7, iodine active), reaction mixture was cooled to room temperature and diluted with saturated solution of sodium bicarbonate (500 ml), and the organic layer was washed with water (3×250 ml) and dried over sodium sulfate. The organic layer was evaporated under reduced pressure and the crude obtained was purified by column chromatography using (100-200 mesh) silica gel in 15% ethyl acetate in hexane to afford the title compound as a white solid (32 g, 66%). ¹H NMR (400 MHz, CDCl₃) δ ppm 3.32-3.27 (4H, q), 3.07-3.03 (1H, m), 2.26-2.13 (4H, m), 1.63-1.58 (2H, m), 1.54-1.51 (2H, t), 1.4 (9H, s). GC-MS: 250.

### Synthesis of tert-butyl 2-[amino(hydroxyimino)methyl]-7-azaspiro[3.5]nonane-7-carboxylate

tert-Butyl 2-cyano-7-azaspiro[3.5]nonane-7-carboxylate (800 mg, 3.2 mmol) and hydroxylamine hydrochloride (333 mg, 4.8 mmol) were dissolved in ethanol (12 mL). To this solution was added triethylamine (0.67 mL, 4.8 mmol). The mixture was heated to 80° C. overnight. Additional hydroxylamine hydrochloride (333 mg, 4.8 mmol) was added followed by triethyamine (0.3 mL) and heating was continued overnight. The mixture was partially concentrated and then filtered. The solid was washed with cold EtOH. The filtrate was concentrated to dryness. The residue was washed with ethyl acetate and the ethyl acetate was decanted and concentrated. The residue was purified on silica gel column chromatograph (5% methanol/CH₂Cl₂) to give the title compound as a clear oil that solidified upon standing (320 mg, 35%). ¹H NMR (400 MHz, DMSO-d₆) δ ppm 8.83 (s, 1H), 5.25 (s, 2H), 3.22-3.30 (m, 2H), 3.12-3.21 (m, 2H), 2.78-2.92 (m, 1H), 1.89 (d, J=8.9 Hz, 4H), 1.46-1.55 (m, 2H), 1.34-1.45 (m, 11H). m/z 228 (MH⁺−tBu).

### Synthesis of tert-butyl 2-{5-[4-(trifluoromethoxy)phenyl]-1,2,4-oxadiazol-3-yl}-7-azaspiro[3.5]nonane-7-carboxylate

The title compound was prepared from tert-butyl 2-[amino(hydroxyimino)methyl]-7-azaspiro[3.5]nonane-7-carboxylate (320 mg, 1.13 mmol) and 4-(trifluoromethoxy)benzoyl chloride (330 mg, 1.47 mmol) as described for tert-butyl 2-{5-[4-(trifluoromethyl)phenyl]-1,2,4-oxadiazol-3-yl}-7-azaspiro[3.5]nonane-7-carboxylate. The crude compound was purified on silica gel (20% ethyl acetate/heptane) to give the title compound as a white solid (420 mg, 82%). ¹H NMR (400 MHz, CDCl₃) δ ppm 8.21 (d, J=8.9 Hz, 2H), 7.39 (d, J=8.9 Hz, 2H), 3.65-3.78 (m, 1H), 3.40-3.48 (m, 2H), 3.31-3.38 (m, 2H), 2.21-2.38 (m, 4H), 1.69-1.75 (m, 2H), 1.64-1.69 (m, 2H), 1.48 (s, 9H). m/z 398 (MH⁺−tBu).

### Synthesis of 2-{5-[4-(trifluoromethoxy)phenyl]-1,2,4-oxadiazol-3-yl}-7-azaspiro[3.5]nonane trifluoroacetate

The title compound was prepared from tert-butyl 2-{5-[4-(trifluoromethoxy)phenyl]-1,2,4-oxadiazol-3-yl}-7-azaspiro[3.5]nonane-7-carboxylate (420 mg, 0.92 mmol) in the same manner as described for 2-{5-[4-(trifluoromethyl)phenyl]-1,2,4-oxadiazol-3-yl}-7-azaspiro[3.5]nonane trifluoroacetate to give the title compound as the trifluoroacetate salt (690 mg). ¹H NMR (400 MHz, DMSO-d₆) δ ppm 8.15-8.20 (m, 2H), 7.57 (d, J=8.2 Hz, 2H), 3.63-3.75 (m, 1H), 3.01 (br. s., 2H), 2.88-2.97 (m, 2H), 2.23-2.32 (m, 2H), 2.06-2.15 (m, 2H), 1.78-1.85 (m, 2H), 1.66-1.73 (m, 2H). m/z 354 (MH⁺).

### Example 15

### Synthesis of N-(3,4-dimethylisoxazol-5-yl)-2-{5-[4-(trifluoromethoxy)phenyl]-1,2,4-oxadiazol-3-yl}-7-azaspiro[3.5]nonane-7-carboxamide

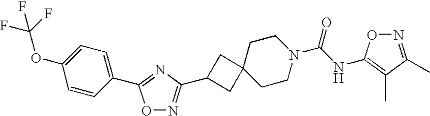

The title compound was prepared from 2-{5-[4-(trifluoromethoxy)phenyl]-1,2,4-oxadiazol-3-yl}-7-azaspiro[3.5]nonane trifluoroacetate (400 mg, 0.53 mmol) and phenyl(3,4-dimethylisoxazol-5-yl)carbamate (124 mg, 0.53 mmol) in the same manner as described for Example 33. The crude compound was purified on silica gel (50% ethyl acetate/heptane) to give the title compound as a white solid (210 mg, 80%). ¹H NMR (400 MHz, CDCl₃) δ ppm 8.44 (s, 1H), 8.35 (d, J=7.9 Hz, 1H), 7.88 (d, J=8.2 Hz, 1H), 7.71 (t, J=7.9 Hz, 1H), 6.64 (br. s., 1H), 3.70-3.82 (m, 1H), 3.48-3.55 (m, 2H), 3.39-3.46 (m, 2H), 2.28-2.43 (m, 4H), 2.21 (s, 3H), 1.90 (s, 3H), 1.81-1.87 (m, 2H), 1.76-1.81 (m, 2H). m/z 492 (MH⁺).

### Synthesis of 4-nitrophenyl 2-(3-methylphenyl)-7-azaspiro[3.5]nonane-7-carboxylate

A mixture of 2-(3-methylphenyl)-7-azaspiro[3.5]nonane hydrochloride (2.60 g, 10.3 mmol, 1.00 equiv), dioxane (100 mL), and said sodium bicarbonate (50 mL) was slurried at room temp. A solution of 4-nitrophenyl chloroformate (2.18 g, 10.8 mmol, 1.05 equiv) in dioxane (50 mL) was added slowly to the milky white mixture. The reaction was stirred at room temp for 2 h. The dioxane was partially removed in vacuo and the resulting aqueous suspension was extracted with ethyl acetate. The organic extracts were dried over sodium sulfate, filtered, and concentrated to give the crude product (5.43 g). The crude product was slurried in ethyl acetate (˜50 mL) for 1 h. The precipitate was filtered to give the product as a white solid (1.77 g). The mother liquor was placed in a freezer overnight to produce a second crop (1.04 g). The mother liquor was concentrated and purified by flash chromatography (5 to 25% ethyl acetate/heptanes) to give a third batch (730 mg). The three batches were combined to give the title compound as a white solid (3.54 g, 90.1%). ¹H NMR (400 MHz, DMSO-d₆) δ ppm 8.27 (d, J=9.0 Hz, 2H), 7.44 (d, J=9.0 Hz, 2H), 7.19 (t, J=7.4 Hz, 1H), 7.07 (s, 1H), 7.04 (d, J=7.4 Hz, 1H), 6.99 (d, J=7.4 Hz, 1H), 3.60-3.67 (m, 1H), 3.45-3.57 (m, 3H), 3.33-3.41 (m, 1H), 2.30 (s, 3H), 2.25-2.32 (m, 2H), 1.84-1.92 (m, 2H), 1.74-1.83 (m, 2H), 1.56-1.66 (m, 2H). m/z 381 (MH⁺).

### Example 16

### Synthesis of 2-(3-methylphenyl)-N-(1-methyl-1H-tetrazol-5-yl)-7-azaspiro[3.5]nonane-7-carboxamide

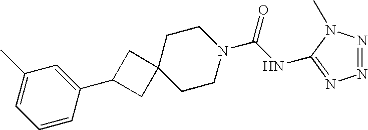

A 0.18 M stock solution of the 4-nitrophenyl 2-(3-methylphenyl)-7-azaspiro[3.5]nonane-7-carboxylate in anhydrous DMA was prepared. A 0.72 M stock suspension of NaH (60% suspension in mineral oil) in anhydrous DMA was prepared. To a vial containing 1-methyl-1H-tetrazol-5-amine (135 umol, 1.5 equiv; CAS #5422-44-6) was added an aliquot of the NaH in DMA stock suspension (0.250 mL, 0.180 mmol). The vial was capped and shaken for 10 min. To the vial was added an aliquot of the 4-nitrophenyl 2-(3-methylphenyl)-7-azaspiro[3.5]nonane-7-carboxylate stock solution (0.500 mL, 0.090 mmol, 1.0 equiv). The vial was capped and shaken at room temperature for 16 h followed by 65° C. for 4 h. The reaction was quenched with water (0.100 mL) and concentrated in vacuo. The crude residue was reconstituted in DMSO and purified by reverse phase HPLC (10 to 95% acetonitrile/water/0.05% TFA) to give the title compound (17.9 mg). ¹H NMR (400 MHz, DMSO-d₆) δ ppm 9.69 (br. s., 1H), 7.19 (t, J=7.7 Hz, 1H), 7.01-7.09 (m, 2H), 6.99 (d, J=6.6 Hz, 1H), 3.79 (s, 3H), 3.45-3.54 (m, 3H), 2.29 (s, 3H), 2.21-2.29 (m, 2H), 1.82-1.91 (m, 2H), 1.70-1.76 (m, 2H), 1.52-1.58 (m, 2H). LCMS (Phenomenex Gemini C18 4.6×50 mm 5 μm (0.04% Formic Acid, 0.01% TFA/MeCN)) t_{R}=1.9 min; m/z 341.45 (MH⁺).

### Example 17

### Synthesis of 2-(3-methylphenyl)-N-(6-phenyl-1,2,4,5-tetrazin-3-yl)-7-azaspiro[3.5]nonane-7-carboxamide

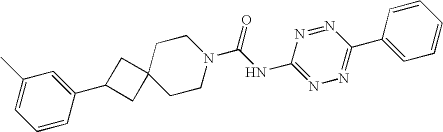

The title compound was prepared from 4-nitrophenyl 2-(3-methylphenyl)-7-azaspiro[3.5]nonane-7-carboxylate and 6-phenyl-1,2,4,5-tetrazin-3-amine (CAS #14418-30-5) as described for Example 16 (3.8 mg). LCMS (Phenomenex Gemini C18 4.6×50 mm 5 μm (0.04% Formic Acid, 0.01% TFA/MeCN)) t_{R}=2.23 min; m/z 415.35 (MH⁺).

### Synthesis of 2-(3-fluoro-5-methylphenyl)-7-azaspiro[3.5]nonane hydrochloride

To a solution of tert-butyl 2-oxo-7-azaspiro[3.5]nonane-7-carboxylate (3.00 g, 12.54 mmol) in THF (50 mL) at 0° C. was added 3-fluoro-5-methylphenylmagnesium bromide (prepared from stirring 1-bromo-3-fluoro-5-methylbenzene (4.74 g, 25.10 mmol) and isopropyl magnesium chloride (19.0 mL, 24.70 mmol) in THF (10 mL) at r.t. for 14 hrs). After 1 h, the reaction was quenched with saturated ammonium chloride and extracted with ethyl acetate. The organic layers were dried over sodium sulfate, filtered, and concentrated to give the crude alcohol as a pale yellow oil. A solution of the crude alcohol and triethylsilane (8.4 mL, 53.0 mmol) in methylene chloride (50 mL) was treated with borontrifluoride diethyl etherate (3.09 mL, 25.1 mmol) and trifluoroacetic acid (4.7 mL, 63.0 mmol) at 0° C. After 1 h at 0° C., the reaction was quenched with saturated sodium bicarbonate and extracted with dichloromethane. The organics were washed with brine, dried over magnesium sulfate, filtered, and concentrated. The oil was diluted with ether and treated with 4N HCl/dioxane (4 mL). The precipitate was filtered and dried to give the title compound as a white solid (2.50 g, 52%).

### Example 18

### Synthesis of N-(3,4-dimethylisoxazol-5-yl)-2-(3-fluoro-5-methylphenyl)-7-azaspiro[3.5]nonane-7-carboxamide

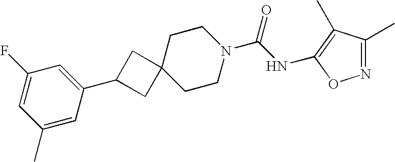

The title compound was prepared from 2-(3-fluoro-5-methylphenyl)-7-azaspiro[3.5]nonane hydrochloride (400 mg, 1.31 mmol) and phenyl(3,4-dimethylisoxazol-5-yl)carbamate (398 mg, 1.72 mmol) as described for Example 22. The crude compound was purified by reverse phase chromatography (acetonitrile/water), concentrated and then passed through a normal phase silica plug eluting with an ethyl acetate/5% methanol solution to give the title compound as a white solid (150 mg, 0.337 mmol, 31%). ¹H NMR (400 MHz, DMSO-d₆) δ ppm 9.02 (1H, s), 6.82 (2H, t, J=10.1 Hz), 3.45-3.53 (1H, m), 3.39-3.45 (2H, m), 3.28-3.33 (2H, m), 2.29 (3H, s), 2.24 (2H, dd, J=11.6, 9.1 Hz), 2.11 (3H, s), 1.79-1.90 (2H, m), 1.73 (3H, s), 1.65-1.70 (2H, m), 1.46-1.53 (2H, m) m/z 372.2 (MH⁺).

### Synthesis of 2-(2,3-difluorophenyl)-7-azaspiro[3.5]nonane hydrochloride

To a solution of tert-butyl 2-oxo-7-azaspiro[3.5]nonane-7-carboxylate (2.48 g, 10.36 mmol) in THF (50 mL) at 0° C. was added 2,3-difluorophenylmagnesium bromide (prepared from stirring 1-bromo-2,3-difluorobenzene (4.00 g, 20.73 mmol) and isopropyl magnesium chloride (15.7 mL, 20.4 mmol) in THF (10 mL) at r.t. for 14 hrs). After 1 h, the reaction was quenched with saturated ammonium chloride and extracted with ethyl acetate. The organic layers were dried over sodium sulfate, filtered, and concentrated to give the crude alcohol as a pale yellow oil. A solution of the crude alcohol and triethylsilane (7.0 mL, 44.0 mmol) in methylene chloride (50 mL) was treated with borontrifluoride diethyl etherate (2.56 mL, 20.7 mmol) and trifluoroacetic acid (3.9 mL, 52.0 mmol) at 0° C. After 1 h at 0° C., the reaction was quenched with saturated sodium bicarbonate and extracted with dichloromethane. The organics were washed with brine, dried over magnesium sulfate, filtered, and concentrated. The oil was diluted with ether and treated with 4N HCl/dioxane (4 mL). The precipitate was filtered and dried to give the title compound as a white solid (1.5 g, 61%).

### Example 19

### Synthesis of 2-(2,3-difluorophenyl)-N-(3,4-dimethylisoxazol-5-yl)-7-azaspiro[3.5]nonane-7-carboxamide

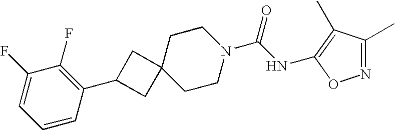

The title compound was prepared from 2-(2,3-difluorophenyl)-7-azaspiro[3.5]nonane hydrochloride (300 mg, 1.27 mmol) and phenyl(3,4-dimethylisoxazol-5-yl)carbamate (294 mg, 1.27 mmol) as described for Example 22. The crude compound was purified by reverse phase chromatography (acetonitrile/water), concentrated and then passed through a normal phase silica plug eluting with an ethyl acetate/5% methanol solution to give the title compound as a white solid (170 mg, 0.45 mmol, 35%). ¹H NMR (400 MHz, DMSO-d₆) δ ppm 9.02 (1H, s), 7.11-7.27 (3H, m), 3.73 (1H, t, J=9.1 Hz), 3.39-3.47 (2H, m), 3.28-3.34 (2H, m), 2.25-2.35 (2H, m), 2.11 (3H, s), 1.88-1.97 (2H, m), 1.68-1.75 (5H, m), 1.44-1.54 (2H, m) m/z 376.2(MH⁺).

### Synthesis of 2-(3,4-dichlorophenyl)-7-azaspiro[3.5]nonane hydrochloride

The title compound was prepared from 3,4-dichlorophenylmagnesium bromide (25 mL of 0.5 M solution in THF, 10 mmol; Aldrich) and tert-butyl 2-oxo-7-azaspiro[3.5]nonane-7-carboxylate (1.46 g, 6.10 mmol) as described for 2-[3-(trifluoromethoxy)phenyl]-7-azaspiro[3.5]nonane hydrochloride. The crude amine was dissolved in diethyl ether/methylene chloride and treated with 2N HCl/diethyl ether (5 mL). The mixture was concentrated and resuspended in hot methylene chloride/diethyl ether. The precipitate was filtered and washed with diethyl ether to give two crops of the title compound (814 mg, 44%). m/z 270 (MH⁺).

### Example 20

### Synthesis of 2-(3,4-dichlorophenyl)-N-(3,4-dimethylisoxazol-5-yl)-7-azaspiro[3.5]nonane-7-carboxamide

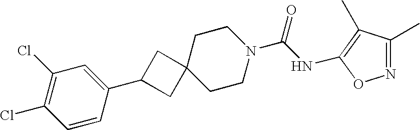

The title compound was prepared from 2-(3,4-dichlorophenyl)-7-azaspiro[3.5]nonane hydrochloride (407 mg) and phenyl(3,4-dimethylisoxazol-5-yl)carbamate (308 mg) as described for Example 12. The crude reaction mixture was concentrated, dissolved in DMF/methanol/TFA and purified by reverse phase HPLC (10 to 95% acetonitrile/water/0.05% TFA). The pure fractions were concentrated to near dryness and then partitioned between ethyl acetate and satd sodium bicarbonate. The organic layer was dried over sodium sulfate, filtered, concentrated, and recrystallized from ethyl acetate/heptane to give a 1:1 mixture of 2-(3,4-dichlorophenyl)-N-(3,4-dimethylisoxazol-5-yl)-7-azaspiro[3.5]non-1-ene-7-carboxamide and the title compound (70.6 mg, 13%). ¹H NMR (400 MHz, DMSO-d₆) δ ppm 9.09 (s, 1H), 7.48-7.50 (m, 1H), 7.37 (dd, J=8.1, 2.2 Hz, 1H), 7.27 (dd, J=8.4, 1.8 Hz, 1H), 3.51-3.61 (m, 1H), 3.42-3.47 (m, 2H), 3.30-3.35 (m, 2H), 2.24-2.32 (m, 2H), 2.14 (s, 3H), 1.83-1.91 (m, 2H), 1.77 (s, 3H), 1.67-1.72 (m, 2H), 1.50-1.56 (m, 2H). m/z 408 (MH⁺).

### Synthesis of 2-(5-chloro-2-fluorophenyl)-7-azaspiro[3.5]nonane hydrochloride

To a solution of tert-butyl 2-oxo-7-azaspiro[3.5]nonane-7-carboxylate (2.48 g, 10.36 mmol) in THF (50 mL) at 0° C. was added 4-chloro-1-fluorophenylmagnesium bromide (prepared from stirring 2-bromo-4-chloro-1-fluorobenzene (4.34 g, 20.73 mmol) and isopropyl magnesium chloride (15.7 mL, 20.4 mmol) in THF (10 mL) at r.t. for 14 hrs). After 1 h, the reaction was quenched with saturated ammonium chloride and extracted with ethyl acetate. The organic layers were dried over sodium sulfate, filtered, and concentrated to give the crude alcohol as a pale yellow oil. A solution of the crude alcohol and triethylsilane (7.0 mL, 44.0 mmol) in methylene chloride (50 mL) was treated with borontrifluoride diethyl etherate (2.56 mL, 20.7 mmol) and trifluoroacetic acid (3.9 mL, 52.0 mmol) at 0° C. After 1 h at 0° C., the reaction was quenched with saturated sodium bicarbonate and extracted with dichloromethane. The organics were washed with brine, dried over magnesium sulfate, filtered, and concentrated. The oil was diluted with ether and treated with 4N HCl/dioxane (4 mL). The precipitate was filtered and dried to give the title compound as a white solid (1.7 g, 64%).

### Example 21

### Synthesis of 2-(5-chloro-2-fluorophenyl)-N-(3,4-dimethylisoxazol-5-yl)-7-azaspiro[3.5]nonane-7-carboxamide

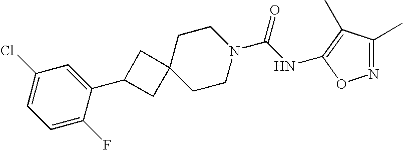

The title compound was prepared from 2-(5-chloro-2-fluorophenyl)-7-azaspiro[3.5]nonane hydrochloride (300 mg, 1.18 mmol) and phenyl(3,4-dimethylisoxazol-5-yl)carbamate (275 mg, 1.18 mmol) as described for Example 22. The crude compound was purified by reverse phase chromatography (acetonitrile/water), concentrated and then passed through a normal phase silica plug eluting with an ethyl acetate/5% methanol solution to give the title compound as a white solid (210 mg, 0.53 mmol, 45%). ¹H NMR (400 MHz, DMSO-d₆) δ ppm 9.02 (1H, s), 7.31-7.40 (1H, m), 7.23-7.31 (1H, m), 7.16 (1H, t, J=9.2 Hz), 3.63-3.70 (1H, m), 3.40-3.44 (2H, m), 2.21-2.31 (2H, m), 2.07-2.15 (3H, m), 1.87-1.97 (2H, m), 1.67-1.74 (5H, m), 1.47-1.53 (2H, m) m/z 392.2(MH⁺).

### Synthesis of 2-(3-ethylphenyl)-7-azaspiro[3.5]nonane hydrochloride

To a solution of tert-butyl 2-oxo-7-azaspiro[3.5]nonane-7-carboxylate (3.0 g, 12.54 mmol) in THF (50 mL) at 0° C. was added 3-ethylphenylmagnesium bromide (prepared from stirring 1-bromo-3-ethylbenzene (4.64 g, 25.1 mmol) and isopropyl magnesium chloride (19.0 mL, 24.7 mmol) in THF (10 mL) at r.t. for 14 hrs). After 1 h, the reaction was quenched with saturated ammonium chloride and extracted with ethyl acetate. The organic layers were dried over sodium sulfate, filtered, and concentrated to give the crude alcohol as a pale yellow oil. A solution of the crude alcohol and triethylsilane (8.4 mL, 53.0 mmol) in methylene chloride (50 mL) was treated with borontrifluoride diethyl etherate (3.09 mL, 25.1 mmol) and trifluoroacetic acid (4.7 mL, 63.0 mmol) at 0° C. After 1 h at 0° C., the reaction was quenched with saturated sodium bicarbonate and extracted with dichloromethane. The organics were washed with brine, dried over magnesium sulfate, filtered, and concentrated. The oil was diluted with ether and treated with 4N HCl/dioxane (4 mL). The precipitate was filtered and dried to give the title compound as a white solid (1.3 g, 45%).

### Example 22

### Synthesis of N-(3,4-dimethylisoxazol-5-yl)-2-(3-ethylphenyl)-7-azaspiro[3.5]nonane-7-carboxamide

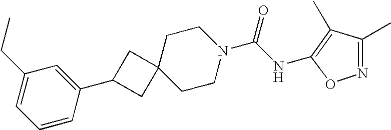

A mixture of 2-(3-ethylphenyl)-7-azaspiro[3.5]nonane hydrochloride (300 mg, 1.31 mmol, 1 equiv) and phenyl (3,4-dimethylisoxazol-5-yl)carbamate (304 mg, 1.31 mmol, 1 equiv) in acetonitrile (1 mL) was treated with diisopropylethylamine (0.740 mL, 5.24 mmol, 4.0 equiv) and stirred for 1 h at room temp. The reaction was concentrated and the residue was initially purified by reverse phase chromatography (acetonitrile/water) concentrated and then passed through a normal phase silica plug eluting with an ethyl acetate/5% methanol solution to give the title compound as a white solid (150 mg, 0.337 mmol, 31%). ¹H NMR (400 MHz, DMSO-d₆) δ ppm 8.97 (1H, s), 7.08-7.19 (1H, m), 6.78-7.05 (3H, m), 3.44 (1H, t, J=9.1 Hz), 3.36-3.41 (2H, m), 3.24-3.29 (2H, m), 2.53 (2H, q, J=7.7 Hz), 2.14-2.26 (2H, m), 2.06 (3H, s), 1.74-1.85 (2H, m), 1.68 (3H, s), 1.60-1.67 (2H, m), 1.41-1.49 (2H, m), 1.12 (3H, t, J=7.6 Hz) m/z 368.2 (MH⁺).

### Synthesis of 2-fluoro-2-[3-(trifluoromethoxy)phenyl]-7-azaspiro[3.5]nonane hydrochloride

A solution of tert-butyl 2-hydroxy-2-[3-(trifluoromethoxy)phenyl]-7-azaspiro[3.5]nonane-7-carboxylate (3.63 g, 9.04 mmol) in methylene chloride (60 mL) was treated with (diethylamino)sulfur trifluoride (DAST; 1.24 mL, 9.50 mmol, 1.05 equiv) at −78° C. After 2 h at −78° C., the reaction was quenched with water and diluted with methylene chloride. The organic layer was dried over magnesium sulfate, filtered, concentrated, and purified by flash chromatography (0 to 10% ethyl acetate/heptane) to give tert-butyl 2-fluoro-2-[3-(trifluoromethoxy)phenyl]-7-azaspiro[3.5]nonane-7-carboxylate (2.00 g, 4.96 mmol). m/z 348 (MH⁺ minus t-Bu). A solution of tert-butyl 2-fluoro-2-[3-(trifluoromethoxy)phenyl]-7-azaspiro[3.5]nonane-7-carboxylate (2.00 g, 4.96 mmol) in dichoromethane (30 mL) was treated with 4N HCl/dioxane (10 mL) at room temp. After 30 min, the reaction was concentrated to give the title compound as a white solid (1.70 g, quant.). ¹H NMR (400 MHz, DMSO-d₆) δ ppm 8.83 (br. s., 2H), 7.55 (d, J=7.8 Hz, 1H), 7.44-7.51 (m, 1H), 7.31-7.39 (m, 2H), 2.98-3.05 (m, 2H), 2.87-2.95 (m, 2H), 2.39-2.59 (m, 4H), 1.88-1.95 (m, 2H), 1.66-1.72 (m, 2H). m/z 304 (MH⁺).

### Example 23

### Synthesis of N-(3,4-dimethylisoxazol-5-yl)-2-fluoro-2-[3-(trifluoromethoxy)phenyl]-7-azaspiro[3.5]nonane-7-carboxamide

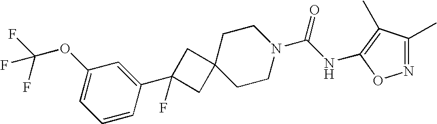

The title compound was prepared from 2-fluoro-2-[3-(trifluoromethoxy)phenyl]-7-azaspiro[3.5]nonane hydrochloride (500 mg) and phenyl(3,4-dimethylisoxazol-5-yl)carbamate (410 mg) as described for Example 13 to give the title compound as a white solid (420 mg, 65%). ¹H NMR (400 MHz, DMSO-d₆) δ ppm 9.06 (s, 1H), 7.57-7.63 (m, 1H), 7.51-7.56 (m, 1H), 7.36-7.43 (m, 2H), 3.43-3.48 (m, 2H), 3.33-3.38 (m, 2H), 2.42-2.60 (m, 4H), 2.13 (s, 3H), 1.76-1.80 (m, 2H), 1.75 (s, 3H), 1.52-1.57 (m, 2H). m/z 442 (MH⁺).

### Synthesis of 4-nitrophenyl 2-[3-(trifluoromethyl)phenyl]-7-azaspiro[3.5]nonane-7-carboxylate

The title compound was prepared from 2-[3-(trifluoromethyl)phenyl]-7-azaspiro[3.5]nonane hydrochloride (3.16 g) as described for 4-nitrophenyl 2-(3-methylphenyl)-7-azaspiro[3.5]nonane-7-carboxylate. The reaction suspension was partitioned between ethyl acetate and ½ satd sodium bicarbonate. The organic extract washed several times with satd sodium bicarbonate and brine, dried over sodium sulfate, filtered, concentrated and purified by flash chromatography (10 to 30% ethyl acetate/heptane) followed by purification by reverse phase HPLC (acetonitrile/water/0.05% TFA) to give the title compound as a white solid (3.00 g, 67%). m/z 435 (MH⁺).

### Example 24

### Synthesis of N-(1-methyl-1H-tetrazol-5-yl)-2-[3-(trifluoromethyl)phenyl]-7-azaspiro[3.5]nonane-7-carboxamide

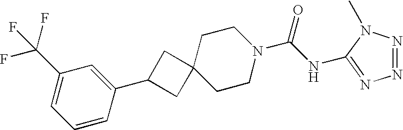

Sodium hydride (60% dispersion in mineral oil, 170 mg, 4.25 mmol, 2.05 equiv) was added portionwise to a solution of 1-methyl-1H-tetrazol-5-amine (411 mg, 4.14 mmol, 2.0 equiv; CAS #5422-44-6) in DMA (9 mL) and was stirred for 5 min at room temp. The mixture was treated with 4-nitrophenyl 2-[3-(trifluoromethyl)phenyl]-7-azaspiro[3.5]nonane-7-carboxylate (900 mg, 2.07 mmol, 1 equiv) and stirred overnight at room temp. The reaction was quenched with water, diluted with ethyl acetate and washed repeatedly with satd sodium bicarbonate. The organic layers were dried over sodium sulfate, filtered, concentrated, and purified by flash chromatography (40 to 80% ethyl acetate/heptanes). The product was recrystallized from ethyl acetate/heptane to give the title compound as a white solid (522 mg, 64%). ¹H NMR (400 MHz, DMSO-d₆) δ ppm 9.88 (s, 1H), 7.52-7.63 (m, 4H), 3.81 (s, 3H), 3.62-3.73 (m, 1H), 3.49-3.55 (m, 2H), 3.36-3.42 (m, 2H), 2.30-2.39 (m, 2H), 1.88-1.97 (m, 2H), 1.73-1.80 (m, 2H), 1.55-1.61 (m, 2H). m/z 395 (MH⁺).

### Example 25

### Synthesis of 2-{3-[2-(4-chlorophenoxy)ethoxy]phenyl}-N-(3,4-dimethylisoxazol-5-yl)-7-azaspiro[3.5]nonane-7-carboxamide

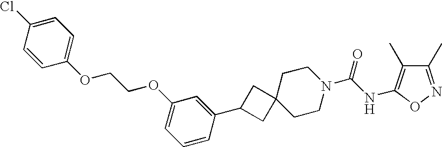

PS-PPh₃ (3 mmol/g loading factor, 1.15 g, 3.46 mmol, 2 equiv), 2-(4-chlorophenoxy)ethanol (326 mg, 1.89 mmol, 1.2 equiv), and tert-butyl 2-(3-hydroxyphenyl)-7-azaspiro[3.5]nonane-7-carboxylate (500 mg, 1.58 mmol, 1 equiv) were suspended in dichloromethane (40 mL). The mixture was shaken for 10 min and then treated with di-tert-butyl azodicarboxylate (DBAD; 725 mg, 3.15 mmol, 2 equiv). The mixture was shaken overnight. The polymer was filtered and washed with diethyl ether. The filtrate was concentrated and dissolved in dichloromethane (10 mL) and treated with TFA (3 mL). The mixture was stirred at room temperature for 0.5 h. The solvent and TFA were evaporated to dryness to furnish the amine trifluoroacetate salt, which was dissolved in acetonitrile (5 mL). 2.5 mL of this solution (˜0.79 mmol) was treated with phenyl (3,4-dimethylisoxazol-5-yl)carbamate (220 mg, 0.945 mmol) followed by diisopropylethylamine (1.00 mL, 5.74 mmol). The mixture was stirred at room temperature for 4 h. The reaction mixture was concentrated to dryness, dissolved in DMF/methanol, and purified by reverse phase HPLC (acetonitrile/water/0.05% TFA). The pure fractions were concentrated to give the title compound as an off-white solid (78 mg, 19%). ¹H NMR (400 MHz, DMSO-d₆) δ ppm 9.04 (s, 1H), 7.35 (d, J=9.5 Hz, 2H), 7.23 (t, J=7.7 Hz, 1H), 7.03 (d, J=8.8 Hz, 2H), 6.78-6.87 (m, 3H), 4.32 (s, 4H), 3.48-3.57 (m, 1H), 3.42-3.48 (m, 2H), 3.31-3.35 (m, 2H), 2.22-2.31 (m, 2H), 2.13 (s, 3H), 1.83-1.90 (m, 2H), 1.75 (s, 3H), 1.67-1.73 (m, 2H), 1.47-1.55 (m, 2H). m/z 510 (MH⁺).

### Example 26

### Synthesis of N-(3,4-dimethylisoxazol-5-yl)-2-[3-(2-phenoxyethoxy)phenyl]-7-azaspiro[3.5]nonane-7-carboxamide

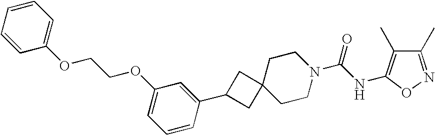

The title compound was prepared from 2-phenoxyethanol (261 mg), and tert-butyl 2-(3-hydroxyphenyl)-7-azaspiro[3.5]nonane-7-carboxylate (500 mg), and phenyl(3,4-dimethylisoxazol-5-yl)carbamate (220 mg) as described for Example 25. 73 mg, 20%. ¹H NMR (400 MHz, DMSO-d₆) δ ppm 9.04 (s, 1H), 7.32 (t, J=8.1 Hz, 2H), 7.23 (t, J=8.1 Hz, 1H), 6.94-7.02 (m, 3H), 6.78-6.87 (m, 3H), 4.32 (s, 4H), 3.48-3.58 (m, 1H), 3.42-3.48 (m, 2H), 3.32-3.36 (m, 2H), 2.23-2.31 (m, 2H), 2.13 (s, 3H), 1.82-1.92 (m, 2H), 1.75 (s, 3H), 1.67-1.73 (m, 2H), 1.50-1.55 (m, 2H). m/z 476 (MH⁺).

### Example 27

### Synthesis of 2-{3-[2-(2-chlorophenoxy)ethoxy]phenyl}-N-(3,4-dimethylisoxazol-5-yl)-7-azaspiro[3.5]nonane-7-carboxamide

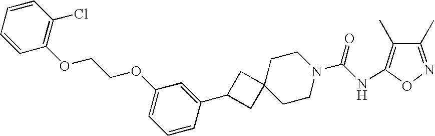

The title compound was prepared from 2-(2-chlorophenoxy)ethanol (326 mg), and tert-butyl 2-(3-hydroxyphenyl)-7-azaspiro[3.5]nonane-7-carboxylate (500 mg), and phenyl(3,4-dimethylisoxazol-5-yl)carbamate (220 mg) as described for Example 25. 95 mg, 24%. ¹H NMR (400 MHz, DMSO-d₆) δ ppm 9.04 (s, 1H), 7.44 (d, J=8.1 Hz, 1H), 7.30-7.36 (m, 1H), 7.20-7.26 (m, 2H), 6.96-7.02 (m, 1H), 6.80-6.87 (m, 3H), 4.38-4.44 (m, 2H), 4.32-4.39 (m, 2H), 3.48-3.57 (m, 1H), 3.42-3.48 (m, 2H), 3.31-3.36 (m, 2H), 2.22-2.32 (m, 2H), 2.13 (s, 3H), 1.82-1.91 (m, 2H), 1.75 (s, 3H), 1.67-1.73 (m, 2H), 1.48-1.55 (m, 2H). m/z 510 (MH⁺).

### Synthesis of 2-(2,2-difluoro-1,3-benzodioxol-4-yl)-7-azaspiro[3.5]nonane hydrochloride

To a solution of tert-butyl 2-oxo-7-azaspiro[3.5]nonane-7-carboxylate (1.5 g, 6.26 mmol) in THF (25 mL) at 0° C. was added 2,2-difluoro-1,3-benzodioxolephenylmagnesium bromide (prepared from stirring 4-bromo-2,2-difluoro-1,3-benzodioxole (2.97 g, 12.5 mmol) and isopropyl magnesium chloride (9.5 mL, 12.3 mmol) in THF (10 mL) at r.t. for 14 hrs). After 1 h, the reaction was quenched with saturated ammonium chloride and extracted with ethyl acetate. The organic layers were dried over sodium sulfate, filtered, and concentrated to give the crude alcohol as a pale yellow oil. A solution of the crude alcohol and triethylsilane (4.2 mL, 26.0 mmol) in methylene chloride (25 mL) was treated with borontrifluoride diethyl etherate (1.55 mL, 12.5 mmol) and trifluoroacetic acid (2.3 mL, 32.0 mmol) at 0° C. After 1 h at 0° C., the reaction was quenched with saturated sodium bicarbonate and extracted with dichloromethane. The organics were washed with brine, dried over magnesium sulfate, filtered, and concentrated. The oil was diluted with ether and treated with 4N HCl/dioxane (4 mL). The precipitate was filtered and dried to give the title compound as a white solid (0.86 g, 48%).

### Example 28

### Synthesis of 2-(2,2-difluoro-1,3-benzodioxol-4-yl)-N-(3,4-dimethylisoxazol-5-yl)-7-azaspiro[3.5]nonane-7-carboxamide

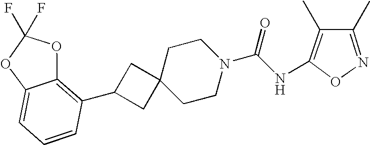

The title compound was prepared from 2-(2,2-difluoro-1,3-benzodioxol-4-yl)-7-azaspiro[3.5]nonane hydrochloride (200 mg, 0.71 mmol) and phenyl(3,4-dimethylisoxazol-5-yl)carbamate (165 mg, 0.71 mmol) as described for Example 22. The crude compound was purified by reverse phase chromatography (acetonitrile/water), concentrated and then passed through a normal phase silica plug eluting with an ethyl acetate/5% methanol solution to give the title compound as a white solid (200 mg, 0.48 mmol, 67%). ¹H NMR (400 MHz, DMSO-d₆) δ ppm 9.02 (1H, s), 7.09-7.24 (3H, m), 3.67 (1H, t, J=9.1 Hz), 3.40-3.47 (2H, m), 3.28-3.34 (2H, m), 2.24-2.35 (2H, m), 2.11 (3H, s), 1.93-2.02 (2H, m), 1.68-1.77 (5H, m), 1.48-1.54 (2H, m) m/z 420.2 (MH⁺).

### Synthesis of 2-(3-chloro-2-fluorophenyl)-7-azaspiro[3.5]nonane hydrochloride

The title compound was prepared from 3-chloro-2-fluorobromobenzene (2.81 g, 13.4 mmol) and tert-butyl 2-oxo-7-azaspiro[3.5]nonane-7-carboxylate (1.60 g, 6.70 mmol) as described for 2-[3-(trifluoromethoxy)phenyl]-7-azaspiro[3.5]nonane hydrochloride. The crude amine was dissolved in diethyl ether and treated with 2N HCl/diethyl ether (5 mL). The precipitate was filtered and washed with diethyl ether to give the title compound (1.47 g, 76%). m/z 254 (MH⁺).

### Example 29

### Synthesis of 2-(3-chloro-2-fluorophenyl)-N-(3,4-dimethylisoxazol-5-yl)-7-azaspiro[3.5]nonane-7-carboxamide

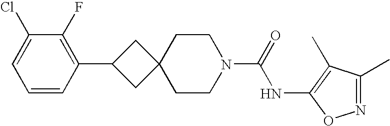

The title compound was prepared from 2-(3-chloro-2-fluorophenyl)-7-azaspiro[3.5]nonane hydrochloride (500 mg) and phenyl(3,4-dimethylisoxazol-5-yl)carbamate (480 mg) as described for Example 12. The crude product was purified by flash chromatography (30 to 60% ethyl acetate/heptane) and then recrystallized from ethyl acetate/heptane to give the title compound as a white solid (307 mg, 46%). ¹H NMR (400 MHz, DMSO-d₆) δ ppm 9.05 (s, 1H), 7.42 (t, J=7.3 Hz, 1H), 7.35 (t, J=7.3 Hz, 1H), 7.21 (t, J=7.7 Hz, 1H), 3.69-3.80 (m, 1H), 3.43-3.49 (m, 2H), 3.31-3.36 (m, 2H), 2.27-2.35 (m, 2H), 2.13 (s, 3H), 1.90-1.99 (m, 2H), 1.75 (s, 3H), 1.71-1.75 (m, 2H), 1.49-1.54 (m, 2H). m/z 392 (MH⁺).

### Synthesis of 2-[2-fluoro-3-(trifluoromethyl)phenyl]-7-azaspiro[3.5]nonane hydrochloride

The title compound was prepared from 2-fluoro-3-(trifluoromethyl)bromobenzene (2.81 g, 13.4 mmol) and tert-butyl 2-oxo-7-azaspiro[3.5]nonane-7-carboxylate (1.60 g, 6.70 mmol) as described for 2-[3-(trifluoromethoxy)phenyl]-7-azaspiro[3.5]nonane hydrochloride. The crude amine was dissolved in diethyl ether and treated with 2N HCl/diethyl ether (5 mL). The precipitate was filtered and washed with diethyl ether to give the title compound (1.42 g, 66%). m/z 288 (MH⁺).

### Example 30

### Synthesis of N-(3,4-dimethylisoxazol-5-yl)-2-[2-fluoro-3-(trifluoromethyl)phenyl]-7-azaspiro[3.5]nonane-7-carboxamide

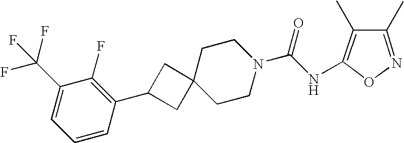

The title compound was prepared from 2-[2-fluoro-3-(trifluoromethyl)phenyl]-7-azaspiro[3.5]nonane hydrochloride (558 mg) and phenyl(3,4-dimethylisoxazol-5-yl)carbamate (480 mg) as described for Example 12. The crude product was purified by flash chromatography (30 to 60% ethyl acetate/heptane) and then recrystallized from ethyl acetate/heptane to give the title compound as a white solid (314 mg, 43%). ¹H NMR (400 MHz, DMSO-d₆) δ ppm 9.05 (s, 1H), 7.73 (t, J=7.3 Hz, 1H), 7.62 (t, J=7.0 Hz, 1H), 7.40 (t, J=7.7 Hz, 1H), 3.74-3.85 (m, 1H), 3.44-3.49 (m, 2H), 3.31-3.36 (m, 2H), 2.33 (t, J=10.3 Hz, 2H), 2.13 (s, 3H), 1.94-2.01 (m, 2H), 1.75 (s, 3H), 1.72-1.77 (m, 2H), 1.50-1.55 (m, 2H). m/z 426 (MH⁺).

### Synthesis of tert-butyl 2-(3-{[(trifluoromethyl)sulfonyl]oxy}phenyl)-7-azaspiro[3.5]nonane-7-carboxylate

In a 500 mL flask was added tert-butyl 2-(3-hydroxyphenyl)-7-azaspiro[3.5]nonane-7-carboxylate (2.0 g, 6.3 mmol), pyridine (1.50 mL, 18.9 mmol) in dichloromethane (65 mL). The mixture was cooled with an ice-bath while trifluoromethanesulfonic anhydride (1.60 mL, 9.45 mmol) was added dropwise. The mixture was stirred at room temperature for 1 hour. The mixture was washed with sodium bicarbonate and brine, and dried over sodium sulfate. The crude was purified by column chromatography (20% ethyl acetate/hexane) to give the title compound as a colorless oil (2.55 g, 90%). ¹H NMR (400 MHz, CDCl₃) δ ppm 7.38 (m, 1H), 7.22 (m, 1H), 7.08 (m, 2H), 3.66 (m, 1H), 3.42 (m, 2H), 3.32 (m, 2H), 2.35 (m, 2H), 1.90 (m, 2H), 1.72 (m, 2H), 1.45-1.55 (m, 2H), 1.45 (s, 9H). m/z 450 (MH⁺).

### Example 31

### Synthesis of N-(3,4-dimethylisoxazol-5-yl)-2-(3′-fluorobiphenyl-3-yl)-7-azaspiro[3.5]nonane-7-carboxamide

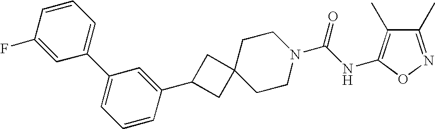

A 0.1 M solution of tert-butyl 2-(3-{[(trifluoromethyl)sulfonyl]oxy}phenyl)-7-azaspiro[3.5]nonane-7-carboxylate in DMF (1 mL), a 0.025 M solution of Pd(PPh₃)₄ in DMF (0.2 mL), and a 1 M aqueous solution of sodium carbonate (0.3 mL) were added to (3-fluorophenyl)boronic acid (0.125 mmol) in a vial (Note: the solutions were purged with nitrogen prior to addition). The vial was capped and heated to 100° C. for 18 h. The solvents were evaporated. The residue was portioned between dichloromethane (2 mL) and water (1 mL). The organic layer was treated with trifluoroacetic acid (0.5 mL). After 1 h at room temp, the solvents were evaporated. A 0.1 M solution of phenyl(3,4-dimethylisoxazol-5-yl)carbamate in acetonitrile (1.0 mL) and triethylamine (0.060 mmol) were added to the residue. The vial was capped and shook for 4 h at room temp. The solvents were evaporated and the residue was dissolved in DMSO (1.5 mL) and purified by reverse phase HPLC (acetonitrile/water/0.05% trifluoroacetic acid) to give the title compound (13.3 mg). LCMS (Phenomenex Gemini C18 4.6×50 mm 5 μm (0.04% Formic Acid, 0.01% TFA/MeCN)) t_{R}=2.22 min; m/z 434.35 (MH⁺).

### Synthesis of 2-(3-chlorophenyl)-2-fluoro-7-azaspiro[3.5]nonane hydrochloride

The title compound was prepared from tert-butyl 2-(3-chlorophenyl)-2-hydroxy-7-azaspiro[3.5]nonane-7-carboxylate (3.22 g) as described for 2-fluoro-2-[3-(trifluoromethoxy)phenyl]-7-azaspiro[3.5]nonane as a white solid (1.53 g, 63%). m/z 254 (MH⁺).

### Example 32

### Synthesis of 2-(3-chlorophenyl)-N-(3,4-dimethylisoxazol-5-yl)-2-fluoro-7-azaspiro[3.5]nonane-7-carboxamide

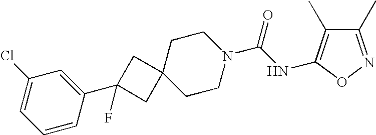

The title compound was prepared from 2-(3-chlorophenyl)-2-fluoro-7-azaspiro[3.5]nonane hydrochloride (500 mg) and phenyl(3,4-dimethylisoxazol-5-yl)carbamate (480 mg) as described for Example 12. The crude product was purified by flash chromatography (30 to 60% ethyl acetate/heptane) and then recrystallized from ethyl acetate/heptane to give the title compound as a white solid (433 mg, 64%). ¹H NMR (400 MHz, DMSO-d₆) δ ppm 9.06 (s, 1H), 7.42-7.52 (m, 4H), 3.42-3.48 (m, 2H), 3.32-3.38 (m, 2H), 2.40-2.59 (m, 4H), 2.13 (s, 3H), 1.76-1.80 (m, 2H), 1.75 (s, 3H), 1.50-1.56 (m, 2H). m/z 392 (MH⁺).

### Synthesis of tert-butyl 2-{5-[4-(trifluoromethyl)phenyl]-1,2,4-oxadiazol-3-yl}-7-azaspiro[3.5]nonane-7-carboxylate

To a solution of tert-butyl 2-[amino(hydroxyimino)methyl]-7-azaspiro[3.5]nonane-7-carboxylate (180 mg, 0.63 mmol) in THF was added DIEA (0.22 mL, 1.27 mmol) and p-(trifluoromethyl)benzoyl chloride (132 mg, 0.63 mmol). The mixture was heated to reflux for 12 hours. The reaction mixture was diluted with ethyl acetate and washed with brine (2×). The organic layer was dried (MgSO₄), filtered and concentrated. The residue was purified by silica gel chromatography (20% ethyl acetate/heptane) to give the title compound as a clear oil (240 mg, 86%). ¹H NMR (400 MHz, CDCl₃) δ ppm 8.43 (s, 1H), 8.34 (d, J=7.9 Hz, 1H), 7.87 (d, J=7.9 Hz, 1H), 7.71 (t, J=7.9 Hz, 1H), 3.64-3.81 (m, 1H), 3.39-3.50 (m, 2H), 3.28-3.40 (m, 2H), 2.20-2.40 (m, 4H), 1.70-1.77 (m, 2H), 1.64-1.70 (m, 2H), 1.48 (s, 9H). m/z 460 (MH⁺+Na).

### Synthesis of 2-{5-[4-(trifluoromethyl)phenyl]-1,2,4-oxadiazol-3-yl}-7-azaspiro[3.5]nonane trifluoroacetate

tert-Butyl 2-{5-[4-(trifluoromethyl)phenyl]-1,2,4-oxadiazol-3-yl}-7-azaspiro[3.5]nonane-7-carboxylate (180 mg, 0.41 mmol) was dissolved in 5 mL of dichloromethane. Trifluoroacetic acid (2 mL) was added and the mixture was stirred at room temperature for 2 hours. The mixture was concentrated and the residue was co-evaporated with methanol to give the title compound as a clear oil (300 mg). This material was used without further purification. ¹H NMR (400 MHz, methanol-d₆) δ ppm 8.42 (s, 1H), 8.40 (s, 1H), 8.00 (d, J=7.9 Hz, 1H), 7.85 (t, J=8.2 Hz, 1H), 3.75-3.89 (m, 1H), 3.19-3.27 (m, 2H), 3.10-3.19 (m, 2H), 2.40-2.51 (m, 2H), 2.30-2.40 (m, 2H), 2.00-2.10 (m, 2H), 1.90-2.00 (m, 2H). m/z 338 (MH⁺).

### Example 33

### Synthesis of N-(3,4-dimethylisoxazol-5-yl)-2-{5-[4-(trifluoromethyl)phenyl]-1,2,4-oxadiazol-3-yl}-7-azaspiro[3.5]nonane-7-carboxamide

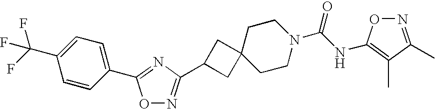

2-{5-[4-(Trifluoromethyl)phenyl]-1,2,4-oxadiazol-3-yl}-7-azaspiro[3.5]nonane (150 mg, 0.44 mmol) was dissolved in acetonitrile (3 mL). To this solution was added DIEA (0.31 mL, 1.78 mmol) followed by phenyl (3,4-dimethylisoxazol-5-yl)carbamate (114 mg, 0.49 mmol). The reaction mixture was stirred for 3 days at room temperature then concentrated and purified by silica gel column chromatography (30-60% ethyl acetate/heptane) to give the title compound as a white solid (95 mg, 45%). ¹H NMR (400 MHz, CDCl₃) δ ppm 8.44 (s, 1H), 8.35 (d, J=7.9 Hz, 1H), 7.88 (d, J=8.2 Hz, 1H), 7.71 (t, J=7.9 Hz, 1H), 6.64 (br. s., 1H), 3.48-3.55 (m, 2H), 3.39-3.46 (m, 2H), 2.28-2.43 (m, 4H), 2.21 (s, 3H), 1.90 (s, 3H), 1.81-1.87 (m, 2H), 1.76-1.81 (m, 2H). m/z 476 (MH⁺).

### Synthesis of phenyl(3-ethyl-4-methylisoxazol-5-yl)carbamate

To a solution of 5-amino-3-ethyl-4-methylisoxazole (5.63 g, 44.6 mmol, 1.0 equiv; CAS #153458-34-5) in acetonitrile (25 mL) at 0° C. was added triethylamine (6.53 mL, 46.8 mmol, 1.05 equiv) followed by phenyl chloroformate (5.91 mL, 46.8 mmol, 1.05 equiv) in 100 mL THF. After stirring at 0° C. for 1 h, the reaction was warmed to room temperature overnight. The reaction was diluted with ethyl acetate and washed with 2M HCl, water, saturated sodium bicarbonate, and brine. The organic layer was dried over magnesium sulfate, filtered, concentrated, and purified by flash chromatography (ethyl acetate/hexane) to give the title compound as a white solid (4.0 g). ¹H NMR (400 MHz, DMSO-d₆) δ ppm 10.71 (br. s., 1H), 7.41-7.45 (m, 2H), 7.26-7.30 (m, 1H), 7.22-7.26 (m, 2H), 2.58 (q, J=7.51 Hz, 2H), 1.87 (s, 3H), 1.17 (t, J=7.51 Hz, 3H). m/z 279.2 (MNa⁺).

### Example 34

### Synthesis of N-(3-ethyl-4-methylisoxazol-5-yl)-2-[3-(trifluoromethoxy)phenyl]-7-azaspiro[3.5]nonane-7-carboxamide

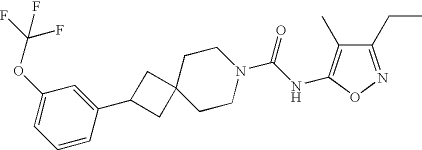

The title compound was prepared from 2-[3-(trifluoromethoxy)phenyl]-7-azaspiro[3.5]nonane hydrochloride (200 mg) and phenyl(3-ethyl-4-methylisoxazol-5-yl)carbamate (184 mg) as described for Example 12. The crude product was purified by flash chromatography (10 to 80% ethyl acetate/heptane) to give the title compound as a white solid (258 mg, 95%). ¹H NMR (400 MHz, DMSO-d₆) δ ppm 1.16 (t, J=7.50 Hz, 3H) 1.48-1.56 (m, 2H) 1.66-1.79 (m, 5H) 1.81-1.91 (m, 2H) 2.25-2.33 (m, 2H) 2.56 (s, 1H) 3.31-3.38 (m, 2H) 3.40-3.49 (m, 2H) 3.54-3.66 (m, 1H) 7.14-7.20 (m, 2H) 7.29 (d, J=7.69 Hz, 1H) 7.43 (t, J=7.87 Hz, 1H) 9.01 (s, 1H). m/z talc: 438.2004 found: 438.2181 (MH⁺).

### Synthesis of phenyl(5-methyl-1,3,4-oxadiazol-2-yl)carbamate

5-Methyl-1,3,4-oxadiazol-2-amine (2.37 g, 23.9 mmol; CAS #52838-39-8) was added to a 0° C. of phenyl chloroformate in THF (35.5 mL). After 1 h, the reaction was diluted with diethyl ether and filtered to give the title compound as a white solid (6.1 g, quant., ˜90% pure). ¹H NMR (400 MHz, DMSO-d₆) δ ppm 8.41 (br. s., 1H), 7.49 (t, J=8.0 Hz, 2H), 7.29-7.39 (m, 3H), 2.41 (s, 3H). m/z 220 (MH⁺).

### Example 35

### Synthesis of N-(5-methyl-1,3,4-oxadiazol-2-yl)-2-[3-(trifluoromethoxy)phenyl]-7-azaspiro[3.5]nonane-7-carboxamide

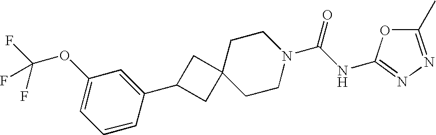

The title compound was prepared from 2-[3-(trifluoromethoxy)phenyl]-7-azaspiro[3.5]nonane hydrochloride (29.0 mg) and phenyl(5-methyl-1,3,4-oxadiazol-2-yl)carbamate (23.7 mg) as described for Example 12. The reaction was concentrated under a stream of nitrogen, dissolved in 1 mL DMSO, and purified by reverse phase HPLC (acetonitrile/water/0.1% formic acid) to give the title compound (2.15 mg). ¹H NMR (400 MHz, DMSO-d₆) δ ppm 7.45 (t, J=7.7 Hz, 1H), 7.30 (d, J=8.1 Hz, 1H), 7.15-7.21 (m, 2H), 3.53-3.65 (m, 1H), 3.45-3.52 (m, 2H), 3.31-3.40 (m, 2H), 2.38 (s, 3H), 2.23-2.33 (m, 2H), 1.81-1.90 (m, 2H), 1.65-1.71 (m, 2H), 1.48-1.53 (m, 2H). m/z 411 (MH⁺).

### Synthesis of 7-(tert-butoxycarbonyl)-7-azaspiro[3.5]nonane-2-carboxylic acid

tert-Butyl 2-cyano-7-azaspiro[3.5]nonane-7-carboxylate (1.5 g, 5.99 mmol) was dissolved in ethanol (40 mL) and water (40 mL). Lithium hydroxide (880 mg, 21 mmol) was added and the mixture was heated to reflux for 4 hours. The reaction mixture was cooled to room temperature. The ethanol was evaporated and the aqueous layer was acidified (pH 1-2) with 6N HCl. The aqueous layer was extracted with diethyl ether. The organic layer was washed with brine, dried (MgSO₄), filtered and concentrated to give the title compound as a white solid (1.6 g, 99%). ¹H NMR (400 MHz, CDCl₃) δ ppm 3.34-3.41 (m, 2H), 3.27-3.34 (m, 2H), 3.10-3.21 (m, 1H), 2.12 (d, J=8.9 Hz, 4H), 1.58-1.63 (m, 2H), 1.52-1.58 (m, 2H), 1.47 (s, 9H). m/z 214 (MH⁺ minus t-Bu), 292 (MNa⁺).

### Synthesis of tert-butyl 2-[methoxy(methyl)carbamoyl]-7-azaspiro[3.5]nonane-7-carboxylate

7-(tert-Butoxycarbonyl)-7-azaspiro[3.5]nonane-2-carboxylic acid (200 mg, 0.74 mmol) was suspended in dichloromethane (4 mL). To this suspension was added DIEA (0.25 mL, 1.5 mmol) and N,O-dimethylhydroxylamine hydrochloride (96 mg, 0.97 mmol). The mixture was cooled to 0° C. and HATU (339 mg, 0.89 mmol) was added. The mixture was gradually warmed to it and stirred under nitrogen overnight. The reaction mixture was washed with brine (3×), dried (MgSO₄), filtered and concentrated. The residue was purified on silica gel (60% ethyl acetate/heptane) to give the title compound as clear oil (150 mg, 65%). ¹H NMR (400 MHz, methanol-d₆) δ ppm 3.71 (s, 3H), 3.47-3.61 (m, 1H), 3.37-3.44 (m, 2H), 3.28-3.33 (m, 2H), 3.20 (s, 3H), 2.00-2.13 (m, 4H), 1.62-1.69 (m, 2H), 1.48-1.54 (m, 2H), 1.46 (s, 9H). m/z 257 (MH⁺−t-Bu).

### Synthesis of tert-butyl 2-acetyl-7-azaspiro[3.5]nonane-7-carboxylate

tert-Butyl 2-[methoxy(methyl)carbamoyl]-7-azaspiro[3.5]nonane-7-carboxylate (150 mg, 0.48 mmol) was dissolved in anhydrous THF and cooled to 0° C. Methylmagnesium bromide in diethyl ether (3 M, 0.16 mL, 0.48 mmol) was added dropwise. The reaction mixture was gradually warmed to room temperature and stirred overnight. The solution was cooled to 0° C. and additional methylmagnesium bromide (0.32 mL, 0.96 mmol) was added. The reaction mixture was warmed to room temperature. After stirring the reaction at room temperature for one hour the mixture was cooled once more to 0° C. and methylmagnesium bromide (0.32 mL, 0.96 mmoL) was added. The reaction mixture was warmed to room temperature and stirred for 2 hours. The reaction mixture was diluted with ethyl acetate and washed with brine. The aqueous layer was back extracted once with ethyl acetate. The combined organic layer was washed with aq satd ammonium chloride and then brine. The organic layer was dried (MgSO₄), filtered and concentrated. The residue was purified on silica gel (35% ethyl acetate/heptane) to give the title compound as a clear oil (110 mg, 86%). ¹H NMR (400 MHz, CDCl₃) δ ppm 3.33-3.39 (m, 2H), 3.24-3.30 (m, 2H), 3.13-3.24 (m, 1H), 2.10 (s, 3H), 2.00 (d, J=8.9 Hz, 4H), 1.55-1.62 (m, 2H), 1.41-1.48 (m, 11H). m/z 212 (MH⁺−t-Bu).

### Synthesis of tert-butyl 2-(bromoacetyl)-7-azaspiro[3.5]nonane-7-carboxylate

LDA (2M solution in heptane/THF/ethylbenzene 2.54 mL, 5.1 mmol) was added to 3 mL of anhydrous THF and cooled to −78° C. To this solution was added a solution of tert-butyl 2-acetyl 7-azaspiro[3.5]nonane-7-carboxylate (800 mg, 2.99 mmol) in anhydrous THF (2 mL). After 10 min TMSCl (0.66 mL, 5.1 mmol) was added dropwise. The reaction was gradually warmed to rt. After 45 min the reaction mixture was added to mixture of ether and aqueous sodium bicarbonate. The organic layer was dried (MgSO₄), filtered and concentrated. The residue was dissolved in anhydrous THF (1 mL) followed by addition of NaHCO₃ (253 mg, 2.99 mmol). The mixture was cooled to 0° C. and NBS (532 mg, 2.99 mmol) was added. The mixture was stirred at 0° C. for 90 min then diluted with ether and aqueous NaHCO₃. The organic layer was collected and the aqueous layer was extracted with 1× diethyl ether. The combined organic layer was washed with brine, dried (MgSO₄), filtered and concentrated. The residue was purified on silica gel (15% ethyl acetate/heptane) to give title compound as pale yellow solid (400 mg, 39%). ¹H NMR (400 MHz, CDCl₃) δ ppm 3.90 (s, 2H), 3.49-3.63 (m, 1H), 3.33-3.41 (m, 2H), 3.24-3.32 (m, 2H), 2.06 (d, J=8.5 Hz, 4H), 1.59-1.64 (m, 2H), 1.42-1.52 (m, 11H). m/z 292 (MH⁺−t-Bu).

### Synthesis of 2-{2-[3-(trifluoromethyl)phenyl]-1,3-thiazol-4-yl}-7-azaspiro[3.5]nonane trifluroacetate

tert-Butyl 2-(bromoacetyl)-7-azaspiro[3.5]nonane-7-carboxylate (200 mg, 0.57 mmol) was dissolved in ethanol followed by addition of 3-(trifluoromethyl)benzenecarbothioamide (90 mg, 0.57 mmol; CAS #53515-17-6). The mixture was heated to 80° C. for about 8 hours. The reaction mixture was cooled and the solvent was evaporated. The residue was treated with dichloromethane (4 mL)/TFA (1 mL) for 2 hours. The solvent was evaporated and the residue co-evaporated with methanol to give the title compound as an orange oil (340 mg) which was used without further purification. ¹H NMR (400 MHz, DMSO-d₆) δ ppm 8.16-8.24 (m, 2H), 7.81-7.90 (m, 1H), 7.75 (t, J=7.9 Hz, 1H), 7.52 (s, 1H), 3.61-3.75 (m, 1H), 2.92-3.13 (m, 4H), 2.30 (dd, J=11.9, 9.2 Hz, 2H), 2.08 (dd, J=11.9, 9.2 Hz, 2H), 1.83-1.91 (m, 2H), 1.71-1.80 (m, 2H). m/z 353 (MH⁺)

### Example 36

### Synthesis of N-(3,4-dimethylisoxazol-5-yl)-2-{2-[3-(trifluoromethyl)phenyl]-1,3-thiazol-4-yl}-7-azaspiro[3.5]nonane-7-carboxamide

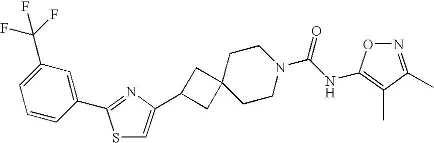

2-{2-[3-(Trifluoromethyl)phenyl]-1,3-thiazol-4-yl}-7-azaspiro[3.5]nonane trifluroacetate (340 mg, 0.59 mmol) was dissolved in acetonitrile (3 mL). To this solution was added DIEA (0.31 mL, 1.76 mmol) followed by phenyl(3,4-dimethylisoxazol-5-yl)carbamate (136 mg, 0.59 mmol). The mixture was stirred at room temperature overnight. The reaction mixture was concentrated and the residue was partitioned between ethyl acetate and brine. The ethyl acetate was washed with brine (2×), dried (MgSO₄), filtered and concentrated. The residue was purified twice by silica gel column chromatography (30% ethyl acetate/heptane) to give the title compound as an off-white solid (130 mg, 45%). ¹H NMR (400 MHz, CDCl₃) δ ppm 8.22 (s, 1H), 8.12 (d, J=7.5 Hz, 1H), 7.67 (d, J=7.9 Hz, 1H), 7.57 (t, J=7.9 Hz, 1H), 6.97 (s, 1H), 6.55 (s, 1H), 3.61-3.79 (m, 1H), 3.46-3.56 (m, 2H), 3.35-3.45 (m, 2H), 2.29-2.43 (m, 2H), 2.12-2.25 (m, 5H), 1.90 (s, 3H), 1.78-1.87 (m, 2H), 1.68-1.78 (m, 2H). m/z 491 (MH⁺).

### Example 37

### Synthesis of N-(3,4-dimethylisoxazol-5-yl)-2-[2-(3-fluorophenyl)-1,3-thiazol-4-yl]-7-azaspiro[3.5]nonane-7-carboxamide

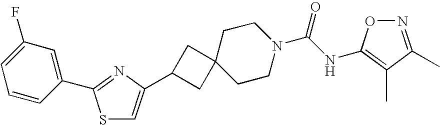

The title compound was prepared from tert-butyl 2-(bromoacetyl)-7-azaspiro[3.5]nonane-7-carboxylate (30 mg, 0.087 mmol), 3-fluorobenzenecarbothioamide (13.5 mg, 0.087 mmol; CAS #72505-20-5), and phenyl(3,4-dimethylisoxazol-5-yl)carbamate (20.2 mg, 0.087 mmol) as described for Example 36. The reaction was concentrated under a stream of nitrogen, dissolved in 1 mL DMSO, and purified by reverse phase HPLC (acetonitrile/water/0.1% formic acid) to give the title compound (13.22 mg). ¹H NMR (400 MHz, DMSO-d₆) δ ppm 9.09 (br. s., 1H), 7.69-7.80 (m, 2H), 7.51-7.59 (m, 1H), 7.45 (s, 1H), 7.29-7.36 (m, 1H), 3.62-3.74 (m, 1H), 3.30-3.47 (m, 4H), 2.22-2.31 (m, 2H), 2.12 (s, 3H), 2.01-2.10 (m, 2H), 1.73 (s, 3H), 1.64-1.71 (m, 2H), 1.53-1.63 (m, 2H) m/z 441.2 (MH⁺).

### Synthesis of tert-butyl 2-carbamoyl-7-azaspiro[3.5]nonane-7-carboxylate

7-(tert-Butoxycarbonyl)-7-azaspiro[3.5]nonane-2-carboxylic acid (1.0 g, 3.7 mmol) was dissolved in dichloromethane and cooled to 0° C. To this solution was added was added HATU (1.5 g, 4.1 mmol) followed by DIEA (0.97 mL, 5.6 mmol). After 45 min a solution of 7 N ammonia in methanol (0.58 mL, 4.1 mmol) was added. The reaction mixture was washed with brine followed by water. The organic layer was dried (MgSO₄), filtered and concentrated to give the title compound as a pale yellow solid (1.1 g, 110%). m/z 290.9 (MH⁺).

### Synthesis of tert-butyl 2-(aminocarbonothioyl)-7-azaspiro[3.5]nonane-7-carboxylate

tert-Butyl 2-carbamoyl-7-azaspiro[3.5]nonane-7-carboxylate (1.0 g, 3.7 mmol) and Lawesson's reagent (1.5 g, 3.7 mmol) were dissolved in toluene and stirred at 65° C. After 90 min the reaction mixture was cooled and the toluene was decanted from the solid that had formed during the reaction. The solid was washed 4× with toluene and the combined toluene solutions were concentrated. The residue was purified on silica gel eluting with 20% ethyl acetate/heptane to give the title compound. The material that precipitated during the reaction corresponding to the amine resulting from loss of the protecting group during the reaction was reprotected as follows. This material was dissolved in 20 mL of anhydrous THF. To this solution was added di-tert-butyl dicarbonate (810 mg, 3.7 mmol), DIEA (0.67 mL, 3.7 mmol) and DMAP (45 mg, 0.37 mmol). After stirring at rt overnight the reaction mixture was diluted with ethyl acetate and washed with brine. The organic layer was dried (MgSO₄), filtered and concentrated. The residue was purified on silica gel eluting with 15% ethyl acetate/heptane and combined with the first batch to give the title compound as a pale yellow solid (300 mg, 28.3%). m/z 229 (MH⁺ minus t-Bu).

### Example 38

### Synthesis of N-(3,4-dimethylisoxazol-5-yl)-2-{4-[4-(trifluoromethoxy)phenyl]-1,3-thiazol-2-yl}-7-azaspiro[3.5]nonane-7-carboxamide

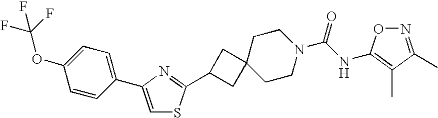

tert-Butyl 2-(aminocarbonothioyl)-7-azaspiro[3.5]nonane-7-carboxylate (35 mg, 0.12 mmol) and 2-bromo-1-[4-(trifluoromethoxy)phenyl]ethanone were dissolved in ethanol and heated to 80° C. After 3 hours the reaction mixture was cooled and the solvent was evaporated to give the crude 2-{4-[4-(trifluoromethoxy)phenyl]-1,3-thiazol-2-yl}-7-azaspiro[3.5]nonane. The residue was dissolved in acetonitrile followed by addition of DIEA (0.021 mL, 0.12 mmoL) and phenyl(3,4-dimethylisoxazol-5-yl)carbamate (34.8 mg, 0.12 mmol). After 90 min, the reaction was concentrated, dissolved in 1 mL DMSO and purified by reverse phase HPLC (acetonitrile/water/0.1% formic acid) to give the title compound (8.21 mg, 13.7%). ¹H NMR (400 MHz, DMSO-d₆) δ ppm 9.08 (br. s., 1H), 8.03-8.09 (m, 3H), 7.42 (d, J=8.1 Hz, 2H), 3.86-3.99 (m, 1H), 3.41 (d, J=5.1 Hz, 2H), 3.29-3.35 (m, 2H), 2.32-2.44 (m, 2H), 2.03-2.14 (m, 5H), 1.72 (s, 3H), 1.64-1.71 (m, 2H), 1.50-1.60 (m, 2H) m/z 507.2 (MH⁺).

### Example 39

### Synthesis of 2-(4-chloro-3-fluorophenyl)-N-(3,4-dimethylisoxazol-5-yl)-7-azaspiro[3.5]nonane-7-carboxamide

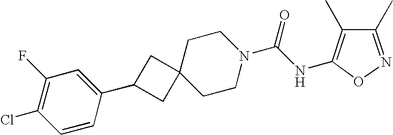

The title compound was prepared from 2-bromo-7-aza-spiro[3.5]nonane-7-carboxylic acid tert-butyl ester, (4-chloro-3-fluorophenyl)boronic acid (CAS #137504-86-0), and phenyl(3,4-dimethylisoxazol-5-yl)carbamate as described for Example 40 to give the title compound (8.96 mg). LCMS (Phenomenex Gemini C18 4.6×50 mm 5 μm (0.04% Formic Acid, 0.01% TFA/MeCN)) t_{R} 2.11 min; m/z 392.55 (MH⁺).

### Example 40

### Synthesis of N-(3,4-dimethylisoxazol-5-yl)-2-{4-[(4-fluorobenzyl)oxy]phenyl}-7-azaspiro[3.5]nonane-7-carboxamide

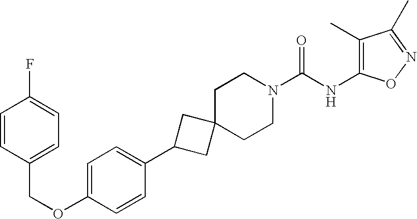

A 0.4 M stock solution of 2-bromo-7-aza-spiro[3.5]nonane-7-carboxylic acid tert-butyl ester in anhydrous isopropanol (0.5 mL, 0.200 mmol) and a 0.024 M stock solution of trans 2-aminocyclohexanol in isopropanol (0.5 mL, 0.012 mmol, 0.06 equiv) was added to a vial containing sodium hexamethyldisilazide (0.400 mmol, 2 equiv), nickel iodide (0.012 mmol, 0.06 equiv), and {4-[(4-fluorobenzyl)oxy]phenyl}boronic acid (0.400 mmol, 2 equiv; CAS #871125-82-5). The vial was flushed with nitrogen, capped, and shook at 70° C. overnight. The reaction mixtures were concentrated under vacuum to give the crude tert-butyl carbamate derivative. The residue was dissolved in dichloromethane (1.2 mL) and treated with 4 N HCl in dioxane (0.8 mL). After shaking for 2 h, the reaction mixture was concentrated under vacuum to give the crude amine hydrochloride salt derivative. The crude amine hydrochloride salt residue was dissolved in acetonitrile (2.0 mL) and split into two separate vials (1.0 mL, 0.1 mmol each). To the solution in one of the vials was added diisopropylethylamine (0.17 mL, 1.0 mmol, 10 equiv) followed by a solution of phenyl(3,4-dimethylisoxazol-5-yl)carbamate (0.120 mmol in acetonitrile, 1.2 equiv). After shaking overnight at rt, the reaction was concentrated under vacuum, diluted in DMSO (1.5 mL), filtered through celite, and purified by reverse phase HPLC (acetonitrile/water/0.05% trifluoroacetic acid) to give the title compound (8.5 mg). LCMS (Phenomenex Gemini C18 4.6×50 mm 5 μm (0.04% Formic Acid, 0.01% TFA/MeCN)) t_{R} 2.23 min; m/z 464.45 (MH⁺).

### Synthesis of tert-butyl 2-(3-chloro-4-fluorophenyl)-2-hydroxy-1-methyl-7-azaspiro[3.5]nonane-7-carboxylate

To a solution of diisopropylamide (0.358 mL, 2.51 mmol) in THF (4 mL) at 0° C. under nitrogen was added n-BuLi (0.920 mL, 2.30 mmol) and the solution stirred for 10 minutes. To this solution was added tert-butyl 2-oxo-7-azaspiro[3.5]nonane-7-carboxylate (500 mg, 2.09 mmol) dropwise in THF (2 mL) and the solution stirred for an addition 2 hours at this temp. Methyl Iodide (0.157 mL, 2.51 mmol) was then added and the solution allowed to gradually warm to r.t. The reaction was quenched with saturated ammonium chloride and the aqueous phase extracted with ethyl acetate (3×10 mL). The organics were dried with magnesium sulfate and concentrated to give a 1:1:1 mixture of the title compound along with the dialkylated derivate and unreacted starting material. The crude mixture was dissolved in THF (2 mL) and cooled to 0° C. 3-Chloro-4-fluorophenylmagnesium bromide (0.616 mL, 0.308 mmol) was added dropwise and the solution stirred for 1 hour at r.t. The reaction was quenched with saturated ammonium chloride and the aqueous phase extracted with ethyl acetate (3×10 mL). The organics were dried with magnesium sulfate and concentrated. The crude product was purified by flash chromatography (10% ethyl acetate/heptanes) to give the title compound as an oil (60 mg, 0.156 mmol, 7%).

### Example 41

### Synthesis of 2-(3-chloro-4-fluorophenyl)-N-(3,4-dimethylisoxazol-5-yl)-1-methyl-7-azaspiro[3.5]nonane-7-carboxamide

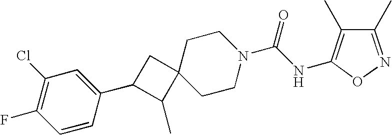

To a solution of tert-butyl 2-(3-chloro-4-fluorophenyl)-2-hydroxy-1-methyl-7-azaspiro[3.5]nonane-7-carboxylate (35 mg, 0.077 mmol) in dichloromethane (1 mL) at 0° C. was added triethylsilane (0.052 mL, 0.32 mmol), borontrifluoride diethyletherate (0.019 mL, 0.154 mmol), trifluoroacetic acid (0.029 mL, 0.39 mL) and the solution stirred for 1 hour at r.t. The reaction was quenched with saturated sodium bicarbonate and the aqueous phase extracted with dichloromethane. The organics were dried with magnesium sulfate and concentrated. The crude amine was dissolved in acetonitrile (1 mL) and phenyl(3,4-dimethylisoxazol-5-yl)carbamate (13 mg, 0.056 mmol) and triethylamine (0.080 mL, 0.616 mmol, 4.0 equiv) added and the resulting solution stirred for 1 h at room temp. The reaction was concentrated, dissolved in 1 mL DMSO and purified by reverse phase HPLC (acetonitrile/water/0.1% formic acid) to give the title compound as a mixture of stereoisomers (5.9 mg). ¹H NMR (400 MHz, DMSO-d₆) δ ppm 9.06 (1H, br. s.), 7.35-7.45 (1H, m), 7.26-7.35 (1H, m), 7.11-7.26 (1H, m), 3.87-4.07 (1H, m), 3.69-3.85 (1H, m), 2.77-3.08 (2H, m), 2.50-2.54 (1H, m), 2.05-2.14 (3H, m), 1.87 (2H, s), 1.61-1.77 (4H, m), 1.31-1.55 (3H, m), 0.89-1.04 (2H, m). m/z 406.2 (MH⁺).

### Synthesis of tert-butyl 3-methyl-4-oxopiperidine-1-carboxylate

In a high pressure vessel was added 1-benzyl-3-methyl-piperidin-4one (6.0 g, 29.52 mmol) which was dissolved in methanol (60 mL). Di-tertbutyl dicarbonate (8.37 g, 38.40 mmol) was added along with palladium hydroxide (829 mg, 5.90 mmol) and the reaction stirred at r.t. overnight under 55 psi hydrogen atmosphere. The reaction was vacuum filtered through celite and concentrated. The crude material was purified by flash chromatography (10% ethyl acetate/heptanes) to give the title compound as a colorless oil (6.01 g, 28.21 mmol, 95%).

### Synthesis of tert-butyl 3-methyl-4-methylenepiperidine-1-carboxylate

Methyltriphenylphosphonium bromide (13.70 g, 38.40 mmol) was dissolved in THF (100 mL) and cooled to 0° C. n-BuLi (17.7 mL, 44.3 mmol) was added dropwise and the solution stirred for 30 minutes. At this time tert-butyl 3-methyl-4-oxopiperidine-1-carboxylate (6.29 g, 29.52 mmol) was added and the solution stirred at r.t. for 1 hour. The reaction was quenched with saturated ammonium chloride and the aqueous phase extracted with ethyl acetate (2×50 mL). The organics were dried with magnesium sulfate and dried. The crude material was purified by flash chromatography (10% ethyl acetate/heptanes) to give the title compound as a colorless oil (5.20 g, 24.64 mmol, 83%).

### Synthesis of tert-butyl 5-methyl-2-oxo-7-azaspiro[3.5]nonane-7-carboxylate

To a stirred suspension of zinc-copper couple (25.10 g, 193 mmol) and tert-butyl 3-methyl-4-methylenepiperidine-1-carboxylate (6.0 g, 28.40 mmol) in diethyl ether (180 mL) was added a solution of trichloroacetyl chloride (17.3 mL, 153 mmol) in DME (100 mL) very slowly dropwise and the mixture stirred for 3 hours. The reaction was quenched by careful addition of the mixture to ice and satd sodium bicarbonate (˜1000 mL). The mixture was filtered, washing the cake with ethyl acetate (100 mL). The filtrate was extracted with ethyl acetate (2×50 mL). The organic layers were washed with brine, dried with sodium sulfate and concentrated. The crude material was dissolved in saturated ammonium chloride in methanol (100 mL) followed by Zn (15.20 g, 232 mmol) addition in one portion. The reaction was stirred for 6 hours at r.t. At this time the reaction was filtered through celite, concentrated and the crude material was purified by flash chromatography (10% ethyl acetate/heptanes) to give the title compound as a white solid (5.46 g, 21.58 mmol, 76%).

### Synthesis of 2-(3-chloro-4-fluorophenyl)-5-methyl-7-azaspiro[3.5]nonane

The title compound was prepared from 3-chloro-4-fluorophenylmagnesium bromide (0.5 M solution in THF; Aldrich) and tert-butyl 5-methyl-2-oxo-7-azaspiro[3.5]nonane-7-carboxylate (1.50 g) as described for 2-[3-(trifluoromethoxy)phenyl]-7-azaspiro[3.5]nonane hydrochloride. The crude title compound was obtained as an oil (800 mg) and not converted to the hydrochloride salt.

### Example 42

### Synthesis of 2-(3-chloro-4-fluorophenyl)-N-(3,4-dimethylisoxazol-5-yl)-5-methyl-7-azaspiro[3.5]nonane-7-carboxamide

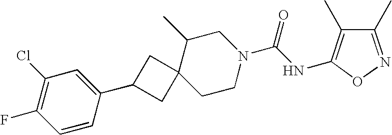

The title compound was prepared from 2-(3-chloro-4-fluorophenyl)-5-methyl-7-azaspiro[3.5]nonane (800 mg) and phenyl(3,4-dimethylisoxazol-5-yl)carbamate (694 mg) as described for Example 12. The crude product was purified by flash chromatography (ethyl acetate/heptane) to give the title compound as a mixture of stereoisomers (650 mg, 54%). ¹H NMR (400 MHz, DMSO-d₆) δ ppm 9.03 (1H, br. s.), 7.33-7.44 (1H, m), 7.29 (1H, t, J=8.9 Hz), 7.14-7.25 (1H, m), 3.26 (2H, br. s.), 3.14 (2H, br. s.), 2.48 (3H, br. s.), 2.37 (2H, d, J=9.3 Hz), 2.08 (3H, s), 1.52-1.74 (6H, m), 0.96 (2H, d, J=7.0 Hz), 0.79 (1H, d, J=7.0 Hz). m/z 406.2 (MH⁺).

The stereoisomers of 2-(3-chloro-4-fluorophenyl)-N-(3,4-dimethylisoxazol-5-yl)-5-methyl-7-azaspiro[3.5]nonane-7-carboxamide (650 mg in 22 mL methanol) were separated by chiral SFC on a Chiralpak AS-H column (30×250 mm; 30% isopropanol/CO₂; 1 ml/injection) to give the following four isomers.

Example 42a. 2-(3-chloro-4-fluorophenyl)-N-(3,4-dimethylisoxazol-5-yl)-5-methyl-7-azaspiro[3.5]nonane-7-carboxamide (Isomer 1). First eluting peak, 117 mg, t_{R}=3.04 min (4.6×150 mm Chiralpak AS-H, 50% isopropanol/CO₂ at 3 mL/min).

Example 42b. 2-(3-chloro-4-fluorophenyl)-N-(3,4-dimethylisoxazol-5-yl)-5-methyl-7-azaspiro[3.5]nonane-7-carboxamide (Isomer 2). Second eluting peak, 71 mg, t_{R}=3.68 min (4.6×150 mm Chiralpak AS-H, 50% isopropanol/CO₂ at 3 mL/min).

Example 42c. 2-(3-chloro-4-fluorophenyl)-N-(3,4-dimethylisoxazol-5-yl)-5-methyl-7-azaspiro[3.5]nonane-7-carboxamide (Isomer 3). Third eluting peak, 100 mg, t_{R}=4.45 min (4.6×150 mm Chiralpak AS-H, 50% isopropanol/CO₂ at 3 mL/min).

Example 42d. 2-(3-chloro-4-fluorophenyl)-N-(3,4-dimethylisoxazol-5-yl)-5-methyl-7-azaspiro[3.5]nonane-7-carboxamide (Isomer 4). Fourth eluting peak, 45 mg, t_{R}=5.97 min (4.6×150 mm Chiralpak AS-H, 50% isopropanol/CO₂ at 3 mL/min).

### Synthesis of 2-(3-chloro-4-fluorophenyl)-2-methoxy-7-azaspiro[3.5]nonane hydrochloride

A solution of tert-butyl 2-(3-chloro-4-fluorophenyl)-2-hydroxy-7-azaspiro[3.5]nonane-7-carboxylate (300 mg, 0.811 mmol, 1 equiv) in DMF (6 mL) was treated with sodium hydride (60% dispersion in mineral oil, 35.7 mg, 0.892 mmol, 1.1 equiv) at 0° C. The mixture was allowed to warm to room temp for 15 min, and then iodomethane (0.0555 mL, 0.892 mmol, 1.1 equiv) was added. The mixture was stirred at room temp overnight and then was quenched with said ammonium chloride and extracted with ethyl acetate. The organic layers were dried over sodium sulfate, filtered, and concentrated to give crude tert-butyl 2-(3-chloro-4-fluorophenyl)-2-methoxy-7-azaspiro[3.5]nonane-7-carboxylate as a clear oil (307 mg). The crude oil was dissolved in dichloromethane (4 mL) and treated with 4 N HCl/dioxane (1 mL) at room temp for 1 h. The reaction was concentrated and dried under vacuum to give the title compound as a white solid (268 mg, quant). m/z 284 (MH⁺).

### Example 43

### Synthesis of 2-(3-chloro-4-fluorophenyl)-N-(3,4-dimethylisoxazol-5-yl)-2-methoxy-7-azaspiro[3.5]nonane-7-carboxamide

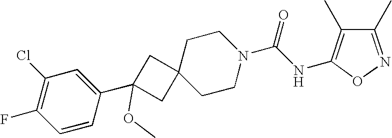

The title compound was prepared from 2-(3-chloro-4-fluorophenyl)-2-methoxy-7-azaspiro[3.5]nonane hydrochloride (25.3 mg) and phenyl(3,4-dimethylisoxazol-5-yl)carbamate (22.1 mg) as described for Example 12. The crude reaction mixture was concentrated, dissolved in 1 mL DMSO and purified by reverse phase HPLC (acetonitrile/water/0.1% formic acid) to give the title compound (20 mg). ¹H NMR (400 MHz, DMSO-d₆) δ ppm 9.07 (s, 1H), 7.53 (dd, J=7.3, 2.2 Hz, 1H), 7.36-7.46 (m, 2H), 3.37-3.42 (m, 2H), 3.27-3.33 (m, 2H), 2.82 (s, 3H), 2.17-2.33 (m, 4H), 2.12 (s, 3H), 1.73 (s, 3H), 1.63-1.69 (m, 2H), 1.35-1.41 (m, 2H). m/z 422 (MH⁺).

### Example 44

### Synthesis of 2-(3-chloro-4-fluorophenyl)-N-(3,4-dimethylisoxazol-5-yl)-2-hydroxy-7-azaspiro[3.5]nonane-7-carboxamide

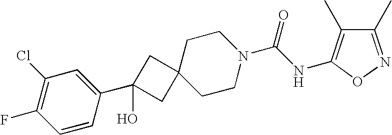

A solution of tert-butyl 2-(3-chloro-4-fluorophenyl)-2-hydroxy-7-azaspiro[3.5]nonane-7-carboxylate (33.7 mg, 0.091 mmol) in dichloromethane (0.4 mL) was treated with 4 N HCl/dioxane (0.15 mL) at room temp for 1 h. The mixture was concentrated under a stream of nitrogen to give the crude amine. A mixture of the amine and phenyl(3,4-dimethylisoxazol-5-yl)carbamate (25.3 mg) in acetonitrile (0.5 mL) was treated with DIEA (0.111 mL) and stirred for 1.5 h at room temp. The reaction was concentrated under a stream of nitrogen, dissolved in 1 mL DMSO and purified by reverse phase HPLC (acetonitrile/water/0.1% formic acid) to give the title compound (25 mg). ¹H NMR (400 MHz, DMSO-d₆) δ ppm 9.08 (s, 1H), 7.56 (dd, J=7.3, 2.2 Hz, 1H), 7.42-7.47 (m, 1H), 7.38 (t, J=8.8 Hz, 1H), 5.63 (br. s., 1H), 3.38-3.43 (m, 2H), 3.29-3.34 (m, 2H), 2.29 (d, J=13.2 Hz, 2H), 2.10-2.17 (m, 2H), 2.12 (s, 3H), 1.76-1.81 (m, 2H), 1.73 (s, 3H), 1.45-1.50 (m, 2H). m/z 408 (MH⁺).

### Synthesis of 4-nitrophenyl 2-[3-(trifluoromethoxy)phenyl]-7-azaspiro[3.5]nonane-7-carboxylate

Title compound was prepared from 2-[3-(trifluoromethoxy)phenyl]-7-azaspiro[3.5]nonane HCl (3.32 g) as described for 4-nitrophenyl 2-(3-methylphenyl)-7-azaspiro[3.5]nonane-7-carboxylate. The reaction suspension was partitioned between ethyl acetate and ½ satd sodium bicarbonate. The organic extract washed several times with said sodium bicarbonate and brine, dried over sodium sulfate, filtered, concentrated and purified by flash chromatography (10 to 30% ethyl acetate/heptane) to give the title compound as a white solid (4.26 g, 92%). m/z 451 (MH⁺).

### Example 45

### Synthesis of N-(1-methyl-1H-tetrazol-5-yl)-2-[3-(trifluoromethoxy)phenyl]-7-azaspiro[3.5]nonane-7-carboxamide

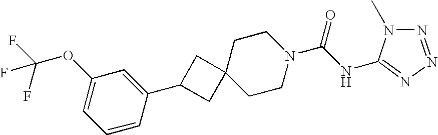

A 0.5 M solution of the sodium salt of 1-methyl-1H-tetrazol-5-amine was prepared by the portionwise addition of sodium hydride (60% dispersion in mineral oil, 410 mg, 10.2 mmol) to a solution of 1-methyl-1H-tetrazol-5-amine (991 mg, 10 mmol; CAS #5422-44-6) in DMA (20 mL). The suspension was stirred at room temp for 10 min. 4.5 mL of the 0.5 M solution of the sodium salt of 1-methyl-1H-tetrazol-5-amine (2.22 mmol, 2 eq) was added to 4-nitrophenyl 2-[3-(trifluoromethoxy)phenyl]-7-azaspiro[3.5]nonane-7-carboxylate (500 mg) in DMA (1.5 mL) and stirred at room temp overnight. The reaction was quenched with water, diluted with ethyl acetate and washed with ½ saturated sodium bicarbonate, ½ satd sodium carbonate, water, and brine. The organic layers were dried over sodium sulfate, filtered, concentrated, and purified by flash chromatography (30 to 70% ethyl acetate/heptane). The product was recrystallized from ethyl acetate/heptane to give the title compound as a white solid (280 mg, 60%). ¹H NMR (400 MHz, DMSO-d₆) δ ppm 9.88 (s, 1H), 7.46 (t, J=7.7 Hz, 1H), 7.31 (d, J=8.1 Hz, 1H), 7.16-7.23 (m, 2H), 3.81 (s, 3H), 3.56-3.68 (m, 1H), 3.47-3.54 (m, 2H), 3.36-3.42 (m, 2H), 2.27-2.37 (m, 2H), 1.85-1.94 (m, 2H), 1.72-1.78 (m, 2H), 1.54-1.61 (m, 2H). m/z 411 (MH⁺).

### Synthesis of 4-nitrophenyl 2-(3-chloro-2-fluorophenyl)-7-azaspiro[3.5]nonane-7-carboxylate

Title compound was prepared from 2-(3-chloro-2-fluorophenyl)-7-azaspiro[3.5]nonane HCl (500 mg) as described for 4-nitrophenyl 2-(3-methylphenyl)-7-azaspiro[3.5]nonane-7-carboxylate. The reaction suspension was partitioned between ethyl acetate and ½ satd sodium bicarbonate. The organic extract washed several times with satd sodium bicarbonate and brine, dried over sodium sulfate, filtered, concentrated and purified by flash chromatography (5 to 25% ethyl acetate/heptane) to give the title compound as a white solid (469 mg, 65%). m/z 419 (MH⁺).

### Example 46

### Synthesis of 2-(3-chloro-2-fluorophenyl)-N-(1-methyl-1H-tetrazol-5-yl)-7-azaspiro[3.5]nonane-7-carboxamide

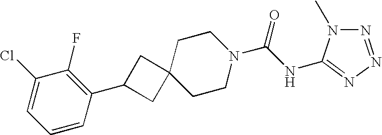

The title compound was prepared from 4-nitrophenyl 2-(3-chloro-2-fluorophenyl)-7-azaspiro[3.5]nonane-7-carboxylate (459 mg) and as described for Example 45. The chromatographed product was suspended in boiling ethyl acetate, cooled to room temp, and filtered to give the title compound as a white solid (297 mg, 72%). ¹H NMR (400 MHz, DMSO-d₆) δ ppm 9.87 (br. s., 1H), 7.42 (t, J=7.3 Hz, 1H), 7.36 (t, J=7.3 Hz, 1H), 7.21 (t, J=8.1 Hz, 1H), 3.81 (s, 3H), 3.69-3.79 (m, 1H), 3.47-3.55 (m, 2H), 3.34-3.43 (m, 2H), 2.33 (t, J=10.3 Hz, 2H), 1.91-2.00 (m, 2H), 1.74-1.81 (m, 2H), 1.52-1.59 (m, 2H). m/z 379 (MH⁺).

### Synthesis of 4-nitrophenyl 2-[2-fluoro-3-(trifluoromethyl)phenyl]-7-azaspiro[3.5]nonane-7-carboxylate

The title compound was prepared from 2-[2-fluoro-3-(trifluoromethyl)phenyl]-7-azaspiro[3.5]nonane hydrochloride (558 mg) as described for 4-nitrophenyl 2-(3-methylphenyl)-7-azaspiro[3.5]nonane-7-carboxylate. The reaction suspension was partitioned between ethyl acetate and ½ satd sodium bicarbonate. The organic extract washed several times with said sodium bicarbonate and brine, dried over sodium sulfate, filtered, concentrated and purified by flash chromatography (5 to 25% ethyl acetate/heptane) to give the title compound as an off-white waxy solid (507 mg, 65%). m/z 453 (MH⁺).

### Example 47

### Synthesis of 2-[2-fluoro-3-(trifluoromethyl)phenyl]-N-(1-methyl-1H-tetrazol-5-yl)-7-azaspiro[3.5]nonane-7-carboxamide

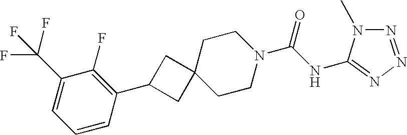

The title compound was prepared from 4-nitrophenyl 2-[2-fluoro-3-(trifluoromethyl)phenyl]-7-azaspiro[3.5]nonane-7-carboxylate (496 mg) as described for Example 45. The chromatographed product was suspended in boiling ethyl acetate, cooled to room temp, and filtered to give the title compound as a white solid (326 mg, 72%). ¹H NMR (400 MHz, DMSO-d₆) δ ppm 9.88 (br. s., 1H), 7.74 (t, J=7.7 Hz, 1H), 7.62 (t, J=7.0 Hz, 1H), 7.40 (t, J=7.7 Hz, 1H), 3.81 (s, 3H), 3.75-3.86 (m, 1H), 3.49-3.55 (m, 2H), 3.36-3.42 (m, 2H), 2.35 (d, J=9.5 Hz, 2H), 1.99 (t, J=11.0 Hz, 2H), 1.74-1.81 (m, 2H), 1.53-1.61 (m, 2H). m/z 413 (MH⁺).

### Synthesis of 4-nitrophenyl 2-(3-chloro-4-fluorophenyl)-7-azaspiro[3.5]nonane-7-carboxylate

Title compound was prepared from 2-(3-chloro-4-fluorophenyl)-7-azaspiro[3.5]nonane hydrochloride (2.10 g) as described for 4-nitrophenyl 2-(3-methylphenyl)-7-azaspiro[3.5]nonane-7-carboxylate. The reaction suspension was partitioned between ethyl acetate and ½ saturated sodium bicarbonate. The organic extract washed several times with saturated sodium bicarbonate and brine, dried over sodium sulfate, filtered, concentrated and purified by flash chromatography (10 to 30% ethyl acetate/heptane) to give the title compound as an off-white solid (1.18 g, 34%). m/z 419 (MH⁺).

### Example 48

### Synthesis of 2-(3-chloro-4-fluorophenyl)-N-(1-methyl-1H-tetrazol-5-yl)-7-azaspiro[3.5]nonane-7-carboxamide

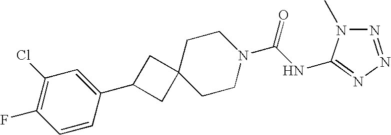

The title compound was prepared from 4-nitrophenyl 2-(3-chloro-4-fluorophenyl)-7-azaspiro[3.5]nonane-7-carboxylate (510 mg) as described for Example 45. The chromatographed product was suspended in boiling ethyl acetate, cooled to room temp, and filtered to give the title compound as a white solid (287 mg, 62%). ¹H NMR (400 MHz, DMSO-d₆) δ ppm 9.86 (br. s., 1H), 7.45 (dd, J=7.3, 2.2 Hz, 1H), 7.34 (t, J=8.8 Hz, 1H), 7.25-7.30 (m, 1H), 3.81 (s, 3H), 3.52-3.62 (m, 1H), 3.47-3.53 (m, 2H), 3.35-3.41 (m, 2H), 2.25-2.33 (m, 2H), 1.84-1.91 (m, 2H), 1.70-1.76 (m, 2H), 1.54-1.60 (m, 2H). m/z 379 (MH⁺).

### Synthesis of 4-nitrophenyl 2-{5-[4-(trifluoromethyl)phenyl]-1,2,4-oxadiazol-3-yl}-7-azaspiro[3.5]nonane-7-carboxylate

2-{5-[4-(Trifluoromethyl)phenyl]-1,2,4-oxadiazol-3-yl}-7-azaspiro[3.5]nonane trifluoroacetate (150 mg, 0.44 mmol) was dissolved in anhydrous dioxane (4 mL) followed by addition of DIEA (0.23 mL, 1.3 mmol). 4-Nitrophenyl chlorocarbonate (90 mg, 0.44 mmol) was dissolved in dioxane (1 mL) and added dropwise at room temperature. The mixture was stirred overnight. The mixture was concentrated redissolved in dioxane (˜3 mL) and a saturated aqueous solution of sodium bicarbonate was added. After stirring overnight the mixture was extracted with ethyl acetate. The ethyl acetate was washed with sodium bicarbonate, dried (MgSO₄), filtered and concentrated. The residue was purified on silica gel (20% ethyl acetate/heptane) to give the title compound as a semi solid (170 mg, 76%). ¹H NMR (400 MHz, CDCl₃) δ ppm 8.44 (s, 1H), 8.35 (d, J=7.9 Hz, 1H), 8.22-8.30 (m, 2H), 7.89 (d, J=7.9 Hz, 1H), 7.72 (t, J=7.9 Hz, 1H), 7.26-7.35 (m, 2H), 3.74-3.86 (m, 1H), 3.48-3.74 (m, 4H), 2.28-2.48 (m, 4H), 1.77-1.94 (m, 4H). m/z 503 (MH⁺).

### Example 49

### Synthesis of N-(1-methyl-1H-tetrazol-5-yl)-2-{5-[4-(trifluoromethyl)phenyl]-1,2,4-oxadiazol-3-yl}-7-azaspiro[3.5]nonane-7-carboxamide

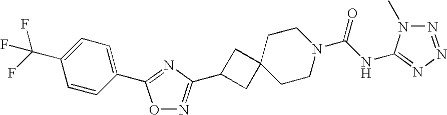

1-Methyl-1H-tetrazol-5-amine (67 mg, 0.68 mmol) was dissolved in 2 mL of anhydrous DMA. To this solution was added NaH (27 mg, 0.68 mmol) in portions. After 15 minutes this solution was added to a solution of 4-nitrophenyl 2-{5-[4-(trifluoromethyl)phenyl]-1,2,4-oxadiazol-3-yl}-7-azaspiro[3.5]nonane-7-carboxylate (170 mg, 0.34 mmol) in DMA (1 mL). The reaction mixture was stirred overnight at room temperature. An additional 2 eq of the sodium of 1-methyl-1H-tetrazol-5-amine (67 mg of 1-methyl-1H-tetrazol-5-amine; 27 mg of NaH) was added to the reaction mixture and stirred at room temperature for 2 hours. The reaction mixture was diluted with ethyl acetate and washed with half saturated sodium bicarbonate, water and brine. The organic layer was dried (MgSO₄), filtered and concentrated. The residue was purified on silica gel (60% ethyl acetate/heptane increasing polarity to 1% methanol/ethyl acetate). The purified material was recrystallized from ethyl acetate/heptane to give the title compound as an off-white solid (30 mg, 19%). ¹H NMR (400 MHz, DMSO-d₆) δ ppm 8.42 (d, J=8.2 Hz, 1H), 8.30-8.37 (m, 1H), 8.11 (d, J=7.9 Hz, 1H), 7.90 (t, J=7.9 Hz, 1H), 4.03 (q, J=7.2 Hz, 1H), 3.79 (s, 3H), 3.49 (d, J=10.2 Hz, 2H), 3.40 (d, J=10.6 Hz, 2H), 2.27-2.39 (m, 2H), 2.13-2.23 (m, 2H), 1.70-1.79 (m, 2H), 1.57-1.67 (m, 2H). m/z 463 (MH⁺).

### Synthesis of 4-nitrophenyl 2-{5-[4-(trifluoromethoxy)phenyl]-1,2,4-oxadiazol-3-yl}-7-azaspiro[3.5]nonane-7-carboxylate

The title compound was prepared from 2-{5-[4-(trifluoromethoxy)phenyl]-1,2,4-oxadiazol-3-yl}-7-azaspiro[3.5]nonane trifluoroacetate (240 mg, 0.32 mmol) and 4-nitrophenyl chloroformate (71 mg, 0.35 mmol) in the same manner as described for 2-{5-[4-(trifluoromethyl)phenyl]-1,2,4-oxadiazol-3-yl}-7-azaspiro[3.5]nonane-7-carboxylate. The crude compound was purified on silica gel (15% ethyl acetate/heptane) to give the title compound as a white solid (110 mg, 66%). ¹H NMR (400 MHz, DMSO-d₆) δ ppm 8.27 (t, J=9.4 Hz, 4H), 7.64 (d, J=8.9 Hz, 2H), 7.44 (d, J=8.5 Hz, 2H), 3.72-3.85 (m, 1H), 3.36-3.66 (m, 4H), 2.30-2.40 (m, 2H), 2.13-2.22 (m, 2H), 1.76-1.85 (m, 2H), 1.62-1.74 (m, 2H). m/z 519 (MH⁺).

### Example 50

### Synthesis of N-(1-methyl-1H-tetrazol-5-yl)-2-{5-[4-(trifluoromethoxy)phenyl]-1,2,4-oxadiazol-3-yl}-7-azaspiro[3.5]nonane-7-carboxamide

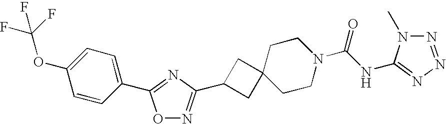

The title compound was prepared from 4-nitrophenyl 2-{5-[4-(trifluoromethoxy)phenyl]-1,2,4-oxadiazol-3-yl}-7-azaspiro[3.5]nonane-7-carboxylate (100 mg, 0.19 mmol) and 1-methyl-1H-tetrazol-5-amine (47.9 mg, 0.48 mmol) in the same manner as described for Example 49. The crude product was purified on silica gel (60-100% ethyl acetate/heptane) and then triturated with diethyl ether to give the title compound as a tan solid (25 mg, 27%). ¹H NMR (400 MHz, DMSO-d₆) δ ppm 8.42 (d, J=8.2 Hz, 1H), 8.35 (s, 1H), 8.11 (d, J=7.9 Hz, 1H), 7.90 (t, J=7.9 Hz, 1H), 4.03 (q, J=7.2 Hz, 1H), 3.79 (s, 3H), 3.49 (d, J=10.2 Hz, 2H), 3.40 (d, J=10.6 Hz, 2H), 2.27-2.40 (m, 2H), 2.12-2.23 (m, 2H), 1.71-1.80 (m, 2H), 1.59-1.68 (m, 2H). m/z 479 (MH⁺).

### Example 51

### Synthesis of N-[5-(3-chlorophenyl)-1,3,4-oxadiazol-2-yl]-2-[3-(trifluoromethoxy)phenyl]-7-azaspiro[3.5]nonane-7-carboxamide

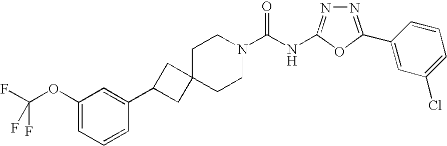

The title compound was prepared from a 0.16 M solution of 4-nitrophenyl 2-[3-(trifluoromethoxy)phenyl]-7-azaspiro[3.5]nonane-7-carboxylate in DMA (0.5 mL, 0.080 mmol) and 5-(3-chlorophenyl)-1,3,4-oxadiazol-2-amine (31.3 mg, 0.16 mmol; CAS #1673-45-6) as described for Example 16. The crude residue was dissolved in 1 mL DMSO and purified by reverse phase HPLC (acetonitrile/water/0.1% formic acid) to give the title compound (2.3 mg). ¹H NMR (400 MHz, DMSO-d₆) δ ppm 7.72-7.78 (m, 3H), 7.42-7.60 (m, 4H), 7.38 (s, 1H), 7.30 (d, J=8.1 Hz, 1H), 7.15-7.21 (m, 2H), 3.51-3.65 (m, 3H), 3.41-3.46 (m, 2H), 1.80-1.87 (m, 2H), 1.60-1.64 (m, 2H), 1.41-1.46 (m, 2H). m/z 507 (MH⁺).

### Example 52

### Synthesis of N-(1-ethyl-1H-tetrazol-5-yl)-2-[3-(trifluoromethoxy)phenyl]-7-azaspiro[3.5]nonane-7-carboxamide

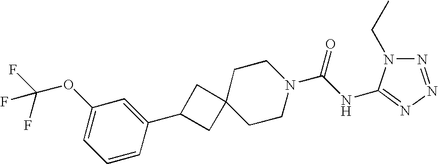

The title compound was prepared from a 0.16 M solution of 4-nitrophenyl 2-[3-(trifluoromethoxy)phenyl]-7-azaspiro[3.5]nonane-7-carboxylate in DMA (0.5 mL, 0.080 mmol) and 1-ethyl-1H-tetrazol-5-amine (18.1 mg, 0.16 mmol; CAS #65258-53-9) as described for Example 16. The crude residue was dissolved in 1 mL DMSO and purified by reverse phase HPLC (acetonitrile/water/0.1% formic acid) to give the title compound (3.95 mg). ¹H NMR (400 MHz, DMSO-d₆) δ ppm 9.80 (br. s., 1H), 7.45 (t, J=7.7 Hz, 1.H), 7.31 (d, J=7.3 Hz, 1H), 7.16-7.23 (m, 2H), 4.14 (q, J=7.3 Hz, 2H), 3.55-3.67 (m, 1H), 3.47-3.52 (m, 2H), 3.37-3.40 (m, 2H), 2.27-2.35 (m, 2H), 1.85-1.92 (m, 2H), 1.70-1.76 (m, 2H), 1.52-1.59 (m, 2H), 1.40 (t, J=7.3 Hz, 3H). m/z 425 (MH⁺).

### Example 53

### Synthesis of 2-fluoro-N-(1-methyl-1H-tetrazol-5-yl)-2-[3-(trifluoromethoxy)phenyl]-7-azaspiro[3.5]nonane-7-carboxamide

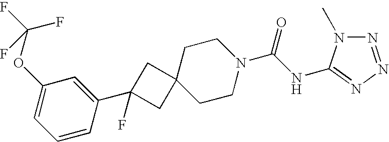

2-Fluoro-2-[3-(trifluoromethoxy)phenyl]-7-azaspiro[3.5]nonane hydrochloride (250 mgs, 0.824 mmol) was dissolved in dioxane (1 mL), and satd sodium bicarbonate (1 mL) and stirred for 10 minutes at r.t. A solution of the nitrophenyl chloroformate (174 mg, 0.865 mmol) in dioxane (1 mL) was added dropwise slowly and the reaction was stirred at room temp for 2 h. The reaction was then diluted with water (2 mL) and the aqueous phase extracted with ethyl acetate (2×5 mL), the organics dried with magnesium sulfate and concentrated. In a separate vial 1-methyl-1H-tetrazol-5-amine (161 mg, 1.62 mmol) was dissolved in DMA (2 mL) at r.t. and sodium hydride (68.1 mg, 1.70 mmol) added. The solution was stirred for 15 minutes at which time a solution of the crude nitrophenyl product (380 mg, 0.811 mmol) in DMA (2 mL) was added dropwise. The reaction was stirred for 1 hour and then quenched with sat. sodium bicarbonate. The reaction was extracted with ethyl acetate (3×10 mL), dried with magnesium sulfate and concentrated. The crude product was purified by flash chromatography to produce the title compound as a light yellow oil. (120 mg, 0.280 mmol, 34%). ¹H NMR (400 MHz, DMSO-d₆) δ ppm 9.94 (1H, br. s.), 7.54-7.60 (1H, m), 7.48-7.54 (1H, m), 7.34-7.42 (2H, m), 3.78 (3H, s), 3.44-3.52 (2H, m), 3.35-3.41 (2H, m), 1.74-1.82 (2H, m), 1.51-1.59 (2H, m). m/z 429.15 (MH⁺).

### Synthesis of 4-nitrophenyl 2-(3-{[5-(trifluoromethyl)pyridin-2-yl]oxy}phenyl)-7-azaspiro[3.5]nonane-7-carboxylate

tert-Butyl 2-(3-{[5-(trifluoromethyl)pyridin-2-yl]oxy}phenyl)-7-azaspiro[3.5]nonane-7-carboxylate (300 mg, 0.649 mmol) was dissolved in dichloromethane (5 mL) and treated with TFA (2 mL). After stirring at room temperature for 90 min, the solvent was evaporated to give 2-(3-{[5-(trifluoromethyl)pyridin-2-yl]oxy}phenyl)-7-azaspiro[3.5]nonane trifluoroacetate (380 mg, 0.64 mmol) which was redissolved in dioxane (10 mL). To this solution was added saturated aqueous sodium bicarbonate (5 mL). After stirring for 5 minutes a solution of para-nitrophenyl chloroformate was added dropwise as a solution in dioxane (3 mL). The bright yellow suspension was stirred at room temperature for 2 h. The mixture was diluted with ethyl acetate and washed with ½ saturated aqueous sodium bicarbonate. The aqueous layer was back extracted once with ethyl acetate. The combined organic layers were washed with brine, then dried (MgSO₄), filtered and concentrated. The residue was purified on silica gel eluting with 20% ethyl acetate/heptane to give the title compound as a pale yellow oil (250 mg, 73%). ¹H NMR (400 MHz, DMSO-d₆) δ ppm 8.46 (d, J=1.7 Hz, 1H), 8.26 (d, J=8.9 Hz, 2H), 7.91 (dd, J=8.7, 2.6 Hz, 1H), 7.39 (t, J=7.7 Hz, 1H), 7.28-7.33 (m, 2H), 7.13 (d, J=7.5 Hz, 1H), 6.95-7.06 (m, 3H), 3.42-3.73 (m, 5H), 2.38 (t, J=10.1 Hz, 2H), 2.00 (t, J=10.6 Hz, 2H), 1.80-1.89 (m, 2H), 1.61-1.70 (m, 2H) m/z 527.7 (MH⁺).

### Example 54

### Synthesis of N-(1-methyl-1H-tetrazol-5-yl)-2-(3-{[5-(trifluoromethyl)pyridin-2-yl]oxy}phenyl)-7-azaspiro[3.5]nonane-7-carboxamide

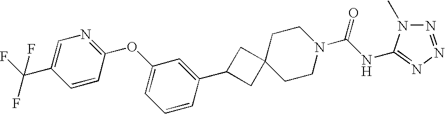

1-Methyl-1H-tetrazol-5-amine (47 mg, 0.47 mmol) was dissolved in anhydrous DMA (2 mL). To this solution was added 60% NaH (20 mg, 0.47 mmol). After stirring at room temperature for 20 min this solution was added to a solution of 4-nitrophenyl 2-(3-{[5-(trifluoromethyl)pyridin-2-yl]oxy}phenyl)-7-azaspiro[3.5]nonane-7-carboxylate in DMA (4 mL). After stirring at room temperature for 4 days under nitrogen additional sodium 1-methyl-1H-tetrazole-5-amine (0.47 mmol) in DMA (2 mL) was added. The mixture was stirred for 5 hours then diluted with ethyl acetate and washed with aqueous saturated sodium bicarbonate. The aqueous layer was back extracted with ethyl acetate and the combined organic layers were washed with brine, dried (MgSO₄), filtered and concentrated. The residue was purified on silica gel eluting with 80% ethyl acetate/heptane increasing polarity to 5% methanol/ethyl acetate. The material was purified a second time on silica gel eluting with 5% methanol/dichloromethane to give the title compound as an orange semi-solid (45 mg, 19%). ¹H NMR (400 MHz, CDCl₃) δ ppm 10.59 (s, 1H), 8.46 (d, J=1.7 Hz, 1H), 7.91 (dd, J=8.5, 2.7 Hz, 1H), 7.38 (t, J=7.9 Hz, 1H), 7.12 (d, J=8.2 Hz, 1H), 6.91-7.07 (m, 3H), 3.97 (s, 3H), 3.66-3.78 (m, 2H), 3.52-3.64 (m, 3H), 2.29-2.41 (m, 2H), 1.91-2.03 (m, 2H), 1.81-1.90 (m, 2H), 1.62-1.71 (m, 2H). m/z 487.8 (MH⁺).

### Example 55

### Synthesis of 2-(2,2-difluoro-1,3-benzodioxol-4-yl)-N-(1-methyl-1H-tetrazol-5-yl)-7-azaspiro[3.5]nonane-7-carboxamide

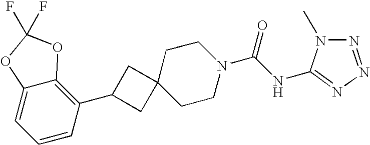

The title compound was prepared from 2-(2,2-difluoro-1,3-benzodioxol-4-yl)-7-azaspiro[3.5]nonane hydrochloride and 1-methyl-1H-tetrazol-5-amine as described for Example 53. The crude residue was dissolved in 1 mL DMSO and purified by reverse phase HPLC (acetonitrile/water/0.1% formic acid) to give the title compound (4 mg). ¹H NMR (400 MHz, DMSO-d₆) δ ppm 7.10-7.27 (m, 3H), 3.76 (s, 3H), 3.63-3.73 (m, 1H), 3.48-3.54 (m, 2H), 3.36-3.41 (m, 2H), 2.31 (t, J=10.3 Hz, 2H), 1.94-2.04 (m, 2H), 1.70-1.77 (m, 2H), 1.51-1.58 (m, 2H). m/z 407 (MH⁺).

### Example 56

### Synthesis of 2-(2,3-difluorophenyl)-N-(1-methyl-1H-tetrazol-5-yl)-7-azaspiro[3.5]nonane-7-carboxamide

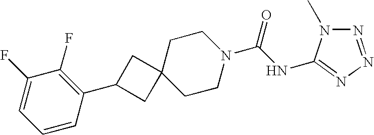

The title compound was prepared from 2-(2,3-difluorophenyl)-7-azaspiro[3.5]nonane hydrochloride and 1-methyl-1H-tetrazol-5-amine as described for Example 53. The crude residue was dissolved in 1 mL DMSO and purified by reverse phase HPLC (acetonitrile/water/0.1% formic acid) to give the title compound (6.7 mg). ¹H NMR (400 MHz, DMSO-d₆) δ ppm 7.11-7.31 (m, 3H), 3.77 (s, 3H), 3.68-3.76 (m, 1H), 3.47-3.54 (m, 2H), 3.37-3.44 (m, 2H), 2.27-2.35 (m, 2H), 1.90-1.99 (m, 2H), 1.70-1.78 (m, 2H), 1.50-1.56 (m, 2H). m/z 363 (MH⁺).

The biological activities of compounds described in the above examples were determined using the following assays.

FAAH Assay

The FAAH assay was carried out in 384-well clear polystyrene plates (Evergreen Scientific) in a total volume of 50 μl per well in a manner similar to that described by Mileni et al., Proc. Nat. Acad. Sci. 2008, 105, 12820-12824. All percents are by volume. Serial dilutions of compound were initially prepared in 100% DMSO, and then diluted two-fold into HPLC-grade H₂O to give 50% DMSO. To each well, was placed the reaction mixture (40 μl) containing 1-4 nM FAAH, 50 mM NaPᵢ, pH 7.4, 3 mM α-ketoglutarate, 0.15 mM NADH, 7.5 U/ml glutamate dehydrogenase, 2 mM ADP, 1 mM EDTA, and 0.1% Triton X-100 (The concentration shown for each component is the final concentration in the assay). To this mixture, was added 5 μl of a compound of Examples 1 to 56 at various concentrations in 50% DMSO (or 5 μl 50% DMSO for controls). This was immediately followed by the addition of 5 μl oleamide (500 μM) dissolved in 75% EtOH/25% DMSO and the reaction mixture was mixed for 1.5 min. The final concentrations of DMSO and EtOH in the assay were each 7.5%. The reactions were incubated at 30° C. and the absorbance at 340 nm was collected over a period of 90 min with readings taken in 30-second intervals using SpectraMax Plus³⁸⁴ Microplate Spectrophotometer (Molecular Devices, Sunnyvale, Calif.). The human FAAH and rat FAAH used in the assay was prepared as described in the patent application WO 2006/067613 using wild-type E. coli cells transformed in St. Louis, Mo., U.S.A. The purity of the enzyme was greater than 98% based on an analysis by SDS-polyacrylamide gel electrophoresis followed by Coomassie Blue staining.

Kinetic Data Analyses

Reaction progress curves were corrected for the non-enzymatic oxidation of NADH by subtraction of absorbancies at each time point obtained from control reactions containing no FAAH enzyme. The loss of enzyme activity as a function of time is well-described by the following mathematical equation (1) for a mono-exponential decay:

Aₜ=A₀+C*e^{(-kobs*1)} (1)

where Aₜ represents the absorbance at time t, A₀ represents the absorbance at time zero, and C represents a constant. Observed rates of enzyme inactivation (k_{obs}) were determined from the non-linear reaction progress curves by fitting the corrected absorbancies to an equation for a mono-exponential decay using the third-party Microsoft Excel plug-in, XLfit (IDBS Limited). Secondary plots of k_{obs} vs. inhibitor concentration were prepared from k_{obs} values obtained from progress curves. Second-order rates for enzyme inactivation, expressed as k_{inact}/Kᵢ (M⁻¹s⁻¹), were calculated from the slopes from linear regression analysis of the secondary plot of k_{obs} vs. inhibitor concentration as defined in following equation (2), where [I]<<Kᵢ:

$$Slope = \frac{{k}_{obs}}{[ l ]} = \frac{{k}_{inact}}{{K}_{i} * ( 1 + [ S ] / {K}_{m} )} \qquad ( 2 )$$

The concentration of substrate in the assay was equal to the Kₘ for oleamide of 50 μM. Therefore, reported k_{inact}/Kᵢ values are obtained by multiplying resulting slopes by a factor of two (i.e. slope=k_{inact}/(Kᵢ*2)).

Table 1, below, lists human FAAH (hFAAH) and rat FAAH (rFAAH) enzyme inhibition values for Examples 1-56 as a ratio of k_{inact}/Kᵢ(M⁻¹s⁻¹).

TABLE 1
In vitro hFAAH and rFAAH k_{inact}/Kᵢ (M⁻¹s⁻¹)
Values for Examples 1-56.

| | hFAAH | rFAAH |
|---|---|---|
| | k_{inact}/Kᵢ | k_{inact}/Kᵢ |
| Ex. | (M⁻¹s⁻¹) | (M⁻¹s⁻¹) |
| 1 | 1570 | 647 |
| 2 | 1760 | 9460 |
| 3 | 1030 | 4920 |
| 4 | 2830 | 3860 |
| 5 | 2190 | 741 |
| 6 | 3060 | 3690 |
| 7 | 2870 | 1620 |
| 8 | 2820 | 1920 |
| 9 | 2450 | 1430 |
| 10 | 2040 | 1820 |
| 11 | 2340 | 9990 |
| 12 | 3950 | 2990 |
| 13 | 3940 | 3560 |
| 14 | 4190 | 5820 |
| 15 | 3460 | 1730 |
| 16 | 9660 | 3200 |
| 17 | 2460 | 3940 |
| 18 | 2640 | 1560 |
| 19 | 2350 | 1370 |
| 20 | 4550 | 3470 |
| 21 | 2700 | 1510 |
| 22 | 5670 | 1340 |
| 23 | 2820 | 1710 |
| 24 | 12400 | 5750 |
| 25 | 2860 | 2340 |
| 26 | 4480 | 1700 |
| 27 | 2940 | 1810 |
| 28 | 2220 | 1060 |
| 29 | 3840 | 2470 |
| 30 | 3240 | 2340 |
| 31 | 1100 | 656 |
| 32 | 2830 | 1320 |
| 33 | 1600 | 636 |
| 34 | 1650 | 1660 |
| 35 | 1530 | 2460 |
| 36 | 2030 | 1160 |
| 37 | 1900 | 1980 |
| 38 | 3720 | 756 |
| 39 | 2670 | 4120 |
| 40 | 3050 | 1540 |
| 41 | 354 | 405 |
| 42 | 991 | 1360 |
| 42a | 1550 | 3040 |
| 42b | 37.1 | 49.4 |
| 42c | 2760 | 3530 |
| 42d | 1100 | 860 |
| 43 | 494 | 247 |
| 44 | 945 | 1340 |
| 45 | 13200 | 4570 |
| 46 | 14100 | 3410 |
| 47 | 15400 | 3330 |
| 48 | 16300 | 5940 |
| 49 | 5240 | 552 |
| 50 | 16700 | 3530 |
| 51 | 2520 | 5460 |
| 52 | 6940 | 3420 |
| 53 | 13900 | 1920 |
| 54 | 21700 | 26800 |
| 55 | 10200 | 2040 |
| 56 | 10300 | 1940 |

In Vivo Complete Freund's Adjuvant (CFA) Efficacy Assay

For additional information on the CFA efficacy assay, see Jayamanne et al., Brit. J. Pharmacol. 2006, 147, 281-288. Experiments were performed on adult Male Sprague-Dawley Rats (200 g-250 g). Inflammation was induced in the left hindpaw of the rat by an intra-plantar injection of 150 uL Complete Freund's Adjuvant (CFA) (SIGMA F5881). The CFA injection immediately induces local inflammation, paw swelling, and pain that persists for at least two weeks post-injection. Baseline paw withdrawal threshold (PWT) was measured to determine the percent inhibition of allodynia using a set of Von Frey Hairs on day 4 post injection as illustrated by the Dixon Up and Down Method (W. J. Dixon, Ann. Rev. Pharmacol. Toxicol. 1980, 20:441-462). Animals that exhibit the pain criteria of 9 grams or less were then placed on study. Test compound was administered at a concentration of 3 mg/kg (mpk) orally with the dosing vehicle 5% N,N′-Dimethylacetamide (SIGMA D137510) and 95% (40% 2-hydroxypropyl-beta-cyclodextrin in water) (SIGMA H107). Following Dose administration PWT threshold was evaluated again at four hours postdose. The Sprague-Dawley rats used in this assay were purchased from Harlan, 8520 Allison Pointe Blvd., Indianapolis, Ind., 46250, U.S.A. Sprague-Dawley rats are an outbred breed of albino rats first produced by the Sprague Dawley farms in Madison, Wis., U.S.A.

Data Analysis

Percent Inhibition of Allodynia was determined by the formula:

$$\% Inhibition of Allodynia = \times \frac{\begin{matrix}Δ PWT ( test compound ) - \\ mean Δ PWT ( vehicle )\end{matrix}}{Baseline - mean Δ PWT ( vehicle )}$$

ΔPWT measurements were averaged for each treatment group and statistical comparisons between groups were made using ANOVA and Dunnett's two-tailed test. Test compounds that increased percent inhibition significantly when compared to the vehicle group (p<0.05 ANOVA/Dunnetts) were determined to be efficacious. Table 2, below, lists CFA efficacy for examples assayed.

TABLE 2
in vivo CFA Efficacy for examples tested at 3 mpk, oral dosing.

| | CFA |
|---|---|
| Ex. | Efficacy^{a,b} |
| 1 | (−)^{c} |
| 2 | (+)^{d} |
| 3 | (+)^{d}/(−)^{e} |
| 4 | (+) |
| 5 | (+)^{c} |
| 6 | (−)^{e} |
| 7 | (−)^{e} |
| 8 | (−)^{e} |
| 9 | (+)^{c} |
| 10 | (−)^{c} |
| 12 | (+) |
| 13 | (+) |
| 14 | (+) |
| 15 | (+) |
| 18 | (−) |
| 19 | (+) |
| 20 | (+) |
| 21 | (+) |
| 22 | (−) |
| 23 | (+) |
| 24 | (+) |
| 25 | (+) |
| 26 | (+) |
| 27 | (+) |
| 28 | (+) |
| 29 | (+) |
| 30 | (+) |
| 32 | (+) |
| 33 | (+) |
| 34 | (+) |
| 36 | (+) |
| 45 | (+) |
| 46 | (+) |
| 47 | (+) |
| 48 | (+) |
| 49 | (+) |
| 50 | (+) |
| 53 | (+) |
| 54 | (+) |

^{a}Test compounds that increased percent inhibition of allodynia significantly when compared to the vehicle group (p < 0.05 ANOVA/Dunnetts) were determined to be efficacious; (+) indicates the test compound was determined to be efficacious at 3 mpk, oral dosing; (−) indicates the test compound was determined to not be efficacious at 3 mpk, oral dosing.
^{b}some compounds were tested at different doses as indicated below.
^{c}compound tested at 10 mpk, intraperitoneal dosing.
^{d}compound tested at 25 mpk, intraperitoneal dosing.
^{e}compound tested at 1 mpk, oral dosing.

1. A compound of Formula I: 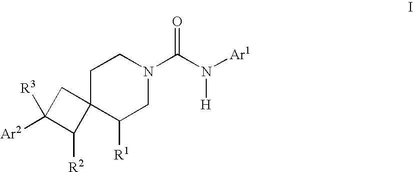
wherein:
Ar¹ is selected from: 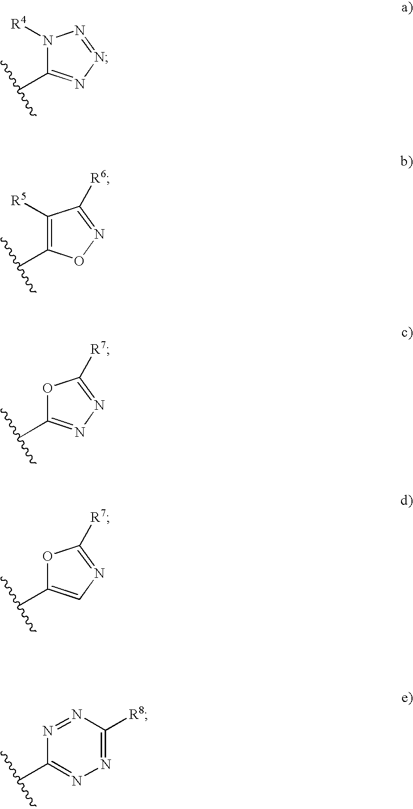
f) benzoisoxazole optionally substituted by 1 to 3 substituents selected from halo, C₁-C₃ alkyl, C₁-C₃ alkoxy, C₁-C₃ haloalkyl or C₁-C₃ haloalkoxy; or
g) pyridine, pyridazine, pyrimidine, or pyrazine; wherein the pyridine, pyridazine, pyrimidine, or pyrazine is optionally substituted by 1 to 3 halo, C₁-C₃ alkyl, —(CH₂)ₙ—(C₃-C₆ cycloalkyl), C₁-C₃ alkoxy, C₁-C₃ haloalkyl or C₁-C₃ haloalkoxy substituents;
Ar² is selected from:
a) phenyl optionally substituted by 1 to 5 substituents selected from halo, C₁-C₆ alkyl, —(CH₂)ₙ—(C₃-C₆ cycloalkyl), C₁-C₆ alkoxy, —(CH₂)ₙ—(C₃-C₆ cycloalkoxy), C₁-C₆ haloalkyl, C₁-C₆ haloalkoxy, —O—CH₂—CH₂—O—(C₁-C₆ alkyl), or —O—CH₂—CH₂—O—(C₁-C₆ haloalkyl); wherein the phenyl is optionally substituted by a substituent of the formulae —R⁹, —O—R⁹, —O—(CH₂)ₚ—R⁹, or —(CH₂)ₚ—O—R⁹;
b) oxazole, isoxazole, thiazole, isothiazole, oxadiazole, or thiadiazole substituted by a substituent of the formulae —(CH₂)ₙ—R⁹, —(CH₂)ₘ—O—R⁹, or —(CH₂)ₚ—O—(CH₂)ₚ—R⁹;
c) a heterocycle of the formula: 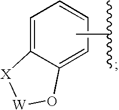
wherein X is CH₂ or O, and W is (CH₂)ₘ or CF₂; or
d) naphthyl, quinolinyl or isoquinolinyl optionally substituted by 1 to 3 halo, C₁-C₃ alkyl, C₁-C₃ alkoxy, C₁-C₃ haloalkyl or C₁-C₃ haloalkoxy substituents;
wherein if Ar¹ is pyridine, pyridazine, pyrimidine, or pyrazine, then Ar² must be phenyl ring substituted by —O—R⁹;
R¹ and R² are independently selected from hydrogen, F, or CH₃;
R³ is hydrogen, CH₃, —O—CH₃, OH, CN, or F;
R⁴ is hydrogen, C₁-C₆ alkyl, —(CH₂)ₙ—(C₃-C₆ cycloalkyl), or C₁-C₆ haloalkyl;
R⁵ is C₁-C₃ alkyl;
R⁶ is hydrogen, C₁-C₆ alkyl, or C₁-C₃ haloalkyl;
R⁷ is C₁-C₃ alkyl, —(CH₂)ₙ—(C₃-C₆ cycloalkyl), R⁹, or —CH₂—O—R⁹;
R⁸ is phenyl optionally substituted by from 1 to 3 substituents selected from halo, C₁-C₃ alkyl, C₁-C₃ alkoxy, C₁-C₃ haloalkyl or C₁-C₃ haloalkoxy groups;
R⁹ is selected from phenyl, naphthyl, or heteroaryl; wherein R⁹ is optionally substituted by from 1 to 3 substituents selected from halo, C₁-C₃ alkyl, —(CH₂)ₙ—(C₃-C₆ cycloalkyl), C₁-C₃ alkoxy, —(CH₂)ₙ—(C₃-C₆ cycloalkoxy), C₁-C₃ haloalkyl, or C₁-C₃ haloalkoxy;
m is 1, 2 or 3; n is 0, 1, 2, 3 or 4; and p is 1 or 2;
or a pharmaceutically acceptable salt thereof.

2. A compound of Formula I according to claim 1 wherein:
Ar¹ is selected from: 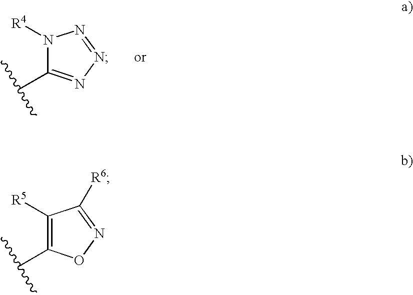
Ar² is selected from:
a) a phenyl ring optionally substituted by from 1 to 3 halo, C₁-C₃ alkyl, —(CH₂)ₙ—(C₃-C₆ cycloalkyl), C₁-C₃ alkoxy, —(CH₂)ₙ—(C₃-C₆ cycloalkoxy), C₁-C₃ haloalkyl, C₁-C₃ haloalkoxy groups; wherein the phenyl ring may also be substituted by a group of the formulae —R⁹, —O—R⁹, —O—CH₂—R⁹, or —O—(CH₂)₂—O—R⁹;
b) a thiazole or oxadiazole ring substituted by a group of the formulae —R⁹; or
c) 2,2-difluoro-1,3-benzodioxole;
R¹ and R² are independently selected from hydrogen, or CH₃;
R³ is hydrogen or F;
R⁴ is C₁-C₃ alkyl;
R⁵ is methyl;
R⁶ is hydrogen or C₁-C₃ alkyl;
R⁹ is phenyl, pyridine, or pyrimidine; wherein the R⁹ ring is optionally substituted by from 1 to 3 groups selected from halo, C₁-C₃ alkyl, —(CH₂)ₙ—(C₃-C₆ cycloalkyl), C₁-C₃ alkoxy, —(CH₂)ₙ—(C₃-C₆ cycloalkoxy), C₁-C₃ haloalkyl or C₁-C₃ haloalkoxy groups;
or a pharmaceutically acceptable salt thereof.

3. A compound of Formula I according to claim 2 wherein:
Ar² is selected from:
a) a phenyl ring optionally substituted by from 1 to 3 groups selected from F, Cl, methyl, ethyl, CF₃, OCH₃, or OCF₃; wherein the phenyl ring may also be substituted by a group of the formulae —O—R⁹ or —O—CH₂—CH₂—O—R⁹;
b) a thiazole or oxadiazole ring substituted by a group of the formulae —R⁹;
R¹ and R² are hydrogen;
R⁴, R⁵, and R⁶ are methyl;
wherein if Ar² is phenyl, R⁹ is pyridine or pyrimidine, the pyridine or pyrimidine being optionally substituted by from 1 to 3 substituents selected from F, Cl, Br, CF₃, or OCF₃; and if Ar² is thiazole or oxadiazole, R⁹ is phenyl optionally substituted by from 1 to 3 substituents selected from F, Cl, Br, CF₃, or OCF₃;
or a pharmaceutically acceptable salt thereof.

4. A compound of claim 1 selected from the group of:
N-pyridazin-3-yl-2-(3-{[5-(trifluoromethyl)pyridin-2-yl]oxy}phenyl)-7-azaspiro[3.5]nonane-7-carboxamide;
N-(3,4-dimethylisoxazol-5-yl)-2-(3-{[5-(trifluoromethyl)pyridin-2-yl]oxy}phenyl)-7-azaspiro[3.5]nonane-7-carboxamide;
N-1,2-benzisoxazol-3-yl-2-(3-{[5-(trifluoromethyl)pyridin-2-yl]oxy}phenyl)-7-azaspiro[3.5]nonane-7-carboxamide;
N-(3,4-dimethylisoxazol-5-yl)-2-[3-(trifluoromethoxy)phenyl]-7-azaspiro[3.5]nonane-7-carboxamide;
N-(3,4-dimethylisoxazol-5-yl)-2-(3-methoxyphenyl)-7-azaspiro[3.5]nonane-7-carboxamide;
N-(3,4-dimethylisoxazol-5-yl)-2-[3-(trifluoromethyl)phenyl]-7-azaspiro[3.5]nonane-7-carboxamide;
N-(3,4-dimethylisoxazol-5-yl)-2-(3-methylphenyl)-7-azaspiro[3.5]nonane-7-carboxamide;
N-(3,4-dimethylisoxazol-5-yl)-2-(3,4-dimethylphenyl)-7-azaspiro[3.5]nonane-7-carboxamide;
2-{3-[(5-bromopyrimidin-2-yl)oxy]phenyl}-N-pyridazin-3-yl-7-azaspiro[3.5]nonane-7-carboxamide;
2-{3-[(5-bromopyridin-2-yl)oxy]phenyl}-N-pyridazin-3-yl-7-azaspiro[3.5]nonane-7-carboxamide;
2-{3-[(5-bromopyridin-2-yl)oxy]phenyl}-N-(3,4-dimethylisoxazol-5-yl)-7-azaspiro[3.5]nonane-7-carboxamide;
N-(3,4-dimethylisoxazol-5-yl)-2-(4-fluoro-3-methylphenyl)-7-azaspiro[3.5]nonane-7-carboxamide;
2-(3-chlorophenyl)-N-(3,4-dimethylisoxazol-5-yl)-7-azaspiro[3.5]nonane-7-carboxamide;
2-(3-chloro-4-fluorophenyl)-N-(3,4-dimethylisoxazol-5-yl)-7-azaspiro[3.5]nonane-7-carboxamide;
N-(3,4-dimethylisoxazol-5-yl)-2-{5-[4-(trifluoromethoxy)phenyl]-1,2,4-oxadiazol-3-yl}-7-azaspiro[3.5]nonane-7-carboxamide;
2-(3-methylphenyl)-N-(1-methyl-1H-tetrazol-5-yl)-7-azaspiro[3.5]nonane-7-carboxamide;
2-(3-methylphenyl)-N-(6-phenyl-1,2,4,5-tetrazin-3-yl)-7-azaspiro[3.5]nonane-7-carboxamide;
N-(3,4-dimethylisoxazol-5-yl)-2-(3-fluoro-5-methylphenyl)-7-azaspiro[3.5]nonane-7-carboxamide;
2-(2,3-difluorophenyl)-N-(3,4-dimethylisoxazol-5-yl)-7-azaspiro[3.5]nonane-7-carboxamide;
2-(3,4-dichlorophenyl)-N-(3,4-dimethylisoxazol-5-yl)-7-azaspiro[3.5]nonane-7-carboxamide;
2-(5-chloro-2-fluorophenyl)-N-(3,4-dimethylisoxazol-5-yl)-7-azaspiro[3.5]nonane-7-carboxamide;
N-(3,4-dimethylisoxazol-5-yl)-2-(3-ethylphenyl)-7-azaspiro[3.5]nonane-7-carboxamide;
N-(3,4-dimethylisoxazol-5-yl)-2-fluoro-2-[3-(trifluoromethoxy)phenyl]-7-azaspiro[3.5]nonane-7-carboxamide;
N-(1-methyl-1H-tetrazol-5-yl)-2-[3-(trifluoromethyl)phenyl]-7-azaspiro[3.5]nonane-7-carboxamide;
2-{3-[2-(4-chlorophenoxy)ethoxy]phenyl}-N-(3,4-dimethylisoxazol-5-yl)-7-azaspiro[3.5]nonane-7-carboxamide;
N-(3,4-dimethylisoxazol-5-yl)-2-[3-(2-phenoxyethoxy)phenyl]-7-azaspiro[3.5]nonane-7-carboxamide;
2-{3-[2-(2-chlorophenoxy)ethoxy]phenyl}-N-(3,4-dimethylisoxazol-5-yl)-7-azaspiro[3.5]nonane-7-carboxamide;
2-(2,2-difluoro-1,3-benzodioxol-4-yl)-N-(3,4-dimethylisoxazol-5-yl)-7-azaspiro[3.5]nonane-7-carboxamide;
2-(3-chloro-2-fluorophenyl)-N-(3,4-dimethylisoxazol-5-yl)-7-azaspiro[3.5]nonane-7-carboxamide;
N-(3,4-dimethylisoxazol-5-yl)-2-[2-fluoro-3-(trifluoromethyl)phenyl]-7-azaspiro[3.5]nonane-7-carboxamide;
N-(3,4-dimethylisoxazol-5-yl)-2-(3′-fluorobiphenyl-3-yl)-7-azaspiro[3.5]nonane-7-carboxamide;
2-(3-chlorophenyl)-N-(3,4-dimethylisoxazol-5-yl)-2-fluoro-7-azaspiro[3.5]nonane-7-carboxamide;
N-(3,4-dimethylisoxazol-5-yl)-2-{5-[4-(trifluoromethyl)phenyl]-1,2,4-oxadiazol-3-yl}-7-azaspiro[3.5]nonane-7-carboxamide;
N-(3-ethyl-4-methylisoxazol-5-yl)-2-[3-(trifluoromethoxy)phenyl]-7-azaspiro[3.5]nonane-7-carboxamide;
N-(5-methyl-1,3,4-oxadiazol-2-yl)-2-[3-(trifluoromethoxy)phenyl]-7-azaspiro[3.5]nonane-7-carboxamide;
N-(3,4-dimethylisoxazol-5-yl)-2-{2-[3-(trifluoromethyl)phenyl]-1,3-thiazol-4-yl}-7-azaspiro[3.5]nonane-7-carboxamide;
N-(3,4-dimethylisoxazol-5-yl)-2-[2-(3-fluorophenyl)-1,3-thiazol-4-yl]-7-azaspiro[3.5]nonane-7-carboxamide;
N-(3,4-dimethylisoxazol-5-yl)-2-{4-[4-(trifluoromethoxy)phenyl]-1,3-thiazol-2-yl}-7-azaspiro[3.5]nonane-7-carboxamide;
2-(4-chloro-3-fluorophenyl)-N-(3,4-dimethylisoxazol-5-yl)-7-azaspiro[3.5]nonane-7-carboxamide;
N-(3,4-dimethylisoxazol-5-yl)-2-{4-[(4-fluorobenzyl)oxy]phenyl}-7-azaspiro[3.5]nonane-7-carboxamide;
2-(3-chloro-4-fluorophenyl)-N-(3,4-dimethylisoxazol-5-yl)-1-methyl-7-azaspiro[3.5]nonane-7-carboxamide;
2-(3-chloro-4-fluorophenyl)-N-(3,4-dimethylisoxazol-5-yl)-5-methyl-7-azaspiro[3.5]nonane-7-carboxamide;
2-(3-chloro-4-fluorophenyl)-N-(3,4-dimethylisoxazol-5-yl)-2-methoxy-7-azaspiro[3.5]nonane-7-carboxamide;
2-(3-chloro-4-fluorophenyl)-N-(3,4-dimethylisoxazol-5-yl)-2-hydroxy-7-azaspiro[3.5]nonane-7-carboxamide;
N-(1-methyl-1H-tetrazol-5-yl)-2-[3-(trifluoromethoxy)phenyl]-7-azaspiro[3.5]nonane-7-carboxamide;
2-(3-chloro-2-fluorophenyl)-N-(1-methyl-1H-tetrazol-5-yl)-7-azaspiro[3.5]nonane-7-carboxamide;
2-[2-fluoro-3-(trifluoromethyl)phenyl]-N-(1-methyl-1H-tetrazol-5-yl)-7-azaspiro[3.5]nonane-7-carboxamide;
2-(3-chloro-4-fluorophenyl)-N-(1-methyl-1H-tetrazol-5-yl)-7-azaspiro[3.5]nonane-7-carboxamide;
N-(1-methyl-1H-tetrazol-5-yl)-2-{5-[4-(trifluoromethyl)phenyl]-1,2,4-oxadiazol-3-yl}-7-azaspiro[3.5]nonane-7-carboxamide;
N-(1-methyl-1H-tetrazol-5-yl)-2-{5-[4-(trifluoromethoxy)phenyl]-1,2,4-oxadiazol-3-yl}-7-azaspiro[3.5]nonane-7-carboxamide;
N-[5-(3-chlorophenyl)-1,3,4-oxadiazol-2-yl]-2-[3-(trifluoromethoxy)phenyl]-7-azaspiro[3.5]nonane-7-carboxamide;
N-(1-ethyl-1H-tetrazol-5-yl)-2-[3-(trifluoromethoxy)phenyl]-7-azaspiro[3.5]nonane-7-carboxamide;
2-fluoro-N-(1-methyl-1H-tetrazol-5-yl)-2-[3-(trifluoromethoxy)phenyl]-7-azaspiro[3.5]nonane-7-carboxamide;
N-(1-methyl-1H-tetrazol-5-yl)-2-(3-{[5-(trifluoromethyl)pyridin-2-yl]oxy}phenyl)-7-azaspiro[3.5]nonane-7-carboxamide;
2-(2,2-difluoro-1,3-benzodioxol-4-yl)-N-(1-methyl-1H-tetrazol-5-yl)-7-azaspiro[3.5]nonane-7-carboxamide; or
2-(2,3-difluorophenyl)-N-(1-methyl-1H-tetrazol-5-yl)-7-azaspiro[3.5]nonane-7-carboxamide;
or a pharmaceutically acceptable salt thereof.

5. A compound of claim 1 selected from the group of:
N-(3,4-dimethylisoxazol-5-yl)-2-[3-(trifluoromethoxy)phenyl]-7-azaspiro[3.5]nonane-7-carboxamide;
2-(3-chloro-4-fluorophenyl)-N-(3,4-dimethylisoxazol-5-yl)-7-azaspiro[3.5]nonane-7-carboxamide;
N-(3,4-dimethylisoxazol-5-yl)-2-{5-[4-(trifluoromethoxy)phenyl]-1,2,4-oxadiazol-3-yl}-7-azaspiro[3.5]nonane-7-carboxamide;
N-(1-methyl-1H-tetrazol-5-yl)-2-[3-(trifluoromethyl)phenyl]-7-azaspiro[3.5]nonane-7-carboxamide;
N-(3,4-dimethylisoxazol-5-yl)-2-[2-fluoro-3-(trifluoromethyl)phenyl]-7-azaspiro[3.5]nonane-7-carboxamide;
N-(3,4-dimethylisoxazol-5-yl)-2-{5-[4-(trifluoromethyl)phenyl]-1,2,4-oxadiazol-3-yl}-7-azaspiro[3.5]nonane-7-carboxamide;
N-(1-methyl-1H-tetrazol-5-yl)-2-[3-(trifluoromethoxy)phenyl]-7-azaspiro[3.5]nonane-7-carboxamide;
2-(3-chloro-2-fluorophenyl)-N-(1-methyl-1H-tetrazol-5-yl)-7-azaspiro[3.5]nonane-7-carboxamide;
2-[2-fluoro-3-(trifluoromethyl)phenyl]-N-(1-methyl-1H-tetrazol-5-yl)-7-azaspiro[3.5]nonane-7-carboxamide;
2-(3-chloro-4-fluorophenyl)-N-(1-methyl-1H-tetrazol-5-yl)-7-azaspiro[3.5]nonane-7-carboxamide;
2-fluoro-N-(1-methyl-1H-tetrazol-5-yl)-2-[3-(trifluoromethoxy)phenyl]-7-azaspiro[3.5]nonane-7-carboxamide;
N-(1-methyl-1H-tetrazol-5-yl)-2-(3-{[5-(trifluoromethyl)pyridin-2-yl]oxy}phenyl)-7-azaspiro[3.5]nonane-7-carboxamide;
2-(2,2-difluoro-1,3-benzodioxol-4-yl)-N-(1-methyl-1H-tetrazol-5-yl)-7-azaspiro[3.5]nonane-7-carboxamide; or
2-(2,3-difluorophenyl)-N-(1-methyl-1H-tetrazol-5-yl)-7-azaspiro[3.5]nonane-7-carboxamide;
or a pharmaceutically acceptable salt thereof.

6. A method of treating pain in a subject, the method comprising administering to a subject in need thereof a therapeutically effective amount of a compound of any one of claims claim 1 to 5, or a pharmaceutically acceptable salt thereof.

7. A method of treating rheumatoid arthritis in a subject, the method comprising administering to a subject in need thereof a therapeutically effective amount of a compound of any one of claims 1 to 5, or a pharmaceutically acceptable salt thereof.

8. A pharmaceutical composition comprising a therapeutically effective amount of a compound of any one of claims 1 to 5, or a pharmaceutically acceptable salt thereof, and a pharmaceutically acceptable carrier.

9. The use of a compound or salt according to any one of claims 1 to 5 in the manufacture of a medicament for the treatment of a condition wherein treatment with a FAAH inhibitor is indicated.

10. A compound or salt according to any one claims 1 to 5 for use in the treatment of a condition wherein treatment with a FAAH inhibitor is indicated.

11. The use according to claim 9 wherein the condition is selected from acute pain, chronic pain, neuropathic pain, nociceptive pain, inflammatory pain, cancer and cancer pain, fibromyalgia, rheumatoid arthritis, inflammatory bowel disease, lupus, diabetes, allergic asthma, vascular inflammation, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, and cardiovascular disease.

12. A compound or salt according to claim 10 wherein the condition is selected from acute pain, chronic pain, neuropathic pain, nociceptive pain, inflammatory pain, cancer and cancer pain, fibromyalgia, rheumatoid arthritis, inflammatory bowel disease, lupus, diabetes, allergic asthma, vascular inflammation, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, and cardiovascular disease.

13. A pharmaceutical composition according to claim 8 for the treatment of a condition wherein treatment with a FAAH inhibitor is indicated.

14. A pharmaceutical composition according to claim 13 wherein the condition is selected from acute pain, chronic pain, neuropathic pain, nociceptive pain, inflammatory pain, cancer and cancer pain, fibromyalgia, rheumatoid arthritis, inflammatory bowel disease, lupus, diabetes, allergic asthma, vascular inflammation, urinary incontinence, overactive bladder, emesis, cognitive disorders, anxiety, depression, sleeping disorders, eating disorders, movement disorders, glaucoma, psoriasis, multiple sclerosis, cerebrovascular disorders, brain injury, gastrointestinal disorders, hypertension, and cardiovascular disease.
